(19) United States
(12) Patent Application Publication
(10) Pub. No.: US 2024/0270843 A1
(43) Pub. Date: Aug. 15, 2024

(54) TREM2 AGONIST BIOMARKERS AND METHODS OF USE THEREOF

(71) Applicant: Vigil Neuroscience, Inc., Watertown, MA (US)

(72) Inventors: Kelley C. LARSON, Watertown, MA (US); Berkley A. LYNCH, Watertown, MA (US); Richard FISHER, Watertown, MA (US); Spyridon PAPAPETROPOULOS, Watertown, MA (US); Evan Andrew THACKABERRY, Watertown, MA (US); Yael MANDELBLAT-CERF, Watertown, MA (US)

(73) Assignee: Vigil Neuroscience, Inc., Watertown, MA (US)

(21) Appl. No.: 18/561,996

(22) PCT Filed: May 27, 2022
(86) PCT No.: PCT/US2022/072605

§ 371 Date: Nov. 17, 2023

Related U.S. Application Data
(60) Provisional application No. 63/202,150, filed on May 28, 2021.
(60) Provisional application No. 63/262,942, filed on Oct. 22, 2021.

(51) Int. Cl. C07K 16/28; A61K 39/00; G01N 33/68

(52) U.S. Cl. CPC C07K 16/2803; G01N 33/6896; A61K 2039/505; G01N 2800/52

(57) ABSTRACT

The present invention provides a method of treating a condition or disorder associated with microglial dysfunction in a human patient, such as Alzheimer's disease, comprising administering to the patient a TREM2 agonist. In another aspect, the invention provides a method of assaying a biological sample taken from a patient having such a condition, such as Alzheimer's disease, for biomarkers characteristic of microglia activity in order to determine treatment benefit or whether the disease has an increased probability of responding to treatment with a TREM2 agonist.

### 1. CROSS-REFERENCE TO RELATED APPLICATIONS

This application claims priority to U.S. Provisional Application No. 63/202,150, filed on May 28, 2021, and 63/262,942, filed on Oct. 22, 2021, the disclosures of which are herein incorporated by reference in their entireties.

### 2. SEQUENCE LISTING

The instant application contains a Sequence Listing which has been submitted electronically in ASCII format and is hereby incorporated by reference in its entirety. Said ASCII copy, created on May 19, 2022, is named 008WO_Sequence_Listing.txt and is 34,106 bytes in size.

### 3. TECHNICAL FIELD OF THE INVENTION

The present invention provides a method of treating a condition or disorder associated with microglial dysfunction in a human patient, such as Alzheimer's disease, comprising administering to the patient a TREM2 agonist. In another aspect, the invention provides a method of assaying a biological sample taken from a patient having such a condition, such as Alzheimer's disease, for biomarkers characteristic of microglia activity in order to determine treatment benefit or whether the disease has an increased probability of responding to treatment with a TREM2 agonist.

### 4. BACKGROUND

Mutations in triggering receptor expressed on myeloid cells 2 (TREM2), a receptor with expression restricted to microglia in the central nervous system, increase the risk of neurodegenerative diseases, such as Alzheimer's disease and frontal temporal dementia (Ulland and Colonna, Nature Reviews, 14, 2018). The TREM2 receptor influences microglial state and function through its interaction with a variety of ligands, and such ligands are critical for sensing tissue damage and minimizing neuropathogenesis (Deczkowska, Cell, 181, 2020). In addition, TREM2 receptor agonism is required for progression of microglia to a neuroprotective, disease-associated (DAM) phenotype (Keren-Shaul Cell, 169, 2017).

There remains a need for methods for treating conditions and disorders related to microglial dysfunction and methods for evaluating and predicting patient responses to such treatments.

### 5. SUMMARY

In one aspect, the present invention provides a method of treating a condition or disorder associated with microglial dysfunction in a human patient, comprising administering to the patient an effective amount of a TREM2 agonist to increase the activity of triggering receptor expressed on myeloid cells 2 (TREM2). In some embodiments, a method of treating Alzheimer's disease is provided.

In another aspect, the present invention provides a method of assaying a biological sample taken from a patient having a condition or disorder associated with microglial dysfunction to determine potential benefit or if the disease has an increased probability of responding to treatment with a TREM2 agonist. Other aspects provide a method of evaluating and monitoring patient biomarker responses to TREM2 agonist therapy. In some embodiments, the TREM2 agonist biomarkers are selected from those shown in Table A. In some embodiments, the TREM2 agonist biomarkers are selected from those shown in Table A*.

### 6. BRIEF DESCRIPTION OF THE DRAWINGS

The drawings show embodiments of the disclosed subject matter for the purpose of illustrating the invention. However, it should be understood that the present application is not limited to the precise arrangements and embodiments shown in the drawings.

FIG. 1 is a set of graphs showing the concentration vs. time profiles for sCSF1R in the CSF of Cynomolgus monkeys after IV administration of antibody Ab-1.

FIG. 2 is a set of graphs showing the concentration vs. time profiles for sCSF1R in the CSF of Cynomolgus monkeys after IT administration of antibody Ab-1.

FIG. 3 is a graph showing the change-from-baseline (CFB) concentration of sCSF1R plotted vs. the concentration of Ab-1 in the CSF of Cynomolgus monkeys after administration of antibody Ab-1.

FIG. 4 is a set of graphs showing the change-from-baseline (CFB) concentration of sCSF1R plotted vs. the concentration of Ab-1 in the CSF of Cynomolgus monkeys after administration of antibody Ab-1, separated out by the post-dose time point when the CSF sample was collected.

FIG. 5 is a set of graphs showing the concentration vs. time profiles for IP-10 in the CSF of Cynomolgus monkeys after IV administration of antibody Ab-1.

FIG. 6 is a set of graphs showing the concentration vs. time profiles for IP-10 in the CSF of Cynomolgus monkeys after IT administration of antibody Ab-1.

FIG. 7 is a set of graphs showing the concentration vs. time profiles for MCP-1 in the CSF of Cynomolgus monkeys after IV administration of antibody Ab-1.

FIG. 8 is a set of graphs showing the concentration vs. time profiles for MCP-1 in the CSF of Cynomolgus monkeys after IT administration of antibody Ab-1.

### 7. DETAILED DESCRIPTION

### 7.1. Definitions

Unless defined otherwise, all technical and scientific terms used herein have the same meaning as commonly understood by one of ordinary skill in the art. Accordingly, the following terms are intended to have the following meanings.

“Agonist” or an “activating” agent, such as a compound or antibody, is an agent that induces (e.g., increases) one or more activities or functions of the target (e.g., TREM2) of the agent after the agent binds the target.

“Antagonist” or a “blocking” agent, such as a compound or antibody, is an agent that reduces or eliminates (e.g., decreases) binding of the target to one or more ligands after the agent binds the target, and/or that reduces or eliminates (e.g., decreases) one or more activities or functions of the target after the agent binds the target. In some embodiments, antagonist agent, or blocking agent, substantially or completely inhibits target binding to one or more of its ligands and/or one or more activities or functions of the target.

“Antibody” is used in the broadest sense and refers to an immunoglobulin or fragment thereof, and encompasses any such polypeptide comprising an antigen-binding fragment or region of an antibody. The recognized immunoglobulin genes include the kappa, lambda, alpha, gamma, delta, epsilon, and mu constant region genes, as well as myriad immunoglobulin variable region genes. Light chains are generally classified as either kappa or lambda. Heavy chains are classified as gamma, mu, alpha, delta, or epsilon, which in turn define the immunoglobulin classes, IgG, IgM, IgA, IgD, and IgE, respectively. Immunoglobulin classes may also be further classified into subclasses, including IgG subclasses IgG₁, IgG₂, IgG₃, and IgG₄; and IgA subclasses IgA₁ and IgA₂. The term includes, but is not limited to, polyclonal, monoclonal, monospecific, multispecific (e.g., bispecific antibodies), natural, humanized, human, chimeric, synthetic, recombinant, hybrid, mutated, grafted, antibody fragments (e.g., a portion of a full-length antibody, generally the antigen binding or variable region thereof, e.g., Fab, Fab′, F(ab′)2, and Fv fragments), and in vitro generated antibodies so long as they exhibit the desired biological activity. The term also includes single chain antibodies, e.g., single chain Fv (sFv or scFv) antibodies, in which a variable heavy and a variable light chain are joined together (directly or through a peptide linker) to form a continuous polypeptide.

The antibodies described herein are, in many embodiments, described by way of their respective polypeptide sequences using single letter amino acid notation. Unless indicated otherwise, polypeptide sequences are provided in N→C orientation.

“Isolated” refers to a change from a natural state, that is, changed and/or removed from its original environment. For example, a polynucleotide or polypeptide (e.g., an antibody) is isolated when it is separated from material with which it is naturally associated in the natural environment. Thus, an “isolated antibody” is one which has been separated and/or recovered from a component of its natural environment.

“Purified antibody” refers to an antibody preparation in which the antibody is at least 80% or greater, at least 85% or greater, at least 90% or greater, at least 95% or greater by weight as compared to other contaminants (e.g., other proteins) in the preparation, such as by determination using SDS-polyacrylamide gel electrophoresis (PAGE) or capillary electrophoresis- (CE) SDS under reducing or non-reducing conditions.

“Extracellular domain” and “ectodomain” are used interchangeably when used in reference to a membrane bound protein and refer to the portion of the protein that is exposed on the extracellular side of a lipid membrane of a cell.

“Binds specifically” in the context of any binding agent, e.g., an antibody, refers to a binding agent that binds specifically to an antigen or epitope, such as with a high affinity, and does not significantly bind other unrelated antigens or epitopes.

“Functional” refers to a form of a molecule which possesses either the native biological activity of the naturally existing molecule of its type, or any specific desired activity, for example as judged by its ability to bind to ligand molecules. Examples of “functional” polypeptides include an antibody binding specifically to an antigen through its antigen-binding region.

“Antigen” refers to a substance, such as, without limitation, a particular peptide, protein, nucleic acid, or carbohydrate which can bind to a specific antibody.

“Epitope” or “antigenic determinant” refers to that portion of an antigen capable of being recognized and specifically bound by a particular antibody. When the antigen is a polypeptide, epitopes can be formed from contiguous amino acids and/or noncontiguous amino acids juxtaposed by tertiary folding of a protein. Linear epitope is an epitope formed from contiguous amino acids on the linear sequence of amino acids. A linear epitope may be retained upon protein denaturing. Conformational or structural epitope is an epitope composed of amino acid residues that are not contiguous and thus comprised of separated parts of the linear sequence of amino acids that are brought into proximity to one another by folding of the molecule, such as through secondary, tertiary, and/or quaternary structures. A conformational or structural epitope may be lost upon protein denaturation. In some embodiments, an epitope can comprise at least 3, and more usually, at least 5 or 8-10 amino acids in a unique spatial conformation. Thus, an epitope as used herein encompasses a defined epitope in which an antibody binds only portions of the defined epitope. There are many methods known in the art for mapping and characterizing the location of epitopes on proteins, including solving the crystal structure of an antibody-antigen complex, competition assays, gene fragment expression assays, mutation assays, and synthetic peptide-based assays, as described, for example, in Using Antibodies: A Laboratory Manual, Chapter 11, Harlow and Lane, eds., Cold Spring Harbor Laboratory Press, Cold Spring Harbor, New York (1999).

“Protein,” “polypeptide,” or “peptide” denotes a polymer of at least two amino acids covalently linked by an amide bond, regardless of length or post-translational modification (e.g., glycosylation, phosphorylation, lipidation, myristoylation, ubiquitination, etc.). Included within this definition are D- and L-amino acids, and mixtures of D- and L-amino acids. Unless specified otherwise, the amino acid sequences of a protein, polypeptide, or peptide are displayed herein in the conventional N-terminal to C-terminal orientation.

“Polynucleotide” and “nucleic acid” are used interchangeably herein and refer to two or more nucleosides that are covalently linked together. The polynucleotide may be wholly comprised of ribonucleosides (i.e., an RNA), wholly comprised of 2′ deoxyribonucleotides (i.e., a DNA) or mixtures of ribo- and 2′ deoxyribonucleosides. The nucleosides will typically be linked together by sugar-phosphate linkages (sugar-phosphate backbone), but the polynucleotides may include one or more non-standard linkages. Non-limiting example of such non-standard linkages include phosphoramidates, phosphorothioates, and amides (see, e.g., Eckstein, F., Oligonucleotides and Analogues: A Practical Approach, Oxford University Press (1992)).

“Operably linked” or “operably associated” refers to a situation in which two or more polynucleotide sequences are positioned to permit their ordinary functionality. For example, a promoter is operably linked to a coding sequence if it is capable of controlling the expression of the sequence. Other control sequences, such as enhancers, ribosome binding or entry sites, termination signals, polyadenylation sequences, and signal sequences are also operably linked to permit their proper function in transcription or translation.

“Amino acid position” and “amino acid residue” are used interchangeably to refer to the position of an amino acid in a polypeptide chain. In some embodiments, the amino acid residue can be represented as “XN”, where X represents the amino acid and the N represents its position in the polypeptide chain. Where two or more variations, e.g., polymorphisms, occur at the same amino acid position, the variations can be represented with a “/” separating the variations. A substitution of one amino acid residue with another amino acid residue at a specified residue position can be represented by XNY, where X represents the original amino acid, N represents the position in the polypeptide chain, and Y represents the replacement or substitute amino acid. When the terms are used to describe a polypeptide or peptide portion in reference to a larger polypeptide or protein, the first number referenced describes the position where the polypeptide or peptide begins (i.e., amino end) and the second referenced number describes where the polypeptide or peptide ends (i.e., carboxy end).

“Polyclonal” antibody refers to a composition of different antibody molecules which is capable of binding to or reacting with several different specific antigenic determinants on the same or on different antigens. A polyclonal antibody can also be considered to be a “cocktail of monoclonal antibodies.” The polyclonal antibodies may be of any origin, e.g., chimeric, humanized, or fully human.

“Monoclonal antibody” refers to an antibody obtained from a population of substantially homogeneous antibodies, i.e., the individual antibodies comprising the population are identical except for possible naturally occurring mutations that may be present in minor amounts. Each monoclonal antibody is directed against a single determinant on the antigen. In some embodiments, monoclonal antibodies to be used in accordance with the present disclosure can be made by the hybridoma method described by Kohler et al., 1975, Nature 256:495-7, or by recombinant DNA methods. The monoclonal antibodies can also be isolated, e.g., from phage antibody libraries.

“Chimeric antibody” refers to an antibody made up of components from at least two different sources. A chimeric antibody can comprise a portion of an antibody derived from a first species fused to another molecule, e.g., a portion of an antibody derived from a second species. In some embodiments, a chimeric antibody comprises a portion of an antibody derived from a non-human animal, e.g., mouse or rat, fused to a portion of an antibody derived from a human. In some embodiments, a chimeric antibody comprises all or a portion of a variable region of an antibody derived from a non-human animal fused to a constant region of an antibody derived from a human.

“Humanized antibody” refers to an antibody that comprises a donor antibody binding specificity, e.g., the CDR regions of a donor antibody, such as a mouse monoclonal antibody, grafted onto human framework sequences. A “humanized antibody” typically binds to the same epitope as the donor antibody.

“Fully human antibody” or “human antibody” refers to an antibody that comprises human immunoglobulin protein sequences only. A fully human antibody may contain murine carbohydrate chains if produced in a non-human cell, e.g., a mouse, in a mouse cell, or in a hybridoma derived from a mouse cell.

“Full-length antibody,” “intact antibody,” or “whole antibody” are used interchangeably to refer to an antibody, such as an anti-TREM2 antibody of the present disclosure, in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

“Antibody fragment” or “antigen-binding moiety” refers to a portion of a full-length antibody, generally the antigen binding or variable domain thereof. Examples of antibody fragments include Fab, Fab′, F(ab′)2, and Fv fragments; diabodies; linear antibodies; single-chain antibodies; and multispecific antibodies formed from antibody fragments that bind two or more different antigens. Several examples of antibody fragments containing increased binding stoichiometries or variable valencies (2, 3 or 4) include triabodies, trivalent antibodies, and trimerbodies, tetrabodies, tandAbs®, di-diabodies, and (sc(Fv)2)₂ molecules, and all can be used as binding agents to bind with high affinity and avidity to soluble antigens (see, e.g., Cuesta et al., 2010, Trends Biotech. 28:355-62).

“Single-chain Fv” or “sFv” antibody fragment comprises the VH and VL domains of an antibody, where these domains are present in a single polypeptide chain. Generally, the Fv polypeptide further comprises a polypeptide linker between the VH and VL domains which enables the sFv to form the desired structure for antigen binding. For a review of sFv, see Pluckthun in The Pharmacology of Monoclonal Antibodies, Vol. 113, pp. 269-315, Rosenberg and Moore, eds., Springer-Verlag, New York (1994).

“Diabodies” refers to small antibody fragments with two antigen-binding sites, which comprise a heavy chain variable domain (VH) connected to a light chain variable domain (VL) in the same polypeptide chain (VH-VL). By using a linker that is short to allow pairing between the two domains on the same chain, the domains are forced to pair with the complementary domains of another chain and create two antigen-binding sites.

“Antigen binding domain” or “antigen binding portion” refers to the region or part of the antigen binding molecule that specifically binds to and complementary to part or all of an antigen. In some embodiments, an antigen binding domain may only bind to a particular part of the antigen (e.g., an epitope), particularly where the antigen is large. An antigen binding domain may comprise one or more antibody variable regions, particularly an antibody light chain variable region (VL) and an antibody heavy chain variable region (VH), and particularly the complementarity determining regions (CDRs) on each of the VH and VL chains.

“Variable region” and “variable domain” are used interchangeably to refer to the polypeptide region that confers the binding and specificity characteristics of each particular antibody. The variable region in the heavy chain of an antibody is referred to as “VH” while the variable region in the light chain of an antibody is referred to as “VL”. The major variability in sequence is generally localized in three regions of the variable domain, denoted as “hypervariable regions” or “CDRs” in each of the VL region and VH region, and forms the antigen binding site. The more conserved portions of the variable domains are referred to as the framework region FR.

“Complementarity-determining region” and “CDR” are used interchangeably to refer to non-contiguous antigen binding regions found within the variable region of the heavy and light chain polypeptides of an antibody molecule. In some embodiments, the CDRs are also described as “hypervariable regions” or “HVR”. Generally, naturally occurring antibodies comprise six CDRs, three in the VH (referred to as: CDRH1 or H1; CDRH2 or H2; and CDRH3 or H3) and three in the VL (referred to as: CDRL1 or L1; CDRL2 or L2; and CDRL3 or L3). The CDR domains have been delineated using various approaches, and it is to be understood that CDRs defined by the different approaches are to be encompassed herein. The “Kabat” approach for defining CDRs uses sequence variability and is the most commonly used (Kabat et al., 1991, “Sequences of Proteins of Immunological Interest, 5^{th} Ed.” NIH 1:688-96). “Chothia” uses the location of structural loops (Chothia and Lesk, 1987, J Mol Biol. 196:901-17). CDRs defined by “AbM” are a compromise between the Kabat and Chothia approach, and can be delineated using Oxford Molecular AbM antibody modeling software (see, Martin et al., 1989, Proc. Natl Acad Sci USA. 86:9268; see also, world wide web www.bioinf-org.uk/abs). The “Contact” CDR delineations are based on analysis of known antibody-antigen crystal structures (see, e.g., MacCallum et al., 1996, J. Mol. Biol. 262, 732-45). The CDRs delineated by these methods typically include overlapping or subsets of amino acid residues when compared to each other.

It is to be understood that the exact residue numbers which encompass a particular CDR will vary depending on the sequence and size of the CDR, and those skilled in the art can routinely determine which residues comprise a particular CDR given the amino acid sequence of the variable region of an antibody.

Kabat, supra, also defined a numbering system for variable domain sequences that is applicable to any antibody. The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU or, Kabat numbering system” or “EU index” is generally used when referring to a residue in an immunoglobulin heavy chain constant region (e.g., the EU index reported in Kabat et al., supra). The “EU index as in Kabat” refers to the residue numbering of the human IgG₁ EU antibody. References to residue numbers in the variable domain of antibodies means residue numbering by the Kabat numbering system. References to residue numbers in the constant domain of antibodies means residue numbering by the EU or, Kabat numbering system (see, e.g., United States Patent Publication No. 2010-280227). One of skill in the art can assign this system of “Kabat numbering” to any variable domain sequence.

Accordingly, unless otherwise specified, references to the number of specific amino acid residues in an antibody or antigen binding fragment are according to the Kabat numbering system.

“Framework region” or “FR region” refers to amino acid residues that are part of the variable region but are not part of the CDRs (e.g., using the Kabat, Chothia or AbM definition). The variable region of an antibody generally contains four FR regions: FR1, FR2, FR3 and FR4. Accordingly, the FR regions in a VL region appear in the following sequence: FR_{L}1-CDRL1-FR_{L}2-CDRL2-FR_{L}3-CDRL3-FR_{L}4, while the FR regions in a VH region appear in the following sequence: FR1_{H}-CDRH1-FR_{H}2-CDRH2-FR_{H}3-CDRH3-FR_{H}4.

“Constant region” or “constant domain” refers to a region of an immunoglobulin light chain or heavy chain that is distinct from the variable region. The constant domain of the heavy chain generally comprises at least one of a CH1 domain, a Hinge (e.g., upper, middle, and/or lower hinge region), a CH2 domain, and a CH3 domain. In some embodiments, the antibody can have additional constant domains CH4 and/or CH5. In some embodiments, an antibody described herein comprises a polypeptide containing a CH1 domain; a polypeptide comprising a CH1 domain, at least a portion of a Hinge domain, and a CH2 domain; a polypeptide comprising a CH1 domain and a CH3 domain; a polypeptide comprising a CH1 domain, at least a portion of a Hinge domain, and a CH3 domain, or a polypeptide comprising a CH1 domain, at least a portion of a Hinge domain, a CH2 domain, and a CH3 domain. In some embodiments, the antibody comprises a polypeptide which includes a CH3 domain. The constant domain of a light chain is referred to a CL, and in some embodiments, can be a kappa or lambda constant region. However, it will be understood by one of ordinary skill in the art that these constant domains (e.g., the heavy chain or light chain) may be modified such that they vary in amino acid sequence from the naturally occurring immunoglobulin molecule.

“Fc region” or “Fc portion” refers to the C terminal region of an immunoglobulin heavy chain. The Fc region can be a native-sequence Fc region or a non-naturally occurring variant Fc region. Generally, the Fc region of an immunoglobulin comprises constant domains CH2 and CH3. Although the boundaries of the Fc region can vary, in some embodiments, the human IgG heavy chain Fc region can be defined to extend from an amino acid residue at position C226 or from P230 to the carboxy terminus thereof. In some embodiments, the “CH2 domain” of a human IgG Fc region, also denoted as “Cγ2”, generally extends from about amino acid residue 231 to about amino acid residue 340. In some embodiments, N-linked carbohydrate chains can be interposed between the two CH2 domains of an intact native IgG molecule. In some embodiments, the CH3 domain” of a human IgG Fc region comprises residues C-terminal to the CH2 domain, e.g., from about amino acid residue 341 to about amino acid residue 447 of the Fc region. A “functional Fc region” possesses an “effector function” of a native sequence Fc region. Exemplary Fc “effector functions” include, among others, Clq binding; complement dependent cytotoxicity (CDC); Fc receptor binding; antibody dependent cell-mediated cytotoxicity (ADCC); phagocytosis; down regulation of cell-surface receptors (e.g., LT receptor); etc. Such effector functions generally require the Fc region to be combined with a binding domain (e.g., an antibody variable domain) and can be assessed using various assays known in the art.

“Native sequence Fc region” comprises an amino acid sequence identical to the amino acid sequence of an Fc region found in nature. Native sequence human Fc regions include a native sequence human IgG₁ Fc region (non-A and A allotypes); native sequence human IgG₂ Fc region; native sequence human IgG₃ Fc region; and native sequence human IgG₄ Fc region, as well as naturally occurring variants thereof.

“Variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Affinity-matured” antibody, such as an affinity matured anti-TREM2 antibody of the present disclosure, is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology, 1992, 10:779-783 describes affinity maturation by VH- and VL-domain shuffling. Random mutagenesis of HVR and/or framework residues is described by, for example: Barbas et al., Proc Nat. Acad. Sci. USA., 1994, 91:3809-3813; Schier et al. Gene, 1995, 169: 147-155; Yelton et al., Immunol., 1995, 155: 1994-2004; Jackson et al., Immunol., 1995, 154(7):3310-9; and Hawkins et al, J. Mol. Biol., 1992, 226:889-896.

“Binding affinity” refers to strength of the sum total of noncovalent interactions between a ligand and its binding partner. In some embodiments, binding affinity is the intrinsic affinity reflecting a one-to-one interaction between the ligand and binding partner. The affinity is generally expressed in terms of equilibrium association (K_{A}) or dissociation constant (K_{D}), which are in turn reciprocal ratios of dissociation (k_{off}) and association rate constants (kₒₙ).

“Percent (%) sequence identity” and “percentage sequence homology” are used interchangeably herein to refer to comparisons among polynucleotides or polypeptides, and are determined by comparing two optimally aligned sequences over a comparison window, wherein the portion of the polynucleotide or polypeptide sequence in the comparison window may comprise gaps as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv Appl Math. 2:482, by the homology alignment algorithm of Needleman and Wunsch, 1970, J Mol Biol. 48:443, by the search for similarity method of Pearson and Lipman, 1988, Proc Natl Acad Sci USA. 85:2444-8, and particularly by computerized implementations of these algorithms (e.g., BLAST, ALIGN, GAP, BESTFIT, FASTA, and TFASTA; see, e.g., Mount, D. W., Bioinformatics: Sequence and Genome Analysis, 2^{nd} Ed., Cold Spring Harbor Laboratory Press, Cold Spring Harbor, New York (2013)).

Examples of algorithms that are suitable for determining percent sequence identity and sequence similarity are the BLAST and BLAST 2.0, FASTDB, or ALIGN algorithms, which are publicly available (e.g., NCBI: National Center for Biotechnology Information). Those skilled in the art can determine appropriate parameters for aligning sequences. For example, the BLASTN program (for nucleotide sequences) can use as defaults a word length (W) of 11, an expectation (E) of 10, M=5, N=−4, and a comparison of both strands. Comparison of amino acid sequences using BLASTP can use as defaults a word length (W) of 3, an expectation (E) of 10, and the BLOSUM62 scoring matrix (see Henikoff and Henikoff, 1989, Proc Natl Acad Sci USA. 89:10915-9).

“Amino acid substitution” refers to the replacement of one amino acid in a polypeptide with another amino acid. A “conservative amino acid substitution” refers to the interchangeability of residues having similar side chains, and thus typically involves substitution of the amino acid in the polypeptide with amino acids within the same or similar defined class of amino acids. By way of example and not limitation, an amino acid with an aliphatic side chain may be substituted with another aliphatic amino acid, e.g., alanine, valine, leucine, isoleucine, and methionine; an amino acid with hydroxyl side chain is substituted with another amino acid with a hydroxyl side chain, e.g., serine and threonine; an amino acid having aromatic side chains is substituted with another amino acid having an aromatic side chain, e.g., phenylalanine, tyrosine, tryptophan, and histidine; an amino acid with a basic side chain is substituted with another amino acid with a basic side chain, e.g., lysine, arginine, and histidine; an amino acid with an acidic side chain is substituted with another amino acid with an acidic side chain, e.g., aspartic acid or glutamic acid; and a hydrophobic or hydrophilic amino acid is replaced with another hydrophobic or hydrophilic amino acid, respectively.

“Amino acid insertion” refers to the incorporation of at least one amino acid into a predetermined amino acid sequence. An insertion can be the insertion of one or two amino acid residues; however, larger insertions of about three to about five, or up to about ten or more amino acid residues are contemplated herein.

“Amino acid deletion” refers to the removal of one or more amino acid residues from a predetermined amino acid sequence. A deletion can be the removal of one or two amino acid residues; however, larger deletions of about three to about five, or up to about ten or more amino acid residues are contemplated herein.

“Subject” refers to a mammal, including, but not limited to, humans, non-human primates, and non-primates, such as goats, horses, and cows. In some embodiments, the terms “subject” and “patient” are used interchangeably herein in reference to a human subject.

“Therapeutically effective dose,” “therapeutically effective amount,” or “effective dose” refer to that quantity of a compound, including a biologic compound, or pharmaceutical composition, that is sufficient to result in a desired activity upon administration to a mammal in need thereof. As used herein, with respect to the pharmaceutical compositions comprising an antibody, the term “therapeutically effective amount/dose” refers to the amount/dose of the antibody or pharmaceutical composition thereof that is sufficient to produce an effective response upon administration to a mammal.

“Pharmaceutically acceptable” refers to compounds or compositions which are generally safe, non-toxic and neither biologically nor otherwise undesirable, and includes a compound or composition that is acceptable for human pharmaceutical and veterinary use. The compound or composition may be approved or approvable by a regulatory agency or listed in the U.S. Pharmacopeia or other generally recognized pharmacopeia for use in animals, including humans.

“Pharmaceutically acceptable excipient, carrier or adjuvant” refers to an excipient, carrier or adjuvant that can be administered to a subject, together with at least one therapeutic agent (e.g., an antibody of the present disclosure), and which does not destroy the pharmacological activity thereof and is generally safe, nontoxic and neither biologically nor otherwise undesirable when administered in doses sufficient to deliver a therapeutic amount of the agent.

The term “treatment” is used interchangeably herein with the term “therapeutic method” and refers to both: 1) therapeutic treatments or measures that cure, slow down, lessen symptoms of, and/or halt progression of a diagnosed pathologic condition, disease, or disorder, and 2) prophylactic/preventative measures. Those in need of treatment may include individuals already having a particular medical disease or disorder as well as those who may ultimately acquire the disorder (i.e., those at risk or needing preventive measures).

The term “subject” or “patient” as used herein refers to any individual to which the subject methods are performed. Generally, the subject is human, although as will be appreciated by those in the art, the subject may be any animal.

In some embodiments, compounds of the present invention are able to cross the blood-brain barrier (BBB). The term “blood-brain barrier” or “BBB”, as used herein refers to the BBB proper as well as to the blood-spinal barrier. The blood-brain barrier, which consists of the endothelium of the brain vessels, the basal membrane, and neuroglial cells, acts to limit penetration of substances into the brain. In some embodiments, the brain/plasma ratio of total drug is at least approximately 0.01 after administration (e.g., oral or intravenous administration) to a patient. In some embodiments, the brain/plasma ratio of total drug is at least approximately 0.03. In some embodiments, the brain/plasma ratio of total drug is at least approximately 0.06. In some embodiments, the brain/plasma ratio of total drug is at least approximately 0.1. In some embodiments, the brain/plasma ratio of total drug is at least approximately 0.2.

The term “homologue,” especially “TREM homologue,” as used herein refers to any member of a series of peptides or nucleic acid molecules having a common biological activity, including antigenicity/immunogenicity and inflammation regulatory activity, and/or structural domain and having sufficient amino acid or nucleotide sequence identity as defined herein. TREM homologues can be from either the same or different species of animals.

The term “variant” as used herein refers either to a naturally occurring allelic variation of a given peptide or a recombinantly prepared variation of a given peptide or protein in which one or more amino acid residues have been modified by amino acid substitution, addition, or deletion.

The term “derivative” as used herein refers to a variation of given peptide or protein that are otherwise modified, i.e., by covalent attachment of any type of molecule, preferably having bioactivity, to the peptide or protein, including non-naturally occurring amino acids.

The abbreviation “NFL” as used herein refers to “neurofilament light chain”.

The abbreviation “sCSF1R” as used herein refers to “soluble colony stimulating factor 1 receptor”.

The abbreviation “sTREM2” as used herein refers to “soluble Triggering Receptor Expressed On Myeloid Cells 2”.

The abbreviation “SPP1” as used herein refers to “secreted phosphoprotein 1”, which is a synonym for and is interchangeable with the term osteopontin (OPN).

The abbreviation “IL-IRA” as used herein refers to “Interleukin-1 Receptor Antagonist” and is used interchangeably herein with the term “IL-1RN” which refers to the gene that encodes for protein IL-1RA.

The abbreviation “CSF2” as used herein refers to “colony stimulating factor 2”, which is a synonym for and is interchangeable with the abbreviation “GM-CSF” which refers to “granulocyte-macrophage colony-stimulating factor”.

The abbreviation “IP10” as used herein refers to “Interferon gamma-induced protein 10”, which is a synonym for and is interchangeable with the abbreviation “CXCL10” which refers to “C—X—C motif chemokine ligand 10”.

### Miscellaneous Small Molecule Definitions

This section will define additional terms used to describe the scope of the compounds, compositions and uses disclosed herein.

The terms “C₁₋₃alkyl,” “C₁₋₅alkyl,” and “C₁₋₆alkyl” as used herein refer to a straight or branched chain hydrocarbon containing from 1 to 3, 1 to 5, and 1 to 6 carbon atoms, respectively. Representative examples of C₁₋₃alkyl, C₁₋₅alky, or C₁₋₆alkyl include, but are not limited to, methyl, ethyl, n-propyl, iso-propyl, n-butyl, sec-butyl, iso-butyl, tert-butyl, pentyl and hexyl.

The term “C₂₋₄alkenyl” as used herein refers to a saturated hydrocarbon containing 2 to 4 carbon atoms having at least one carbon-carbon double bond. Alkenyl groups include both straight and branched moieties. Representative examples of C₂₋₄alkenyl include, but are not limited to, 1-propenyl, 2-propenyl, 2-methyl-2-propenyl, and butenyl.

The term “C₃₋₆cycloalkyl” as used herein refers to a saturated carbocyclic molecule wherein the cyclic framework has 3 to 6 carbon atoms. Representative examples of C₃₋₅cycloalkyl include, but are not limited to, cyclopropyl, cyclobutyl, cyclopentyl, and cyclohexyl.

The terms “diC₁₋₃alkylamino” as used herein refer to —NR*R**, wherein R* and R** independently represent a C₁₋₃alkyl as defined herein. Representative examples of diC₁₋₃alkylamino include, but are not limited to, —N(CH₃)2, —N(CH₂CH₃)2, —N(CH₃)(CH₂CH₃), —N(CH₂CH₂CH₃)2, and —N(CH(CH₃)2)₂.

The term “C₁₋₃alkoxy” and “C₁₋₆alkoxy” as used herein refer to —OR^{#}, wherein R^{#} represents a C₁₋₃alkyl and C₁₋₆alkyl group, respectively, as defined herein. Representative examples of C₁₋₃alkoxy or C₁₋₆alkoxy include, but are not limited to, methoxy, ethoxy, propoxy, iso-propoxy, and butoxy.

The term “halogen” as used herein refers to —F, —Cl, —Br, or —I.

The term “halo” as used herein as a prefix to another term for a chemical group refers to a modification of the chemical group, wherein one or more hydrogen atoms are substituted with a halogen as defined herein. The halogen is independently selected at each occurrence. For example, the term “C₁₋₆haloalkyl” refers to a C₁₋₆alkyl as defined herein, wherein one or more hydrogen atoms are substituted with a halogen. Representative examples of C₁₋₆haloalkyl include, but are not limited to, —CH₂F, —CHF₂, —CF₃, —CHFCl, —CH₂CF₃, —CFHCF₃, —CF₂CF₃, —CH(CF₃)₂, —CF(CHF₂)₂, and —CH(CH₂F)(CF₃). Further, the term “C₁₋₆haloalkoxy” for example refers to a C₁₋₆alkoxy as defined herein, wherein one or more hydrogen atoms are substituted with a halogen. Representative examples of C₁₋₆haloalkoxy include, but are not limited to, —OCH₂F, —OCHF₂, —OCF₃, —OCHFCl, —OCH₂CF₃, —OCFHCF₃, —OCF₂CF₃, —OCH(CF₃)₂, —OCF(CHF₂)2, and —OCH(CH₂F)(CF₃).

The term “5-membered heteroaryl” or “6-membered heteroaryl” as used herein refers to a 5 or 6-membered carbon ring with two or three double bonds containing one ring heteroatom selected from N, S, and O and optionally one or two further ring N atoms instead of the one or more ring carbon atom(s). Representative examples of a 5-membered heteroaryl include, but are not limited to, furyl, imidazolyl, pyrazolyl, isoxazolyl, isothiazolyl, oxadiazolyl, and oxazolyl. Representative examples of a 6-membered heteroaryl include, but are not limited to, pyridyl, pyrimidyl, pyrazyl, and pyridazyl.

The term “C₃₋₆heterocycloalkyl” as used herein refers to a saturated carbocyclic molecule wherein the cyclic framework has 3 to 6 carbons and wherein one carbon atom is substituted with a heteroatom selected from N, O, and S. If the C₃₋₆heterocycloalkyl group is a C₆heterocycloalkyl, one or two carbon atoms are substituted with a heteroatom independently selected from N, O, and S. Representative examples of C₃₋₆heterocycloalkyl include, but are not limited to, aziridinyl, azetidinyl, oxetanyl, pyrrolidinyl, piperazinyl, morpholinyl, and thiomorpholinyl.

The term “C₅₋₈spiroalkyl” as used herein refers a bicyclic ring system, wherein the two rings are connected through a single common carbon atom. Representative examples of C₅₋₈spiroalkyl include, but are not limited to, spiro[2.2]pentanyl, spiro[3.2]hexanyl, spiro[3.3]heptanyl, spiro[3.4]octanyl, and spiro[2.5]octanyl.

The term “C₅₋₈tricycloalkyl” as used herein refers a tricyclic ring system, wherein all three cycloalkyl rings share the same two ring atoms. Representative examples of C₅₋₈tricycloalkyl include, but are not limited to, tricyclo[1.1.1.0^{1,3}]pentanyl,

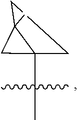

tricyclo[2.1.1.0^{1,4}]hexanyl, tricyclo[3.1.1.0^{1,5}]hexanyl, and tricyclo[3.2.1.0^{1,5}]octanyl.

The term “aryl” used alone or as part of a larger moiety as in “aralkyl,” “aralkoxy,” or “aryloxyalkyl,” refers to monocyclic or bicyclic ring systems having a total of 4 to 14 ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl, and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” in the context of “heteroaryl” particularly includes, but is not limited to, nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic or bicyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As described herein, compounds of the present disclosure may contain “substituted” moieties. In general, the term “substituted” means that one or more hydrogens of the designated moiety are replaced with a suitable substituent. Unless otherwise indicated, an “optionally substituted” group may have a suitable substituent at one or more substitutable position of the group, and when more than one position in any given structure is substituted with more than one substituent selected from a specified group, the substituent may be either the same or different at every position. Combinations of substituents envisioned by the present disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

The term “pharmaceutically acceptable” as used herein refers to generally recognized for use in subjects, particularly in humans.

The term “pharmaceutically acceptable salt” as used herein refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, and the like; or (2) salts formed when an acidic proton present in the parent compound either is replaced by a metal ion, for example, an alkali metal ion, an alkaline earth ion, or an aluminum ion; or coordinates with an organic base such as ethanolamine, diethanolamine, triethanolamine, N-methylglucamine, dicyclohexylamine, and the like. Additional examples of such salts can be found in Berge et al., J. Pharm. Sci. 66(1):1-19 (1977). See also Stahl et al., Pharmaceutical Salts: Properties, Selection, and Use, 2^{nd} Revised Edition (2011).

### 7.2. General Description of Certain Embodiments of the Invention

Diagnosis, prognosis, and treatment of conditions and disorders associated with microglial dysfunction, such as Alzheimer's disease, in a patient is greatly aided by the identification of changes in levels and types of cells in the plaque microenvironment, expression patterns for sets of genes of cells associated with the plaque microenvironment, cytokine expression levels, immunological response factors, or other changes in the plaque microenvironment, referred to herein generally as “biomarkers” or more specifically in relation to gene expression patterns as “gene signatures,” “gene expression biomarkers,” or “molecular signatures,” or in relation to protein expression patterns as “protein signatures,” “protein expression biomarkers,” or “proteome signatures,” or in relation to cell-type composition patterns as “cell signatures” (i.e., microglial cell signatures), which are characteristic of said conditions and disorders. Such biomarkers may be associated with clinical outcomes. If such an association is predictive of a clinical response, the biomarker is advantageously used in methods of selecting or stratifying patients as more (or less, as the case may be) likely to benefit from a treatment regimen, such as one of those disclosed herein.

Biological samples from a patient with biomarker profiles that are predictive of a positive response to treatment are referred to herein as “biomarker positive” or “biomarker high.” Conversely, biological samples from a patient with biomarker profiles that are not predictive of a positive response are referred to herein as “biomarker negative” or “biomarker low.” Alternative terms can be used depending upon the biomarker, but a higher amount, or “biomarker high,” usually can be described using alternative terminology, such as “biomarker positive” or “biomarker +,” while a lower amount of a biomarker or “biomarker low” usually can be described using alternative terminology, such as “biomarker negative” or “biomarker −.” In some embodiments, biomarker results may also be classified as “normal”, wherein the level of the biomarker in the biological sample from a patient is approximately the same as the level of the biomarker in a control biological sample, or as “abnormal”, wherein the level of the biomarker in the biological sample from a patient deviates from the level of the biomarker in a control biological sample to a statistically significant degree after accounting for any number of appropriate factors.

In some embodiments, a biomarker used in the present invention is a biomarker panel, such as a gene expression panel. In other embodiments, a biomarker panel is a cytokine panel. In other embodiments a biomarker panel is a characterization of cell types present in the plaque microenvironment.

Such a “panel,” as used herein refers to a group of specific biomarkers, e.g., specific genes or specific cell type populations in the plaque microenvironment, that respond to a particular stimulus (e.g., treatment of the patient with a TREM2 agonist), in a way that tends to predict the likelihood of a particular clinical outcome. Individual biomarkers, e.g., expression of a gene or prevalence of a particular cell type, in a panel need not each respond in the same way. Some may be up-regulated, some may be down-regulated, and some may remain unchanged; accordingly, the overall response of the panel is generally the most useful in predicting the likelihood of a clinical response.

In some embodiments, a biomarker used in the present invention is a gene signature. In other embodiments, a biomarker is a cytokine signature. In other embodiments a biomarker panel is a cell type signature of cells in the plaque microenvironment. Similar to a panel, a “signature” as used herein refers to a group of biomarkers, such as specific genes or specific cell type populations present in the plaque microenvironment, that respond to a stimulus to provide a fingerprint (distinctive pattern) of biomarker response to treatment.

Furthermore, while biomarkers derived from a patient suffering from a condition or disorder associated with microglial dysfunction are an important tool in improving the diagnosis, prognosis, and treatment of said condition or disorder, the invasiveness of collecting biological samples may increase the risk of serious complications, including anesthetic catastrophes, hemorrhage, infection, seizures, and death (Warren et al, Brain, 2005). Both the surgical removal of brain tissue (biopsy) and the aspiration of cells from plaque sites (fine needle aspiration cytology) have the potential to expose abnormal cells to the cranial cavity. The reduced invasiveness of collecting serum samples for biomarker analysis relative to biopsy allows for more continuous monitoring of patient response to treatment. Consequently, minimally invasive diagnostic tools and methods that avoid disrupting cranial integrity or causing inflammation, such as “serum biomarkers,” present opportunities to improve patient care while mitigating risks associated with current treatment regimens. Serum biomarkers include biomarkers that may be obtained by a bodily fluid sample obtained remote from plaque sites (e.g., venous blood and lymph fluid). Examples of serum biomarkers include, for example, circulating cytokines and growth factors, as well as phenotypic and genotypic markers in circulating immune cells.

In some embodiments, biomarkers may be associated with safety related parameters of treatment. In some embodiments, one or more biomarkers of the present disclosure can be advantageously used in methods of monitoring the safety of a treatment regimen if the biomarkers are predictive of patient safety related outcomes of the treatment. In some embodiments, the one or more biomarkers predictive of the safety of the treatment regimen are independent of biomarkers that are predictive of clinical response. In some embodiments, the one or more biomarkers predictive of the safety of the treatment regimen are the same as the biomarkers predictive of clinical response.

The present invention generally relates to the surprising discovery that the expression levels of certain genes associated with microglial activity are altered after treatment with a TREM2 agonist. It has been found that a TREM2 agonist monoclonal antibody and a small molecule TREM2 agonist were capable of passing the blood brain barrier in a mouse model and were found to activate microglia in a selective manner, while not affecting neurons or astrocytes.

### 7.3. Disease-Related Biomarkers

It has been surprisingly found that administration of a TREM2 agonist to a mouse model expressing human TREM2 yields up-regulation of a number of genes involved in multiple pathways associated with microglial function, homeostasis, and survival. The up-regulation or down-regulation of these genes or proteins can be used as biomarkers in methods described herein, such as methods of treating patients suffering from a condition or disorder associated with microglial dysfunction, diagnosing such a condition or disorder, or predicting patient response to treatment of such a condition or disorder.

Each table below is to be understood to include any single biomarker listed in said table, or any combination of two or more biomarkers listed in said table, in all possible permutations and combinations, or every biomarker listed in said table. All possible combinations of two or more biomarkers listed in each of the tables below are contemplated as part of the disclosure of the present application.

In some embodiments, a biomarker useful in the methods of the present disclosure is one or more of those listed in Table A:

TABLE A
Selected Genes

Adamts16
AI464131
Apex1
Arc
Atg14
Atp6v0e
Axl
Bad
Bard1
Bcl2
Bcl2a1a
Birc5
Blnk
Bnip3
Bnip31
Btk
C3
C3ar1
C4a
C5ar1
Cables1
Camk4
Casp4
Casp6
Casp7
Casp8
Ccr5
Cd14
Cd40
Cd68
Cd72
Cd83
Cd84
Cd86
Cdk20
Cdkn1c
Cflar
Cks1b
Clic4
Clstn1
Cnn2
Csf1
Csflr
Csf2rb
Ctss
Cx3crl
Dab2
Ddx58
Dna2
Dock1
Dock2
Dusp7
E2f1
Eed
Emcn
Ercc2
Ezh2
Fcer1g
Fcgr1
Fcrls
Fen1
Fscn1
Gal3st1
Gbp2
Gna15
Gpr183
Gpr34
Gria2
Grm3
Grn
Gsn
Gstm1
Hdac1
Hdac4
Hells
Homer1
Hpgds
Ifi30
Ifih1
Ifitm2
Ifnar2
Il10rb
Il1a
Il1rl2
Il2rg
Inpp5d
Iqsec1
Irak1
Irf1
Irf3
Irf8
Itgam
Itgav
Itgb5
Jun
Kat2b
Kcnj10
Kdm4d
Kdm5c
Lacc1
Lair1
Lamp1
Lamp2
Lgmn
Lig1
Lilrb4a
Lingo1
Lmnb1
Ltbr
Ly6a
Lyn
Man2b1
Map1lc3a
Map3k1
Mapt
Mavs
Mb21d1
Mbd2
Mcm2
Mcm5
Mcm6
Mdm2
Mertk
Mmp14
Mpeg1
Msh2
Msn
Mvp
Ncaph
Ncf1
Nfkb1
Nfkb2
Ngf
Nlgn2
Nostrin
Nqo1
Osmr
P2rx7
Pacsin1
Pak1
Pcna
Pdpn
Pik3cb
Pik3r2
Pla2g4a
Pla2g5
Plcg2
Plxdc2
Pole
Prdx1
Prkcq
Pros1
Psmb8
Ptpn6
Ptprc
Rab6b
Rac2
Rad1
Rad51
Rapgef3
Rb1cc1
Rela
Rnf8
Rp128
Rpl36al
Rps2
Rps9
Rrm2
Rsad2
S1pr3
Sall1
Serpina3n
Siglecf
Sirt1
Slamf9
Slc6a1
Slco2b1
Slfn8
Smc1a
Sox9
Srgn
Stat1
Stx18
Suz12
Tcirg1
Tgfa
Tgfb1
Tgfbr1
Tgm2
Tie1
Tlr4
Tm4sf1
Tmem173
Tmem206
Tmem37
Tmem64
Tnfrsf12a
Tnfrsf1a
Top2a
Traf1
Traf2
Trim47
Trp73
Tubb3
Txnrd1
Tyrobp
Vav1
Vim

In some embodiments, a biomarker useful in the methods of the present disclosure is one or more of those listed in Table A*:

TABLE A*
Selected Genes and proteins

| | | | | | |
|---|---|---|---|---|---|
| Adamts16 | Cdkn1c | Gsn | Lilrb4a | Pcna | Slc6a1 |
| AI464131 | Cflar | Gstm1 | Lingo1 | Pdpn | Slco2b1 |
| Apex1 | Cks1b | Hdac1 | Lmnb1 | Pik3cb | Slfn8 |
| Arc | Clic4 | Hdac4 | Ltbr | Pik3r2 | Smc1a |
| Atg14 | Clstn1 | Hells | Ly6a | Pla2g4a | Sox9 |
| Atp6v0e | Cnn2 | Homer1 | Lyn | Pla2g5 | Srgn |
| Axl | Csf1 | Hpgds | Man2b1 | Plcg2 | Stat1 |
| Bad | Csf1r | Ifi30 | Map1lc3a | Plxdc2 | Stx18 |
| Bard1 | Csf2rb | Ifih1 | Map3k1 | Pole | Suz12 |
| Bcl2 | Ctss | Ifitm2 | Mapt | Prdx1 | Tcirg1 |
| Bcl2a1a | Cx3cr1 | Ifnar2 | Mavs | Prkcq | Tgfa |
| Birc5 | Dab2 | Il10rb | Mb21d1 | Pros1 | Tgfb1 |
| Blnk | Ddx58 | Il1a | Mbd2 | Psmb8 | Tgfbr1 |
| Bnip3 | Dna2 | Il1rl2 | Mcm2 | Ptpn6 | Tgm2 |
| Bnip31 | Dock1 | Il2rg | Mcm5 | Ptprc | Tie1 |
| Btk | Dock2 | Inpp5d | Mcm6 | Rab6b | Tlr4 |
| C3 | Dusp7 | Iqsec1 | Mdm2 | Rac2 | Tm4sf1 |
| C3ar1 | E2f1 | Irak1 | Mertk | Rad1 | Tmem173 |
| C4a | Eed | Irf1 | Mmp14 | Rad51 | Tmem206 |
| C5ar1 | Emcn | Irf3 | Mpeg1 | Rapgef3 | Tmem37 |
| Cables1 | Ercc2 | Irf8 | Msh2 | Rb1cc1 | Tmem64 |
| Camk4 | Ezh2 | Itgam | Msn | Rela | Tnfrsf12a |
| Casp4 | Fcer1g | Itgav | Mvp | Rnf8 | Tnfrsf1a |
| Casp6 | Fcgr1 | Itgb5 | Ncaph | Rpl28 | Top2a |
| Casp7 | Fcrls | Jun | Ncf1 | Rpl36al | Traf1 |
| Casp8 | Fen1 | Kat2b | Nfkb1 | Rps2 | Traf2 |
| Ccr5 | Fscn1 | Kcnj10 | Nfkb2 | Rps9 | Trim47 |
| Cd14 | Gal3st1 | Kdm4d | Ngf | Rrm2 | Trp73 |
| Cd40 | Gbp2 | Kdm5c | Nlgn2 | Rsad2 | Tubb3 |
| Cd68 | Gna15 | Lacc1 | Nostrin | S1pr3 | Txnrd1 |
| Cd72 | Gpr183 | Lair1 | Nqo1 | Sall1 | Tyrobp |
| Cd83 | Gpr34 | Lamp1 | Osmr | Serpina3n | Vav1 |
| Cd84 | Gria2 | Lamp2 | P2rx7 | Siglecf | Vim |
| Cd86 | Grm3 | Lgmn | Pacsin1 | Sirt1 | sTREM2 |
| Cdk20 | Grn | Lig1 | Pak1 | Slamf9 | sCSFIR |
| Nfl | CXCL10 | IL-1β | IL-6 | IL-8 | TNF-α |
| IGF1 | SPP1/osteopontin | IFN-γ | IL-10 | IL-12P70 | IL-22 |
| IL-4 | CXCL11/ITAC | chitotriosidase | IL-5 | IFN-a4 | IFN-β |
| LIF | MIP3B/CCL19 | TNF | CRP | IL-20 | IL-33 |
| IL-12 | CSF2/GMCSF | OSM | CNTF | CCL4 | IL-11 |
| CST7 | IL-17 | IL-18 | CCL2 | CCL21 | CCL3 |
| CD86 | CH25H | CCR7 | FasL | CSF2 | CD83 |
| CD206 | MCP-1 | CCL8 | Chi313 | Fzd1 | FIZZ1 |
| IL-12p40 | Arg1 | Ym1 | IL-1RA | IL-27 | IL-34 |
| Apoe | CSF3 | ABCD1 | C1qc | Cd74 | HMGB1 |
| Irf7 | C1qa | C1qb | Tmem119 | P2ry12 | Ifitm3 |
| Mr1 | Olfml3 | | | | |

In some embodiments, a biomarker useful in the methods of the present disclosure is one or more of those listed in Table A**:

TABLE A**
Selected Genes and proteins

| | | | | | |
|---|---|---|---|---|---|
| Nfl | CXCL10 | IL-1β | IL-6 | IL-8 | TNF-α |
| IGF1 | SPP1/ | IFN-γ | IL-10 | IL-12P70 | IL-22 |
| | osteopontin | | | | |
| IL-4 | CXCL11/ | chito- | IL-5 | IFN-a4 | IFN-B |
| | ITAC | triosidase | | | |
| LIF | MIP3B/ | TNF | CRP | IL-20 | IL-33 |
| | CCL19 | | | | |
| IL-12 | CSF2/ | OSM | CNTF | CCL4 | IL-11 |
| | GMCSF | | | | |
| CST7 | IL-17 | IL-18 | CCL2 | CCL21 | CCL3 |
| CD86 | CH25H | CCR7 | FasL | CSF2 | CD83 |
| CD206 | MCP-1 | CCL8 | Chi313 | Fzd1 | FIZZ1 |
| IL-12p40 | Arg1 | Ym1 | IL-1RA | IL-27 | IL-34 |
| Apoe | CSF3 | ABCD1 | C1qc | Cd74 | HMGB1 |
| Irf7 | C1qa | C1qb | P2ry12 | sTREM2 | Ifitm3 |
| SCSFIR | Mr1 | Olfml3 | Tmem119 | | |

In some embodiments, a biomarker useful in the methods of the present disclosure is one or more of those listed in Table B:

TABLE B
Selected Genes

Casp8
Ddx58
Dock2
Fcer1g
Fcgr1
Fcrls
Ifi30
Ifitm3
Itgb5
Mcm5
Msn
Pcna
Pla2g4a
Pole
Psmb8
Ptpn6
Rad51
Slamf9
Tgfb1
Tgfbr1
Top2a
Trp73
Tyrobp

In some embodiments, a biomarker useful in the methods of the present disclosure is one or more of those listed in Table C:

TABLE C
Selected genes that are upregulated by TREM2 agonists

Apex1
Atg14
Atp6v0e
Axl
Bad
Bard1
Bcl2a1a
Birc5
Blnk
Bnip3
Bnip3l
Btk
C3
C3ar1
C4a
C5ar1
Casp4
Casp6
Casp7
Casp8
Ccr5
Cd14
Cd68
Cd72
Cd83
Cd84
Cd86
Cdk20
Cdkn1c
Cflar
Cks1b
Clic4
Cnn2
Csf1
Csf1r
Csf2rb
Ctss
Cx3cr1
Dab2
Ddx58
Dna2
Dock1
Dock2
E2f1
Eed
Emcn
Ercc2
Ezh2
Fcer1g
Fcgr1
Fcrls
Fen1
Gbp2
Gna15
Gpr183
Gpr34
Gria2
Grn
Gsn
Gstm1
Hdac4
Hells
Hpgds
Ifi30
Ifih1
Ifitm2
Ifnar2
Il10rb
Il1a
Il1rl2
Il2rg
Inpp5d
Irak1
Irf1
Irf3
Irf8
Itgam
Itgav
Itgb5
Jun
Kat2b
Kdm5c
Lacc1
Lair1
Lamp1
Lamp2
Lgmn
Lig1
Lilrb4a
Lmnb1
Ltbr
Lyn
Man2b1
Map1lc3a
Mavs
Mb21d1
Mbd2
Mcm2
Mcm5
Mcm6
Mdm2
Mmp14
Mpeg1
Msh2
Msn
Mvp
Ncaph
Ncf1
Nfkb1
Nfkb2
Nqo1
Osmr
P2rx7
Pcna
Pdpn
Pik3cb
Pla2g4a
Pla2g5
Plcg2
Plxdc2
Pole
Prdx1
Prkcq
Pros1
Psmb8
Ptpn6
Ptprc
Rac2
Rad1
Rad51
Rblcc1
Rela
Rnf8
Rp128
Rp136al
Rps2
Rps9
Rrm2
Rsad2
S1pr3
Sall1
Serpina3n
Siglecf
Sirt1
Slamf9
Slco2b1
Slfn8
Smc1a
Sox9
Srgn
Stat1
Stx18
Suz12
Tcirg1
Tgfa
Tgfb1
Tgfbr1
Tlr4
Tm4sf1
Tmem173
Tmem206
Tmem37
Tmem64
Tnfrsf12a
Tnfrsf1a
Top2a
Traf1
Traf2
Trim47
Trp73
Txnrd1
Tyrobp
Vav1
Vim

In some embodiments, a biomarker useful in the methods of the present disclosure is one or more of those listed in Table C*:

TABLE C*
Selected genes and proteins that are upregulated by TREM2 agonists

| | | | | | |
|---|---|---|---|---|---|
| Apex1 | Cflar | Gsn | Lig1 | Pla2g4a | Slco2b1 |
| Atg14 | Cks1b | Gstm1 | Lilrb4a | Pla2g5 | Slfn8 |
| Atp6v0e | Clic4 | Hdac4 | Lmnb1 | Plcg2 | Smc1a |
| Axl | Cnn2 | Hells | Ltbr | Plxdc2 | Sox9 |
| Bad | Csf1 | Hpgds | Lyn | Pole | Srgn |
| Bard1 | Csf1r | Ifi30 | Man2b1 | Prdx1 | Stat1 |
| Bcl2a1a | Csf2rb | Ifih1 | Map1lc3a | Prkcq | Stx18 |
| Birc5 | Ctss | Ifitm2 | Mavs | Pros1 | Suz12 |
| Blnk | Cx3cr1 | Ifnar2 | Mb21d1 | Psmb8 | Tcirg1 |
| Bnip3 | Dab2 | Il10rb | Mbd2 | Ptpn6 | Tgfa |
| Bnip3l | Ddx58 | Il1a | Mcm2 | Ptprc | Tgfb1 |
| Btk | Dna2 | Il1rl2 | Mcm5 | Rac2 | Tgfbr1 |
| C3 | Dock1 | Il2rg | Mcm6 | Rad1 | Tlr4 |
| C3ar1 | Dock2 | Inpp5d | Mdm2 | Rad51 | Tm4sf1 |
| C4a | E2f1 | Irak1 | Mmp14 | Rb1cc1 | Tmem173 |
| C5ar1 | Eed | Irf1 | Mpeg1 | Rela | Tmem206 |
| Casp4 | Emcn | Irf3 | Msh2 | Rnf8 | Tmem37 |
| Casp6 | Ercc2 | Irf8 | Msn | Rpl28 | Tmem64 |
| Casp7 | Ezh2 | Itgam | Mvp | Rpl36al | Tnfrsf12a |
| Casp8 | Fcer1g | Itgav | Ncaph | Rps2 | Tnfrsf1a |
| Ccr5 | Fcgr1 | Itgb5 | Ncf1 | Rps9 | Top2a |
| Cd14 | Fcrls | Jun | Nfkb1 | Rrm2 | Traf1 |
| Cd68 | Fen1 | Kat2b | Nfkb2 | Rsad2 | Traf2 |
| Cd72 | Gbp2 | Kdm5c | Nqo1 | S1pr3 | Trim47 |
| Cd83 | Gna15 | Lacc1 | Osmr | Sall1 | Trp73 |
| Cd84 | Gpr183 | Lair1 | P2rx7 | Serpina3n | Txnrd1 |
| Cd86 | Gpr34 | Lamp1 | Pcna | Siglecf | Tyrobp |
| Cdk20 | Gria2 | Lamp2 | Pdpn | Sirt1 | Vav1 |
| Cdkn1c | Grn | Lgmn | Pik3cb | Slamf9 | Vim |
| sTREM2 | sCSF1R | IL-12p70 | IL-6 | IL-10 | CCL2 |
| CCL4 | CXCL10 | CCL3 | CST7 | CD83 | CD86 |
| IL-4 | CCL8 | FasL | CSF2 | FIZZ1 | CD206 |
| Arg1 | Ym1 | IGF-1 | Chi313 | Fzd1 | IL-34 |

| | | |
|---|---|---|
| IL-1RA | Osteopontin/SPP1 | CCR7 |

In some embodiments, a biomarker useful in the methods of the present disclosure is one or more of those listed in Table D:

TABLE D
Selected genes that are downregulated by TREM2 agonists

Adamts16
AI464131
Arc
Bcl2
Cables1
Camk4
Cd40
Clstn1
Dusp7
Fscn1
Gal3st1
Grm3
Hdac1
Homer1
Iqsec1
Kcnj10
Kdm4d
Lingo1
Ly6a
Map3k1
Mapt
Mertk
Ngf
Nlgn2
Nostrin
Pacsin1
Pak1
Slc6a1
Pik3r2
Tgm2
Tie1
Rab6b
Rapgef3
Tubb3

In some embodiments, a biomarker useful in the methods of the present disclosure is one or more of those listed in Table D*:

TABLE D*
Selected genes and proteins that are downregulated by TREM2 agonists

| | | | | | |
|---|---|---|---|---|---|
| Adamts16 | Cd40 | Hdac1 | Ly6a | Nostrin | Tie1 |
| AI464131 | Clstn1 | Homer1 | Map3k1 | Pacsin1 | Rab6b |
| Arc | Dusp7 | Iqsec1 | Mapt | Pak1 | Rapgef3 |
| Bcl2 | Fscn1 | Kcnj10 | Mertk | Slc6a1 | Tubb3 |
| Cables1 | Gal3st1 | Kdm4d | Ngf | Pik3r2 | IFN-a4 |
| Camk4 | Grm3 | Lingo1 | Nlgn2 | Tgm2 | IFN-β |
| IL-6 | IL-12 p70 | IL-1β | TNF | TNF-α | IL-10 |
| IL-8 | CRP | TGF-β | IL-20 | IL-33 | LIF |
| IFN-γ | OSM | CNTF | CSF2/ | IL-11 | IL-12 |
| | | | GMCSF | | |
| IL-17 | IL-18 | IL-12 p40 | IL-27 | CSF3 | CCR5 |
| ABCD1 | CH25H | | | | |

In some embodiments, the biomarker useful in the methods of the present disclosure is a gene or a protein or variant thereof encoded by a gene, selected from those in any one of Table A, Table A*, Table A**, Table B, Table C, Table C*, Table D, or Table D*.

In some embodiments, the biomarker is a gene selected from those in Table A. In some embodiments, the biomarker is a gene selected from those in Table B. In some embodiments, the biomarker is a gene selected from those in Table C. In some embodiments, the biomarker is a gene selected from those in Table D. In some embodiments, the biomarker is a protein or variant thereof encoded by a gene selected from those in Table A. In some embodiments, the biomarker is a protein or variant thereof encoded by a gene selected from those in Table B. In some embodiments, the biomarker is a protein or variant thereof encoded by a gene selected from those in Table C. In some embodiments, the biomarker is a protein or variant thereof encoded by a gene selected from those in Table D. In some embodiments, the biomarker is a gene selected from those in Table A*. In some embodiments, the biomarker is a gene selected from those in Table C*. In some embodiments, the biomarker is a gene selected from those in Table D*. In some embodiments, the biomarker is a protein or variant thereof encoded by a gene selected from those in Table A*. In some embodiments, the biomarker is a protein or variant thereof encoded by a gene selected from those in Table C*. In some embodiments, the biomarker is a protein or variant thereof encoded by a gene selected from those in Table D*.

In some embodiments, the biomarker is the IL1-RN gene or a protein or variant thereof encoded by the IL1-RN gene, which can be IL1-RA. In some embodiments, the biomarker is the Casp8 gene or a protein or variant thereof encoded by the Casp8 gene. In some embodiments, the biomarker is the Ddx58 gene or a protein or variant thereof encoded by the Ddx58 gene. In some embodiments, the biomarker is the Dock2 gene or a protein or variant thereof encoded by the Dock2 gene. In some embodiments, the biomarker is the Fcer1g gene or a protein or variant thereof encoded by the Fcer1g gene. In some embodiments, the biomarker is the Fcgr1 gene or a protein or variant thereof encoded by the Fcgr1 gene. In some embodiments, the biomarker is the Fcr1s gene or a protein or variant thereof encoded by the Fcr1s gene. In some embodiments, the biomarker is the Ifi30 gene or a protein or variant thereof encoded by the Ifi30 gene. In some embodiments, the biomarker is the Ifitm3 gene or a protein or variant thereof encoded by the Ifitm3 gene. In some embodiments, the biomarker is the Itgb5 gene or a protein or variant thereof encoded by the Itgb5 gene. In some embodiments, the biomarker is the Mcm5 gene or a protein or variant thereof encoded by the Mcm5 gene. In some embodiments, the biomarker is the Msn gene or a protein or variant thereof encoded by the Msn gene. In some embodiments, the biomarker is the Pena gene or a protein or variant thereof encoded by the Pena gene. In some embodiments, the biomarker is the Pla2g4a gene or a protein or variant thereof encoded by the Pla2g4a gene. In some embodiments, the biomarker is the Pole gene or a protein or variant thereof encoded by the Pole gene. In some embodiments, the biomarker is the Psmb8 gene or a protein or variant thereof encoded by the Psmb8 gene. In some embodiments, the biomarker is the Ptpn6 gene or a protein or variant thereof encoded by the Ptpn6 gene. In some embodiments, the biomarker is the Rad51 gene or a protein or variant thereof encoded by the Rad51 gene. In some embodiments, the biomarker is the Slamf9 gene or a protein or variant thereof encoded by the Slamf9 gene. In some embodiments, the biomarker is the Tgfb1 gene or a protein or variant thereof encoded by the Tgfb1 gene. In some embodiments, the biomarker is the Tgfbr1 gene or a protein or variant thereof encoded by the Tgfbr1 gene. In some embodiments, the biomarker is the Top2a gene or a protein or variant thereof encoded by the Top2a gene. In some embodiments, the biomarker is the Trp73 gene or a protein or variant thereof encoded by the Trp73 gene. In some embodiments, the biomarker is the Tyrobp gene or a protein or variant thereof encoded by the Tyrobp gene.

It is contemplated that in certain aspects of the invention, one or more biomarkers from the same class or different class of biomarkers (i.e., gene biomarkers and microglial cell state biomarkers) may be used alone, or in any combination therewith each other as a biomarker panel when used in a method described herein. In some embodiments, a biomarker panel comprises one or more biomarkers selected from those in Table A. In some embodiments, the biomarker panel comprises one or more biomarkers selected from those in Table A*. In some embodiments, the biomarker panel comprises one or more biomarkers selected from those in Table A**. In some embodiments, the biomarker panel comprises one or more biomarkers selected from those in Table B. In some embodiments, the biomarker panel comprises one or more biomarkers selected from those in Table A, and further comprises one or more biomarkers selected from those in Table A**. In some embodiments, the biomarker panel comprises one or more biomarkers selected from those in Table A, and further comprises CXCL10. In some embodiments, the biomarker panel comprises one or more biomarkers selected from those in Table B, and further comprises one or more biomarkers selected from those in Table A**. In some embodiments, the biomarker panel comprises one or more biomarkers selected from those in Table B, and further comprises CXCL10. In some embodiments, the biomarker panel comprises two or more biomarkers selected from those in any one of Table A, Table A*, Table A**, Table B, Table C, Table C*, Table D, or Table D*. In some embodiments, the biomarker panel comprises three or more biomarkers selected from those in any one of Table A, Table A*, Table A**, Table B, Table C, Table C*, Table D, or Table D*. In some embodiments, the biomarker panel comprises four or more biomarkers selected from those in any one of Table A, Table A*, Table A**, Table B, Table C, Table C*, Table D, or Table D*. In some embodiments, the biomarker panel comprises five or more biomarkers selected from those in any one of Table A, Table A*, Table A**, Table B, Table C, Table C*, Table D, or Table D*. In some embodiments, the biomarker panel comprises six or more biomarkers selected from those in any one of Table A, Table A*, Table A**, Table B, Table C, Table C*, Table D, or Table D*. In some embodiments, the biomarker panel comprises seven or more biomarkers selected from those in any one of Table A, Table A*, Table A**, Table B, Table C, Table C*, Table D, or Table D*. In some embodiments, the biomarker panel comprises eight or more biomarkers selected from those in any one of Table A, Table A*, Table A**, Table B, Table C, Table C*, Table D, or Table D*.

In some embodiments, the biomarker panel comprises NFL. In some embodiments, the biomarker panel comprises sCSF1R. In some embodiments, the biomarker panel comprises sTREM2. In some embodiments, the biomarker panel comprises SPP1. In some embodiments, the biomarker panel comprises IL-IRA. In some embodiments, the biomarker panel comprises CSF2. In some embodiments, the biomarker panel comprises Chitotriosidate. In some embodiments, the biomarker panel comprises IP10 (CXCL10).

In some embodiments, the biomarker panel comprises NFL and sTREM2. In some embodiments, the biomarker panel comprises NFL and sCSF1R. In some embodiments, the biomarker panel comprises sCSF1R and sTREM2. In some embodiments, the biomarker panel comprises NFL, sCSF1R and sTREM2. In some embodiments, the biomarker panel comprises sCSF1R and one or more of SPP1, IL-1RA, CSF2, Chitotriosidate and IP10 (CXCL10). In some embodiments, the biomarker panel comprises sTREM2 and one or more of SPP1, IL-1RA, CSF2, Chitotriosidate and IP10 (CXCL10). In some embodiments, the biomarker panel comprises NFL and one or more of SPP1, IL-1RA, CSF2, Chitotriosidate and IP10 (CXCL10). In some embodiments, the biomarker panel comprises sCSF1R and sTREM2 and one or more of SPP1, IL-1RA, CSF2, Chitotriosidate and IP10 (CXCL10). In some embodiments, the biomarker panel comprises NFL and sTREM2 and one or more of SPP1, IL-1RA, CSF2, Chitotriosidate and IP10 (CXCL10). In some embodiments, the biomarker panel comprises NFL and sCSF1R and one or more of SPP1, IL-1RA, CSF2, Chitotriosidate and IP10 (CXCL10). In some embodiments, the biomarker panel comprises NFL, sCSF1R, SPP1, IL-1RA, CSF2, Chitotriosidate and IP10 (CXCL10). In some embodiments, the biomarker panel comprises NFL, sTREM2, SPP1, IL-1RA, CSF2, Chitotriosidate and IP10 (CXCL10). In some embodiments, the biomarker panel comprises sCSF1R, sTREM2, SPP1, IL-1RA, CSF2, Chitotriosidate and IP10 (CXCL10). In some embodiments, the biomarker panel comprises two or more of NFL, sCSF1R, sTREM2, SPP1, IL-1RA, CSF2, Chitotriosidate and IP10 (CXCL10). In some embodiments, the biomarker panel comprises three or more of NFL, sCSF1R, sTREM2, SPP1, IL-1RA, CSF2, Chitotriosidate and IP10 (CXCL10). In some embodiments, the biomarker panel comprises four or more of NFL, sCSF1R, sTREM2, SPP1, IL-1RA, CSF2, Chitotriosidate and IP10 (CXCL10). In some embodiments, the biomarker panel comprises five or more of NFL, sCSF1R, sTREM2, SPP1, IL-1RA, CSF2, Chitotriosidate and IP10 (CXCL10). In some embodiments, the biomarker panel comprises six or more of NFL, sCSF1R, sTREM2, SPP1, IL-1RA, CSF2, Chitotriosidate and IP10 (CXCL10). In some embodiments, the biomarker panel comprises seven or more of NFL, sCSF1R, sTREM2, SPP1, IL-1RA, CSF2, Chitotriosidate and IP10 (CXCL10). In some embodiments, the biomarker panel comprises NFL, sCSF1R, sTREM2, SPP1, IL-IRA, CSF2, Chitotriosidate and IP10 (CXCL10).

In some embodiments, the biomarker panel comprises SPP1, IL-IRA, CSF2, Chitotriosidate and IP10 (CXCL10). In some embodiments, the biomarker panel comprises IL-IRA, CSF2, Chitotriosidate and IP10 (CXCL10). In some embodiments, the biomarker panel comprises SPP1, CSF2, Chitotriosidate and IP10 (CXCL10). In some embodiments, the biomarker panel comprises SPP1, IL-1RA, Chitotriosidate and IP10 (CXCL10). In some embodiments, the biomarker panel comprises SPP1, IL-1RA, CSF2, and IP10 (CXCL10). In some embodiments, the biomarker panel comprises SPP1, IL-1RA, CSF2, and Chitotriosidate. In some embodiments, the biomarker panel is a panel disclosed in this paragraph, further comprising NFL. In some embodiments, the biomarker panel is a panel disclosed in this paragraph, further comprising sCSF1R. In some embodiments, the biomarker panel is a panel disclosed in this paragraph, further comprising sTREM2. In some embodiments, the biomarker panel is a panel disclosed in this paragraph, further comprising a biomarker of Table A. In some embodiments, the biomarker panel is a panel disclosed in this paragraph, further comprising a biomarker of Table A*. In some embodiments, the biomarker panel is a panel disclosed in this paragraph, further comprising a biomarker of Table A**. In some embodiments, the biomarker panel is a panel disclosed in this paragraph, further comprising a biomarker of Table B.

In some embodiments, the expression level of one or more of the above biomarkers are increased after administration of a TREM2 agonist. In some embodiments, the expression level of one or more of the above biomarkers are decreased after administration of a TREM2 agonist.

In some embodiments, one, two, three, four, or five of the one or more biomarkers selected from those in Table A are increased after administration of a TREM2 agonist. In some embodiments, one, two, three, four, or five of the one or more biomarkers selected from those in Table A* are increased after administration of a TREM2 agonist. In some embodiments, one, two, three, four, or five of the one or more biomarkers selected from those in Table B are increased after administration of a TREM2 agonist. In some embodiments, one, two, three, four, or five of the one or more biomarkers selected from those in Table C are increased after administration of a TREM2 agonist. In some embodiments, one, two, three, four, or five of the one or more biomarkers selected from those in Table C* are increased after administration of a TREM2 agonist. In certain embodiments, the TREM2 agonist is an anti-human TREM2 antibody. In certain embodiments, the TREM2 agonist is a small molecule TREM2 agonist.

In some embodiments, one, two, three, four, or five of the one or more biomarkers selected from those in Table A are decreased after administration of a TREM2 agonist. In some embodiments, one, two, three, four, or five of the one or more biomarkers selected from those in Table A* are decreased after administration of a TREM2 agonist. In some embodiments, one, two, three, four, or five of the one or more biomarkers selected from those in Table B are decreased after administration of a TREM2 agonist. In some embodiments, one, two, three, four, or five of the one or more biomarkers selected from those in Table D are decreased after administration of a TREM2 agonist. In some embodiments, one, two, three, four, or five of the one or more biomarkers selected from those in Table D* are decreased after administration of a TREM2 agonist. In certain embodiments, the TREM2 agonist is an anti-human TREM2 antibody. In certain embodiments, the TREM2 agonist is a small molecule TREM2 agonist.

In some embodiments, the increase or decrease in the level of a biomarker in a patient is a measurable increase or decrease that correlates with an increased (or decreased, as the case may be) likelihood of therapeutic benefit for the patient, or for a group of patients, or a patient or group of patients yet to be selected. In some embodiments, the increase or decrease is a statistically significant increase or decrease. The term “statistical significance” is well-known in the art and may be determined using methods known in the art, such as those described herein. In some embodiments, statistical significance means, e.g., p<0.1, p<0.05, p<0.04, p<0.03, p<0.02, or p<0.01 relative to baseline.

In some embodiments, the increase or decrease in the level of a biomarker is observed after the patient has completed one cycle of treatment. In some embodiments, the increase or decrease is observed after two or more cycles of treatment, such as three, four, five, six, seven, eight, nine, or 10 or more cycles. The term “cycle of treatment” is well-known in the art and refers to a physician-defined treatment regimen followed by a patient for a period of time such as 1, 2, 3, or 4 weeks, optionally followed by a period of, e.g., 1, 2, 3, or 4 weeks of patient recovery and/or disease progression monitoring, during which, in some cases, a lower dose of therapeutic agent (or no therapeutic agent at all) is administered. In some embodiments, a cycle of treatment refers to administering a TREM2 agonist, such as hT2AB described herein or a pharmaceutically acceptable salt thereof, either as a monotherapy, or in combination with another therapy.

### 7.4. Methods of the Invention

Certain aspects of the present invention provide a molecule that increases activity of TREM2 (i.e., a TREM2 agonist) for use in treating, preventing, or ameliorating the risk of developing conditions associated with microglial dysfunction in a patient in need thereof.

In some embodiments, conditions or disorders associated with microglial dysfunction are ones that are associated with TREM2 deficiency or loss of TREM2 function. Conditions or disorders associated with TREM2 deficiency or loss of TREM2 function that may be prevented, treated, or ameliorated according to the methods of the invention include, but are not limited to, Nasu-Hakola disease, Alzheimer's disease, frontotemporal dementia, multiple sclerosis, Guillain-Barre syndrome, amyotrophic lateral sclerosis (ALS), Parkinson's disease, traumatic brain injury, spinal cord injury, systemic lupus erythematosus, rheumatoid arthritis, prion disease, stroke, osteoporosis, osteopetrosis, osteosclerosis, skeletal dysplasia, dysosteoplasia, Pyle disease, cerebral autosomal dominant arteriopathy with subcortical infarcts and leukoencephalopathy, cerebral autosomal recessive arteriopathy with subcortical infarcts and leukoencephalopathy, cerebroretinal vasculopathy, or metachromatic leukodystrophy.

In some embodiments, conditions or disorders associated with microglial dysfunction are ones that are associated with dysfunction of colony stimulating factor 1 receptor (CSF1R, also known as macrophage colony-stimulating factor receptor/M-CSFR, or cluster of differentiation 115/CD115). Conditions or disorders associated with dysfunction of CSF1R that may be prevented, treated, or ameliorated according to the methods of the invention include, but are not limited to, adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS). In some embodiments, conditions or disorders associated with dysfunction of CSF1R also include Nasu-Hakola disease, Alzheimer's disease, frontotemporal dementia, multiple sclerosis, Guillain-Barre syndrome, amyotrophic lateral sclerosis (ALS), Parkinson's disease, traumatic brain injury, spinal cord injury, systemic lupus erythematosus, rheumatoid arthritis, prion disease, stroke, osteoporosis, osteopetrosis, osteosclerosis, skeletal dysplasia, dysosteoplasia, Pyle disease, cerebral autosomal dominant arteriopathy with subcortical infarcts and leukoencephalopathy, cerebral autosomal recessive arteriopathy with subcortical infarcts and leukoencephalopathy, cerebroretinal vasculopathy, or metachromatic leukodystrophy wherein any of the aforementioned diseases or disorders are present in a patient exhibiting CSF1R dysfunction, or having a mutation in a gene affecting the function of CSF1R.

In some embodiments, conditions or disorders associated with microglial dysfunction are ones that are associated with dysfunction of ATP-binding cassette transporter 1 (ABCD1). Conditions or disorders associated with dysfunction of ABCD1 that may be prevented, treated, or ameliorated according to the methods of the invention include, but are not limited to, X-linked adrenoleukodystrophy (x-ALD), childhood cerebral adrenoleukodystrophy (cALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX). In some embodiments, conditions or disorders associated with dysfunction of ABCD1 also include Nasu-Hakola disease, Alzheimer's disease, frontotemporal dementia, multiple sclerosis, Guillain-Barre syndrome, amyotrophic lateral sclerosis (ALS), Parkinson's disease, traumatic brain injury, spinal cord injury, systemic lupus erythematosus, rheumatoid arthritis, prion disease, stroke, osteoporosis, osteopetrosis, osteosclerosis, skeletal dysplasia, dysosteoplasia, Pyle disease, cerebral autosomal dominant arteriopathy with subcortical infarcts and leukoencephalopathy, cerebral autosomal recessive arteriopathy with subcortical infarcts and leukoencephalopathy, cerebroretinal vasculopathy, or metachromatic leukodystrophy wherein any of the aforementioned diseases or disorders are present in a patient exhibiting ABCD1 dysfunction, or having a mutation in a gene affecting the function of ABCD1.

In some embodiments, the condition or disorder associated with microglial dysfunction is autism or an autism spectrum disorder (ASD). In some embodiments, the condition or disorder is autism. In some embodiments, the condition or disorder is Asperger syndrome.

In one aspect, the present invention provides a method of identifying a patient with a condition or disorder associated with microglial dysfunction who will benefit from treatment with a TREM2 agonist, comprising:
(a) obtaining a first biological sample from the patient prior to administration of the TREM2 agonist to the patient;
(b) measuring a level in the first biological sample of one or more biomarkers selected from those in Table A;
(c) administering to the patient an effective amount of the TREM2 agonist;
(d) obtaining a second biological sample from the patient after administration of the TREM2 agonist to the patient; and
(e) measuring a level in the second biological sample of one or more biomarkers selected from those in Table A.

In certain embodiments, the method further comprises additional administration of the TREM2 agonist to the patient, if the patient is determined to benefit from treatment with the TREM2 agonist. In certain embodiments of the above method, the one or more biomarkers are selected from those in Table A*, instead of Table A. In certain embodiments of the above method, the one or more biomarkers are a panel of biomarkers disclosed herein, instead of being selected from those in Table A. In certain embodiments, the one or more biomarkers are selected from those in Table B. In certain embodiments, the TREM2 agonist is an anti-hTREM2 antibody. In certain embodiments, the TREM2 agonist is a small molecule TREM2 agonist.

In another aspect, the present invention provides a method of identifying a patient with a condition or disorder associated with microglial dysfunction who is likely to benefit, or has an increased probability of benefitting relative to an otherwise similar patient, from treatment with a TREM2 agonist, comprising:
(a) obtaining a first biological sample from the patient prior to administration of the TREM2 agonist to the patient;
(b) measuring a level in the first biological sample of one or more biomarkers selected those in Table A;
(c) administering to the patient an effective amount of the TREM2 agonist;
(d) obtaining a second biological sample from the patient after administration of the TREM2 agonist to the patient; and
(e) measuring a level in the second biological sample of one or more biomarkers selected from those in Table A;
   wherein the response of the condition or disorder to step (c) is predictive of the likelihood of successful treatment of the condition or disorder associated with microglial dysfunction based on a greater or lesser response of the condition or disorder associated with microglial dysfunction compared with one or more similar patients and as evaluated using one or more of the biomarkers.

In certain embodiments of the above method, the one or more biomarkers are selected from those in Table A*, instead of Table A. In certain embodiments of the above method, the one or more biomarkers are a panel of biomarkers disclosed herein, instead of being selected from those in Table A. In certain embodiments, the one or more biomarkers are selected from those in Table B. In certain embodiments, the TREM2 agonist is an anti-hTREM2 antibody. In certain embodiments, the TREM2 agonist is a small molecule TREM2 agonist.

In another aspect, the present invention provides a method of assaying a biological sample taken from a patient in vitro or ex vivo to determine if the condition or disorder associated with microglial dysfunction in the patient will respond, or has an increased probability of responding, to treatment with a TREM2 agonist, comprising:
(a) obtaining a first biological sample from the patient prior to administration of the TREM2 agonist to the patient;
(b) measuring a level in the first biological sample of one or more biomarkers selected from those in Table A; and
(c) administering to the patient an effective amount of the TREM2 agonist, if the patient with the condition or disorder will respond, or has an increased probability of responding, to treatment with a TREM2 agonist.

In certain embodiments of the above method, the one or more biomarkers are selected from those in Table A*, instead of Table A. In certain embodiments of the above method, the one or more biomarkers are a panel of biomarkers disclosed herein, instead of being selected from those in Table A. In certain embodiments, the one or more biomarkers are selected from those in Table B. In certain embodiments, the TREM2 agonist is an anti-hTREM2 antibody. In certain embodiments, the TREM2 agonist is a small molecule TREM2 agonist.

In another aspect, the present invention provides a method of treating a condition or disorder associated with microglial dysfunction in a patient who either does not respond to prior treatment or whose condition or disorder has become refractory after initially responding to prior treatment, comprising:
(a) obtaining a first biological sample from the patient prior to administration of the TREM2 agonist to the patient;
(b) measuring a level in the first biological sample of one or more biomarkers selected from those in Table A;
(c) administering to the patient an effective amount of the TREM2 agonist, if the patient with the condition or disorder associated with microglial dysfunction will respond, or has an increased probability of responding, to treatment with the TREM2 agonist;
(d) obtaining a second biological sample after administration of the TREM2 agonist to the patient; and
(e) measuring a level in the second biological sample of one or more biomarkers selected from those in Table A;
   wherein the response of the condition or disorder to step (c) is predictive of the likelihood of successful treatment of the condition or disorder associated with microglial dysfunction based on a greater or lesser response of the condition or disorder associated with microglial dysfunction compared with one or more similar patients and as evaluated using one or more of the biomarkers.

In certain embodiments of the above method, the one or more biomarkers are selected from those in Table A*, instead of Table A. In certain embodiments of the above method, the one or more biomarkers are a panel of biomarkers disclosed herein, instead of being selected from those in Table A. In certain embodiments, the one or more biomarkers are selected from those in Table B. In certain embodiments, the TREM2 agonist is an anti-hTREM2 antibody. In certain embodiments, the TREM2 agonist is a small molecule TREM2 agonist.

In another aspect, the present invention provides a method of predicting whether a condition or disorder associated with microglial dysfunction will respond to treatment with a second treatment for the condition or disorder following treatment with a TREM2 agonist, comprising:
(a) obtaining a first biological sample from the patient prior to administration of the TREM2 agonist to the patient;
(b) measuring a level in the first biological sample of one or more biomarkers selected from those in Table A;
(c) administering to the patient an effective amount of the TREM2 agonist;
(d) obtaining a second biological sample after administration of the TREM2 agonist to the patient; and
(e) measuring a level in the second biological sample of one or more biomarkers selected from those in Table A;
   wherein the response of the condition or disorder to step (c) is predictive of the likelihood of successful treatment of the condition or disorder based on a greater or lesser response of the condition or disorder compared with one or more similar patients and as evaluated using one or more of the biomarkers.

In certain embodiments of the above method, the one or more biomarkers are selected from those in Table A*, instead of Table A. In certain embodiments of the above method, the one or more biomarkers are a panel of biomarkers disclosed herein, instead of being selected from those in Table A. In certain embodiments, the one or more biomarkers are selected from those in Table B. In certain embodiments, the TREM2 agonist is an anti-hTREM2 antibody. In certain embodiments, the TREM2 agonist is a small molecule TREM2 agonist.

In some embodiments, the patient suffers from Alzheimer's disease or a related disorder and treatment with a TREM2 agonist primes the plaque microenvironment such that the plaque becomes more likely to respond to a second therapeutic agent capable of treating conditions and disorders associated with microglial dysfunction. In some embodiments, the plaque does not respond to monotherapy with a single treatment, but becomes primed and responds to the treatment when combined with a TREM2 agonist. In some embodiments, the plaque initially responds to the monotherapy with a therapeutic agent capable of treating conditions and disorders associated with microglial dysfunction, but becomes refractory. In some embodiments, after treatment with a TREM2 agonist, the plaque can be treated effectively with the therapeutic agent capable of treating conditions and disorders associated with microglial dysfunction.

In some embodiments, the above method is useful in the identification of a patient who will benefit from treatment with a TREM2 agonist. Such a patient is characterized in that the level of one or more biomarkers selected from those in Table A is higher or lower in the second biological sample from the patient than in the first biological sample from the patient. In some embodiments, when the level of one or more biomarkers selected from those in Table A is higher in the second biological sample from the patient than in the first biological sample from the patient, then the patient is administered one or more additional doses of the TREM2 agonist, if the one or more biomarkers are one of those that is upregulated by administration of the TREM2 agonist. In some embodiments, when the level of one or more biomarkers selected from those in Table A is lower in the second biological sample from the patient than in the first biological sample from the patient, then the patient is administered one or more additional doses of the TREM2 agonist, if the one or more biomarkers are one of those that is downregulated by administration of the TREM2 agonist. This is because such a patient is considered likely to benefit from continued treatment with the TREM2 agonist, based on the respective increase or decrease in the biomarker levels. In any of the above embodiments, the biomarkers may be one or more biomarkers selected from those in Table A* instead of Table A. In certain embodiments of the above method, the one or more biomarkers are a panel of biomarkers disclosed herein, instead of being selected from those in Table A. Biomarkers that are expected to be upregulated by administration of a TREM2 agonist are those listed in Table C or Table C*. Biomarkers that are expected to be downregulated by administration of a TREM2 agonist are those listed in Table D or Table D*. In certain embodiments, the one or more biomarkers are selected from those in Table B. In certain embodiments, the TREM2 agonist is an anti-hTREM2 antibody. In certain embodiments, the TREM2 agonist is a small molecule TREM2 agonist.

In another aspect, the present invention provides a method of treating a condition or disorder associated with microglial dysfunction with a TREM2 agonist, comprising:
(a) obtaining a first biological sample from the patient prior to administration of the TREM2 agonist to the patient;
(b) measuring a level in the first biological sample of one or more biomarkers selected from those in Table A;
(c) administering to the patient an effective amount of a TREM2 agonist;
(d) obtaining a second biological sample after administration of the TREM2 agonist to the patient; and
(e) measuring a level in the second biological sample of one or more biomarkers selected from those in Table A;
   wherein when the level of one or more biomarkers selected from those in Table A is higher in the second biological sample from the patient than in the first biological sample from the patient, then the patient is administered one or more additional doses of the TREM2 agonist. In certain embodiments of the above method, the one or more biomarkers are selected from those in Table A*, instead of Table A. In certain embodiments of the above method, the one or more biomarkers are a panel of biomarkers disclosed herein, instead of being selected from those in Table A. In certain embodiments, the one or more biomarkers are selected from those in Table C. In certain embodiments, the one or more biomarkers are selected from those in Table C*.

In certain embodiments, the one or more biomarkers are selected from those in Table B. In certain embodiments, the TREM2 agonist is an anti-hTREM2 antibody. In certain embodiments, the TREM2 agonist is a small molecule TREM2 agonist.

In another aspect, the present invention provides a method of treating a condition or disorder associated with microglial dysfunction with a TREM2 agonist, comprising:
(a) obtaining a first biological sample from the patient prior to administration of the TREM2 agonist to the patient;
(b) measuring a level in the first biological sample of one or more biomarkers selected from those in Table A;
(c) administering to the patient an effective amount of a TREM2 agonist;
(d) obtaining a second biological sample after administration of the TREM2 agonist to the patient; and
(e) measuring a level in the second biological sample of one or more biomarkers selected from those in Table A;
   wherein when the level of one or more biomarkers selected from those in Table A is lower in the second biological sample from the patient than in the first biological sample from the patient, then the patient is administered one or more additional doses of the TREM2 agonist. In certain embodiments of the above method, the one or more biomarkers are selected from those in Table A*, instead of Table A. In certain embodiments of the above method, the one or more biomarkers are a panel of biomarkers disclosed herein, instead of being selected from those in Table A. In certain embodiments, the one or more biomarkers are selected from those in Table D. In certain embodiments, the one or more biomarkers are selected from those in Table D*.

In certain embodiments, the one or more biomarkers are selected from those in Table B. In certain embodiments, the TREM2 agonist is an anti-hTREM2 antibody. In certain embodiments, the TREM2 agonist is a small molecule TREM2 agonist.

In another aspect, the present invention provides a method of evaluating a patient response to a TREM2 agonist, comprising the steps of:
(a) obtaining a first biological sample from the patient prior to administration of the TREM2 agonist to the patient;
(b) measuring a level in the first biological sample of one or more biomarkers selected from those in Table A;
(c) administering to the patient an effective amount of a TREM2 agonist;
(d) obtaining a second biological sample after administration of the TREM2 agonist to the patient; and
(e) measuring a level in the second biological sample of one or more biomarkers selected from those in Table A;
   wherein the response of the condition or disorder to step (c) is evaluated to split, classify, or stratify the patient into one of two or more groups based on a greater or lesser response of the plaque compared with one or more similar patients.

In certain embodiments of the above method, the one or more biomarkers are selected from those in Table A*, instead of Table A. In certain embodiments of the above method, the one or more biomarkers are a panel of biomarkers disclosed herein, instead of being selected from those in Table A. In certain embodiments, the one or more biomarkers are selected from those in Table B. In certain embodiments, the TREM2 agonist is an anti-hTREM2 antibody. In certain embodiments, the TREM2 agonist is a small molecule TREM2 agonist.

In another aspect, the present invention provides a method of predicting a patient response to a TREM2 agonist, comprising the steps of:
(a) obtaining a first biological sample from the patient prior to administration of the TREM2 agonist to the patient;
(b) measuring a level in the first biological sample from the patient of one or more biomarkers selected from those in Table A;
(c) administering to the patient an effective amount of the TREM2 agonist;
(d) obtaining a second biological sample from the patient after administration of the TREM2 agonist to the patient; and
(e) measuring a level in the second biological sample from the patient of one or more biomarkers selected from those in Table A;
   wherein the patient's response to step (c) is predictive of the likelihood of successful treatment based on a greater or lesser response of the patient compared with one or more similar patients and as evaluated using one or more of the biomarkers.

In certain embodiments of the above method, the one or more biomarkers are selected from those in Table A*, instead of Table A. In certain embodiments of the above method, the one or more biomarkers are a panel of biomarkers disclosed herein, instead of being selected from those in Table A. In certain embodiments, the one or more biomarkers are selected from those in Table B. In certain embodiments, the TREM2 agonist is an anti-hTREM2 antibody. In certain embodiments, the TREM2 agonist is a small molecule TREM2 agonist.

In another aspect, the present invention provides a method of predicting a treatment response to a TREM2 agonist for a condition or disorder associated with microglial dysfunction in a patient, comprising the steps of:
(a) obtaining a first biological sample from the patient prior to administration of the TREM2 agonist to the patient;
(b) measuring a level in the first biological sample of one or more biomarkers selected from those in Table A;
(c) treating the biological sample from the patient or a reference sample;
(d) measuring a level in the treated biological sample of one or more biomarkers selected from those in Table A;
(e) comparing one of more biomarkers in the pre-treatment biological sample with one or more biomarkers in the treated biological sample or treated reference sample; and
(f) optionally, proceeding with administration of the TREM2 agonist to the patient, if such administration is predicted to have an equivalent or higher likelihood of success relative to an alternative method of treating the condition or disorder associated with microglial dysfunction;
   wherein the biomarker change in response to step (c) is predictive of the likelihood of successful treatment of the condition or disorder associated with microglial dysfunction based on a greater or lesser biomarker change compared with one or more similar patients and as evaluated using one or more of the biomarkers.

In certain embodiments of the above method, the one or more biomarkers are selected from those in Table A*, instead of Table A. In certain embodiments of the above method, the one or more biomarkers are a panel of biomarkers disclosed herein, instead of being selected from those in Table A. In certain embodiments, the one or more biomarkers are selected from those in Table B. In certain embodiments, the TREM2 agonist is an anti-hTREM2 antibody. In certain embodiments, the TREM2 agonist is a small molecule TREM2 agonist.

In some embodiments, the reference sample is from another patient, such as a patient with a similar pathology of the condition or disorder; or the reference sample may be a culture or other in vitro sample of a similar pathology of the condition or disorder.

In another aspect, the present invention provides a method of monitoring a patient response to a TREM2 agonist, comprising the steps of:
(a) obtaining a first biological sample from the patient prior to administration of the TREM2 agonist to the patient;
(b) measuring a level in the first biological sample from the patient of one or more biomarkers selected from those in Table A;
(c) administering to the patient an effective amount of the TREM2 agonist;
(d) obtaining a one or more subsequent biological samples from the patient after administration of the TREM2 agonist to the patient; and
(e) measuring a level in the subsequent biological sample(s) of one or more biomarkers selected from those in Table A;
   wherein the levels of one of more biomarkers in the first biological sample and subsequent biological samples can be compared and changes in one or more of the biomarkers indicate a patient response.

In certain embodiments of the above method, the one or more biomarkers are selected from those in Table A*, instead of Table A. In certain embodiments of the above method, the one or more biomarkers are a panel of biomarkers disclosed herein, instead of being selected from those in Table A. In certain embodiments, the one or more biomarkers are selected from those in Table B. In certain embodiments, the TREM2 agonist is an anti-hTREM2 antibody. In certain embodiments, the TREM2 agonist is a small molecule TREM2 agonist.

In some embodiments, the patient response to a TREM2 agonist is measured once per week or every two weeks. In some embodiments, the patient response is measured once a month. In some embodiments, the patient's response is measured bimonthly. In some embodiments, the patient's response is measured quarterly (once every three months). In some embodiments, the patient's response is measured annually.

In some embodiments, the patient response to a TREM2 agonist is monitored while undergoing treatment. In some embodiments, the patient response is monitored after treatment is concluded.

In another aspect, the present invention provides a method of deriving a biomarker signature that is predictive of response to treatment with a TREM2 agonist, comprising:
(a) obtaining a pre-treatment biological sample from each patient in a patient cohort diagnosed with a condition or disorder associated with microglial dysfunction;
(b) obtaining, for each patient in the cohort, a response value following treatment with the TREM2 agonist;
(c) measuring the raw biomarker levels in each biological sample for each gene in a biomarker platform, wherein the biomarker platform comprises a clinical response biomarker set of those in Table A;
(d) normalizing, for each biological sample, each of the measured raw biomarker levels for the clinical response biomarkers using the measured biomarker levels of a set of normalization biomarkers; and (e) comparing the biomarker levels for all of the biological samples and the response values for all of the patients in the cohort to select a cutoff for the biomarker signature score that divides the patient cohort to meet a target biomarker clinical utility criterion.

In some embodiments, the biomarker platform comprises a gene expression platform that comprises a clinical response gene set. In some embodiments, the method further comprises the steps of:
(f) weighting, for each biological sample and each biomarker, such as a gene in a gene signature of interest, the normalized biomarker (e.g., RNA biomarker) expression levels using a pre-defined multiplication coefficient for that gene;
(g) adding, for each patient, the weighted biomarker (e.g., RNA biomarker) expression levels to generate a biomarker signature score, e.g., a gene signature score, for each patient in the cohort.

In certain embodiments of the above method, the one or more biomarkers are selected from those in Table A*, instead of Table A. In certain embodiments of the above method, the biomarker platform comprises a panel of biomarkers disclosed herein, instead of being selected from those in Table A. In certain embodiments, the clinical response biomarkers are selected from those in Table B. In certain embodiments, the TREM2 agonist is an anti-hTREM2 antibody. In certain embodiments, the TREM2 agonist is a small molecule TREM2 agonist.

In another aspect, the present invention provides a method of testing a biological sample from a patient for the presence or absence of a gene signature biomarker of response of a condition or disorder associated with microglial dysfunction to a TREM2 agonist, comprising:
(a) measuring the raw RNA level in the biological sample for each gene in a gene expression platform, wherein the gene expression platform comprises a clinical response gene set selected from those in Table A and a normalization gene set of housekeeping genes, and optionally wherein about 80%, or about 90%, of the selected clinical response genes exhibit RNA levels that are positively correlated with response to the condition or disorder;
(b) normalizing the measured raw RNA level for each clinical response gene in a pre-defined gene signature for the biological sample using the measured RNA levels of the normalization genes, wherein the pre-defined gene signature consists of at least 2 of the clinical response genes, thus obtaining a gene signature score;
(c) comparing the gene signature score to a reference score for the gene signature; and
(d) classifying the biological sample as biomarker high or biomarker low;
   wherein if the generated score is equal to or greater than the reference score, then the biological sample is classified as biomarker high, and if the generated score is less than the reference score, then the biological sample is classified as biomarker low.

In certain embodiments of the above method, the clinical response biomarkers are selected from those in Table A*, instead of Table A. In certain embodiments of the above method, the gene expression platform comprises a panel of biomarkers disclosed herein, instead of being selected from those in Table A. In certain embodiments, the clinical response biomarkers are selected from those in Table B. In certain embodiments, the TREM2 agonist is an anti-hTREM2 antibody. In certain embodiments, the TREM2 agonist is a small molecule TREM2 agonist.

In some embodiments, after step (b) the method comprises the further steps of:
(i) weighting each normalized RNA value using a pre-defined multiplication co-efficient;
(ii) adding the weighted RNA expression levels to generate a weighted gene signature score.

In some embodiments, the normalization gene set comprises about 1 to 5 housekeeping genes, 5 to 10 housekeeping genes, 10 to about 20 housekeeping genes, or about 30-40 housekeeping genes.

In another aspect, the present invention provides a method of testing a biological sample from a patient diagnosed with a condition or disorder associated with microglial dysfunction for the presence or absence of a biomarker signature of response of the condition or disorder to a TREM2 agonist, comprising:
(a) measuring the raw biomarker level in the biological sample for each biomarker in a biomarker platform, wherein the biomarker platform comprises a clinical response biomarker set selected from those in Table A and a normalization biomarker set, and optionally wherein about 80%, or about 90%, of the clinical response biomarkers exhibit levels that are positively correlated with the condition or disorder response;
(b) normalizing the measured raw biomarker level for each clinical response biomarker in a pre-defined biomarker signature for the biological sample using the measured biomarker levels of the normalization biomarkers, wherein the pre-defined biomarker signature consists of at least 2 of the clinical response biomarkers;
(c) comparing the normalized biomarker levels and a set of reference biomarker levels for the biological sample; and
(d) classifying the biological sample as biomarker high or biomarker low;
   wherein if the normalized biomarker levels are equal to or greater than the reference biomarker levels, then the biological sample is classified as biomarker high, and if the normalized biomarker levels are less than the reference biomarker levels, then the biological sample is classified as biomarker low.

In certain embodiments of the above method, the clinical response biomarkers are selected from those in Table A*, instead of Table A. In certain embodiments of the above method, the biomarker platform comprises a panel of biomarkers disclosed herein, instead of being selected from those in Table A. In certain embodiments, the clinical response biomarkers are selected from those in Table B. In certain embodiments, the TREM2 agonist is an anti-hTREM2 antibody. In certain embodiments, the TREM2 agonist is a small molecule TREM2 agonist.

In some embodiments, the normalization biomarker set comprises about 10 to about 12 housekeeping genes, or about 30-40 housekeeping genes.

In another aspect, the present invention provides a system for testing a sample of a biological sample removed from a patient having a condition or disorder associated with microglial dysfunction for the presence or absence of a biomarker signature of response to a TREM2 agonist, comprising:
(i) a sample analyzer for measuring raw biomarker levels in a biomarker platform, wherein the biomarker platform consists of a set of clinical response biomarkers selected from those in Table A; and a set of normalization biomarkers; and
(ii) a computer program for receiving and analyzing the measured biomarker levels to:
   (a) normalize the measured raw biomarker level for each clinical response biomarker in a pre-defined biomarker signature for the biological sample using the measured levels of the normalization biomarkers;
   (b) compare the generated biomarker level to a reference level for the biomarker signature; and
   (c) classify the biological sample as biomarker high or biomarker low, wherein if the generated score is equal to or greater than the reference score, then the biological sample is classified as biomarker high, and if the generated score is less than the reference score, then the biological sample is classified as biomarker low.

In certain embodiments of the above method, the clinical response biomarkers are selected from those in Table A*, instead of Table A. In certain embodiments of the above method, the biomarker platform comprises a panel of biomarkers disclosed herein, instead of being selected from those in Table A. In certain embodiments, the clinical response biomarkers are selected from those in Table B. In certain embodiments, the TREM2 agonist is an anti-hTREM2 antibody. In certain embodiments, the TREM2 agonist is a small molecule TREM2 agonist.

In another aspect, the present invention provides a system for testing a biological sample from a patient diagnosed with a condition or disorder associated with microglial dysfunction for the presence or absence of a biomarker signature of response of the condition or disorder to a TREM2 agonist, comprising:
(i) a sample analyzer for measuring raw biomarker levels in a biomarker platform, wherein the biomarker platform consists of a set of clinical response biomarkers selected from those in Table A; and a set of normalization biomarkers; and
(ii) a computer program for receiving and analyzing the measured biomarker levels to
   (a) normalize the measured raw biomarker level for each clinical response biomarker in a pre-defined biomarker signature for the biological sample using the measured levels of the normalization biomarkers;
   (b) weight each normalized biomarker level using a pre-defined multiplication coefficient;
   (c) add the weighted biomarker levels to generate a biomarker signature score;
   (d) compare the generated score to a reference score for the biomarker signature; and
   (e) classify the biological sample as biomarker high or biomarker low, wherein if the generated score is equal to or greater than the reference score, then the biological sample is classified as biomarker high, and if the generated score is less than the reference score, then the biological sample is classified as biomarker low.

In certain embodiments of the above method, the clinical response biomarkers are selected from those in Table A*, instead of Table A. In certain embodiments of the above method, the biomarker platform comprises a panel of biomarkers disclosed herein, instead of being selected from those in Table A. In certain embodiments, the clinical response biomarkers are selected from those in Table B. In certain embodiments, the TREM2 agonist is an anti-hTREM2 antibody. In certain embodiments, the TREM2 agonist is a small molecule TREM2 agonist.

In some embodiments, the biomarker comprises the RNA expression level of a gene described herein, such as those in Table A. In some embodiments, the biomarker comprises the RNA expression level of a gene described herein, such as those in Table A*. In some embodiments, the biomarker comprises the RNA expression level of a gene described herein, such as those in Table B.

In another aspect, the present invention provides a kit for assaying a biological sample from a patient suffering from a condition or disorder associated with microglial dysfunction who has been treated with a TREM2 agonist to obtain normalized RNA expression scores for a gene signature associated with said condition or disorder, wherein the kit comprises:
(a) a set of hybridization probes capable of specifically binding to a transcript expressed by each of the genes; and
(b) a set of reagents designed to quantify the number of specific hybridization complexes formed with each hybridization probe.

In some embodiments, the gene signature comprises two or more genes in Table A. In some embodiments, the gene signature comprises two or more genes in Table A*. In some embodiments, the gene signature comprises two or more genes selected from those in a biomarker panel disclosed herein. In certain embodiments, the gene signature comprises two or more genes in Table B. In certain embodiments, the TREM2 agonist is an anti-hTREM2 antibody. In certain embodiments, the TREM2 agonist is a small molecule TREM2 agonist.

In another aspect, the present invention provides a method for treating a patient having a condition or disorder associated with microglial dysfunction, comprising determining if a biological sample from the patient is positive or negative for a biomarker such as a gene signature biomarker and administering to the patient a TREM2 agonist if the biological sample is positive for the biomarker and administering to the subject a treatment for the condition or disorder that does not include a TREM2 agonist if the biological sample from the patient is negative for the biomarker, wherein the biomarker, such as a gene signature biomarker, comprises at least two of the clinical response biomarkers selected from those in Table A. In some embodiments, a multi-gene signature score derived from one or more biomarkers, such as those in Table A, can be used as one “biomarker” in the same grouping as other individual gene biomarkers, to calculate a more predictive gene signature score. In some embodiments, a multi-gene signature score derived from one or more biomarkers, such as those in Table A*, can be used as one “biomarker” in the same grouping as other individual gene biomarkers, to calculate a more predictive gene signature score. In some embodiments, a multi-gene signature score derived from one or more biomarkers, such as those in Table B, can be used as one “biomarker” in the same grouping as other individual gene biomarkers, to calculate a more predictive gene signature score.

In another aspect, the present invention provides a method of testing a biological sample from a patient to generate a signature score for a gene signature that is correlated with response of a condition or disorder associated with microglial dysfunction to a TREM2 agonist, wherein the method comprises:
(a) measuring the raw RNA level in the biological sample for each gene in the gene signature and for each gene in a normalization gene set, wherein the gene signature comprises genes selected from those in Table A;
(b) normalizing the measured raw RNA level for each gene in the gene signature using the measured RNA levels of the normalization genes;
(c) multiplying each normalized RNA value by a calculated scoring weight set to generate a weighted RNA expression value; and
(d) adding the weighted RNA expression values to generate the gene signature score.

In certain embodiments of the above method, the gene signature comprises genes selected from those in Table A*, instead of Table A. In certain embodiments of the above method, the gene signature comprises a panel of biomarkers disclosed herein, instead of being selected from those in Table A. In certain embodiments, the gene signature comprises genes selected from those in Table B. In certain embodiments, the TREM2 agonist is an anti-hTREM2 antibody. In certain embodiments, the TREM2 agonist is a small molecule TREM2 agonist.

In any of the above embodiments, each method can further comprise an additional step of administering to the patient a TREM2 agonist, based on the information gathered by the method. In some embodiments, the dosage amount of the TREM2 agonist can be altered (either increased or decreased) based on the information gathered by the method.

In any of the above embodiments, the selection of genes and/or biomarkers in each method described above can further comprise CXCL10. In any of the above embodiments, the selection of genes and/or biomarkers in each method described above can further comprise those listed in Table A**.

In embodiments of any of the above methods, the biomarkers and genes being measured can be part of a biomarker panel. In some embodiments, the biomarker panel comprises NFL. In some embodiments, the biomarker panel comprises sCSF1R. In some embodiments, the biomarker panel comprises sTREM2. In some embodiments, the biomarker panel comprises SPP1. In some embodiments, the biomarker panel comprises IL-IRA. In some embodiments, the biomarker panel comprises CSF2. In some embodiments, the biomarker panel comprises Chitotriosidate. In some embodiments, the biomarker panel comprises IP10 (CXCL10).

In some embodiments, the biomarker panel comprises NFL and sTREM2. In some embodiments, the biomarker panel comprises NFL and sCSF1R. In some embodiments, the biomarker panel comprises sCSF1R and sTREM2. In some embodiments, the biomarker panel comprises NFL, sCSF1R, and sTREM2. In some embodiments, the biomarker panel comprises sCSF1R and one or more of SPP1, IL-1RA, CSF2, Chitotriosidate, and IP10 (CXCL10). In some embodiments, the biomarker panel comprises sTREM2 and one or more of SPP1, IL-1RA, CSF2, Chitotriosidate, and IP10 (CXCL10). In some embodiments, the biomarker panel comprises NFL and one or more of SPP1, IL-1RA, CSF2, Chitotriosidate, and IP10 (CXCL10). In some embodiments, the biomarker panel comprises sCSF1R and sTREM2, and one or more of SPP1, IL-1RA, CSF2, Chitotriosidate, and IP10 (CXCL10). In some embodiments, the biomarker panel comprises NFL and sTREM2, and one or more of SPP1, IL-1RA, CSF2, Chitotriosidate, and IP10 (CXCL10). In some embodiments, the biomarker panel comprises NFL and sCSF1R, and one or more of SPP1, IL-1RA, CSF2, Chitotriosidate, and IP10 (CXCL10). In some embodiments, the biomarker panel comprises NFL, sCSF1R, SPP1, IL-1RA, CSF2, Chitotriosidate, and IP10 (CXCL10). In some embodiments, the biomarker panel comprises NFL, sTREM2, SPP1, IL-1RA, CSF2, Chitotriosidate, and IP10 (CXCL10). In some embodiments, the biomarker panel comprises sCSF1R, sTREM2, SPP1, IL-IRA, CSF2, Chitotriosidate, and IP10 (CXCL10). In some embodiments, the biomarkerpanel comprises two or more of NFL, sCSF1R, sTREM2, SPP1, IL-IRA, CSF2, Chitotriosidate, and IP10 (CXCL10). In some embodiments, the biomarker panel comprises three or more of NFL, sCSF1R, sTREM2, SPP1, IL-IRA, CSF2, Chitotriosidate, and IP10 (CXCL10). In some embodiments, the biomarker panel comprises four or more of NFL, sCSF1R, sTREM2, SPP1, IL-1RA, CSF2, Chitotriosidate, and IP10 (CXCL10). In some embodiments, the biomarker panel comprises five or more of NFL, sCSF1R, sTREM2, SPP1, IL-1RA, CSF2, Chitotriosidate, and IP10 (CXCL10). In some embodiments, the biomarker panel comprises six or more of NFL, sCSF1R, sTREM2, SPP1, IL-1RA, CSF2, Chitotriosidate, and IP10 (CXCL10). In some embodiments, the biomarker panel comprises seven or more of NFL, sCSF1R, sTREM2, SPP1, IL-IRA, CSF2, Chitotriosidate, and IP10 (CXCL10). In some embodiments, the biomarker panel comprises NFL, sCSF1R, sTREM2, SPP1, IL-IRA, CSF2, Chitotriosidate, and IP10 (CXCL10).

In some embodiments, the biomarker panel comprises SPP1, IL-IRA, CSF2, Chitotriosidate, and IP10 (CXCL10). In some embodiments, the biomarker panel comprises IL-IRA, CSF2, Chitotriosidate, and IP10 (CXCL10). In some embodiments, the biomarker panel comprises SPP1, CSF2, Chitotriosidate, and IP10 (CXCL10). In some embodiments, the biomarker panel comprises SPP1, IL-1RA, Chitotriosidate, and IP10 (CXCL10). In some embodiments, the biomarker panel comprises SPP1, IL-1RA, CSF2, and IP10 (CXCL10). In some embodiments, the biomarker panel comprises SPP1, IL-1RA, CSF2, and Chitotriosidate. In some embodiments, the biomarker panel is a panel disclosed in this paragraph, further comprising NFL. In some embodiments, the biomarker panel is a panel disclosed in this paragraph, further comprising sCSF1R. In some embodiments, the biomarker panel is a panel disclosed in this paragraph, further comprising sTREM2. In some embodiments, the biomarker panel is a panel disclosed in this paragraph, further comprising a biomarker of Table A. In some embodiments, the biomarker panel is a panel disclosed in this paragraph, further comprising a biomarker of Table A*. In some embodiments, the biomarker panel is a panel disclosed in this paragraph, further comprising a biomarker of Table A**. In some embodiments, the biomarker panel is a panel disclosed in this paragraph, further comprising a biomarker of Table B.

In some embodiments, a multi-gene signature score, such as an interferon signature score, can be used as one “biomarker” in the same grouping as other individual gene biomarkers, to calculate a more predictive gene signature score.

In some embodiments of the invention, the measuring step comprises isolating RNA from the biological sample from the patient and incubating the sample with a set of probes that are designed to specifically hybridize to gene target regions of the RNA. In some embodiments, the first biological sample from the patient and/or second biological sample from the patient are assayed in vitro or ex vivo.

In some embodiments of the invention, the measuring step comprises isolating one or more proteins or variants thereof, encoded by a gene disclosed herein, from the biological sample from the patient, and measuring the concentration of said protein in the sample. In some embodiments, the first biological sample from the patient and/or second biological sample from the patient are assayed in vitro or ex vivo.

In some embodiments, the biological sample is a blood sample. In some embodiments, the biological sample is a serum sample. In some embodiments, the biological sample is a cerebrospinal fluid (CSF) sample. In some embodiments, the biological sample is a biopsied tissue sample. In some embodiments, the biological sample is a white matter brain tissue sample.

### 7.5. Binding Targets

The present invention is directed to the use of therapeutic molecules that specifically bind to TREM2, particularly human TREM2. TREM2 is a member of the Ig superfamily of receptors that is expressed on cells of myeloid lineage, including macrophages, dendritic cells, and microglia (Schmid et al., Journal of Neurochemistry, Vol. 83: 1309-1320, 2002; Colonna, Nature Reviews Immunology, Vol. 3: 445-453, 2003; Kiialainen et al., Neurobiology of Disease, 2005, 18: 314-322). TREM2 is an immune receptor that binds many endogenous substrates, including ApoE, LPS, exposed phospholipids, phosphatidylserine, and amyloid beta, and signals through a short intracellular domain that complexes with the adaptor protein DAP12, the cytoplasmic domain of which comprises an ITAM motif (Bouchon et al., The Journal of Experimental Medicine, 2001, 194: 1111-1122). Upon activation of TREM2, tyrosine residues within the ITAM motif in DAP12 are phosphorylated by the Src family of kinases, providing docking sites for the tyrosine kinase ζ-chain-associated protein 70 (ZAP70) and spleen tyrosine kinase (Syk) via their SH2 domains (Colonna, Nature Reviews Immunology, 2003, 3:445-453; Ulrich and Holtzman, ACS Chem. Neurosci., 2016, 7:420-427). The ZAP70 and Syk kinases induce activation of several downstream signaling cascades, including phosphatidylinositol 3-kinase (PI3K), protein kinase C (PKC), extracellular regulated kinase (ERK), and elevation of intracellular calcium (Colonna, Nature Reviews Immunology, 2003, 3:445-453; Ulrich and Holtzman, ACS Chem. Neurosci., 2016, 7:420-427).

TREM2 has been implicated in several myeloid cell processes, including phagocytosis, proliferation, survival, and regulation of inflammatory cytokine production (Ulrich and Holtzman, ACS Chem. Neurosci., 2016, 7: 420-427). In the last few years, TREM2 has been linked to several diseases. For instance, mutations in both TREM2 and DAP12 have been linked to the autosomal recessive disorder Nasu-Hakola Disease, which is characterized by bone cysts, muscle wasting, and demyelination phenotypes (Guerreiro et al., New England Journal of Medicine, 2013, 368: 117-127). More recently, variants in the TREM2 gene have been linked to increased risk for Alzheimer's disease (AD) and other forms of dementia including frontotemporal dementia and amyotrophic lateral sclerosis (Jonsson et al., New England Journal of Medicine, 2013, 368:107-116; Guerreiro et al., JAMA Neurology, 2013, 70:78-84; Jay et al., Journal of Experimental Medicine, 2015, 212:287-295; Cady et al, JAMA Neurol. 2014 April; 71(4):449-53). In particular, the R47H variant has been identified in genome-wide studies as being associated with increased risk for late-onset AD with an overall adjusted odds ratio (for populations of all ages) of 2.3, second only to the strong genetic association of ApoE to Alzheimer's. The R47H mutation resides on the extracellular Ig V-set domain of the TREM2 protein and has been shown to impact lipid binding and uptake of apoptotic cells and Abeta (Wang et al., Cell, 2015, 160: 1061-1071; Yeh et al., Neuron, 2016, 91: 328-340), suggestive of a loss-of-function linked to disease. Further, postmortem comparison of AD patients' brains with and without the R47H mutation are supportive of a novel loss-of-microglial barrier function for the carriers of the mutation, with the R47H carrier microglia putatively demonstrating a reduced ability to compact plaques and limit their spread (Yuan et al., Neuron, 2016, 90: 724-739). Impairment in microgliosis has been reported in animal models of prion disease, multiple sclerosis, and stroke, suggesting that TREM2 may play an important role in supporting microgliosis in response to pathology or damage in the central nervous system (Ulrich and Holtzman, ACS Chem. Neurosci., 2016, 7: 420-427).

In humans, the TREM2 gene is located within a TREM gene cluster at chromosome 6p21.1. The TREM gene cluster encodes four TREM proteins (TREM1, TREM2, TREM4, and TREM5) as well as two TREM-like proteins (TLT-1 and TLT-2). The TREM2 gene encodes a 230 amino acid protein consisting of an extracellular domain, a transmembrane region, and a short cytoplasmic tail (Paradowska-Gorycka et al., Human Immunology, Vol. 74: 730-737, 2013). The extracellular domain contains a single type V Ig-super family domain, with three potential N-glycosylation sites. The 230 amino acid wild-type hTREM2 amino acid sequence (NCBI Reference Sequence: NP_061838.1) is provided below as SEQ ID NO:1.

```
(SEQ ID NO: 1)
    1 MEPLRLLILLFVTELSGAHNTTVFQGVAGQSLQVSCPYDSMKHWGRRKAWCRQLGEKGPC
  61 QRVVSTHNLWLLSFLRRWNGSTAITDDTLGGTLTITLRNLQPHDAGLYQCQSLHGSEADT
121 LRKVLVEVLADPLDHRDAGDLWFPGESESFEDAHVEHSISRSLLEGEIPFPPTSILLLLA
181 CIFLIKILAASALWAAAWHGQKPGTHPPSELDCGHDPGYQLQTLPGLRDT
```

Amino acids 1 to 18 of the wild-type human TREM2 protein (SEQ ID NO: 1) is a signal peptide, which is generally removed from the mature protein. The mature human TREM2 protein comprises an extracellular domain at amino acids 19-174 of SEQ ID NO:1, a transmembrane domain at amino acids 175-195 of SEQ ID NO:1, and a cytoplasmic domain at amino acids 196-230 of SEQ ID NO:1. The amino acid sequence of the extracellular domain (including the signal peptide) of human TREM2 is provided below as SEQ ID NO:2.

```
(SEQ ID NO: 2)
    1 MEPLRLLILLFVTELSGAHNTTVFQGVAGQSLQVSCPYDSMKHWGRRKAWCRQLGEKGPC
  61 QRWSTHNLWLLSFLRRWNGSTAITDDTLGGTLTITLRNLQPHDAGLYQCQSLHGSEADT
121 LRKVLVEVLADPLDHRDAGDLWFPGESESFEDAHVEHSISRSLLEGEIPFPPTS
```

The term “human triggering receptor expressed on myeloid cells-2” or “human TREM2” can refer to a polypeptide of SEQ ID NO: 1, a polypeptide of SEQ ID NO:2, polypeptides of SEQ ID NO:1 or SEQ ID NO:2 minus the signal peptide (amino acids 1-18), allelic variants of human TREM2, or splice variants of human TREM2. In some embodiments, the term “human TREM2” includes naturally occurring variants of TREM2, such as mutations R47H, Q33X (X is a stop codon), Y38C, T66M, D87N, H157Y, R98W, and S116C.

Because the cytoplasmic domain of TREM2 lacks signaling capability, it must interact with other proteins to transduce TREM2-activating signals. One such protein is DNAX-activating protein of 12 kDa (DAP12). DAP12 is also known as killer cell activating receptor-associated protein (KARAP) and tyrosine kinases binding protein (TYROBP). DAP12 is a type I transmembrane adaptor protein that comprises an ITAM motif in its cytoplasmic domain. The ITAM motif mediates signal propagation by activation of the ZAP70 and Syk tyrosine kinases, which in turn activate several downstream signaling cascades, including PI3K, PKC, ERK, and elevation of intracellular calcium (Colonna, Nature Reviews Immunology, Vol. 3: 445-453, 2003; Ulrich and Holtzman, ACS Che Neurosci., Vol. 7: 420-427, 2016). DAP12 and TREM2 associate through their transmembrane domains; a charged lysine residue within the transmembrane domain of TREM2 interacts with a charged aspartic acid residue within the transmembrane domain of DAP12.

Human DAP12 is encoded by the TYROBP gene located on chromosome 19q13.1. The human protein is 113 amino acids in length and comprises a leader sequence (amino acids 1-27 of SEQ ID NO:3), a short extracellular domain (amino acids 28-41 of SEQ ID NO:3), a transmembrane domain (amino acids 42-65 of SEQ ID NO:3), and a cytoplasmic domain (amino acids 66-113 of SEQ ID NO:3) (Paradowska-Gorycka et al., Human Immunology, Vol. 74: 730-737, 2013). DAP12 forms a homodimer through two cysteine residues in the short extracellular domain. The wild-type human DAP12 amino acid sequence (NCBI Reference Sequence: NP_003323.1) is provided below as SEQ ID NO:3.

(SEQ ID NO: 3)
1 MGGLEPCSRLLLLPLLLAVSGLRPVQAQAQSDCSCSTVSPGVLAGIVMGDLVLTVLIALA
61 VYFLGRLVPRGRGAAEAATRKQRITETESPYQELQGQRSDVYSDLNTQRPYY

The term “human DAP12” can refer to a polypeptide of SEQ ID NO:3, a polypeptide of SEQ ID NO:3 minus the leader peptide (amino acids 1-27), allelic variants of human DAP12, or splice variants of human DAP12.

### 7.6. Treatment Methods of the Invention

In one aspect, the present invention provides a method of treating a disease or disorder caused by and/or associated with a TREM2 dysfunction in a human patient, the method comprising administering to the patient a molecule that increases activity of TREM2. In some embodiments, the molecule that increases activity of TREM2 is an agonist of TREM2. In some embodiments, the agonist of TREM2 is an anti-hTREM2 antibody, or an antigen binding-fragment thereof. In some embodiments, the agonist of TREM2 is a small molecule. In some embodiments, the molecule that increases activity of TREM2 is a molecule that prevents the degradation of TREM2. In some embodiments, the molecule that increases activity of TREM2 is an anti-hTREM2 antibody, or an antigen binding-fragment thereof. In some embodiments, the molecule that increases activity of TREM2 is a small molecule.

In some embodiments, administration of the agonist of TREM2 activates DAP12 signaling pathways in the patient, resulting in an increase in microglia proliferation, microglia survival, and/or microglia phagocytosis. In some embodiments, administration of the agonist of TREM2 results in a slowing of disease progression.

In some embodiments, the agonist of TREM2 activates TREM2/DAP12 signaling in myeloid cells, including monocytes, dendritic cells, microglial cells, and/or macrophages. In some embodiments, an agonist of TREM2 activates, induces, promotes, stimulates, or otherwise increases one or more TREM2 activities. TREM2 activities that are activated or increased by the agonist, include but are not limited to: TREM2 binding to DAP12; DAP12 binding to TREM2; TREM2 phosphorylation, DAP12 phosphorylation; PI3K activation; AKT activation; increased levels of soluble TREM2 (sTREM2); decreased levels of soluble TREM2 (sTREM2); increased levels of soluble CSF1R (sCSF1R); increased expression of CSF1R; increased expression of one or more anti-inflammatory mediators (e.g., cytokines) selected from the group consisting of IL-12p70, IL-6, and IL-10; reduced expression of one or more pro-inflammatory mediators selected from the group consisting of IFN-a4, IFN-b, IL-6, IL-12 p70, IL-1β, TNF, TNF-α, IL-10, IL-8, CRP, TGF-beta members of the chemokine protein families, IL-20 family members, IL-33, LIF, IFN-gamma, OSM, CNTF, TGF-beta, GM-CSF, IL-11, IL-12, IL-17, IL-18, and CRP; increased expression of one or more chemokines selected from the group consisting of CCL2, CCL4, CXCL10, CXCL2, and CST7; reduced expression of TNF-α, IL-6, or both; extracellular signal-regulated kinase (ERK) phosphorylation; increased expression of C—C chemokine receptor 7 (CCR7); induction of microglial cell chemotaxis toward CCL19 and CCL21 expressing cells; an increase, normalization, or both of the ability of bone marrow-derived dendritic cells to induce antigen-specific T-cell proliferation; induction of osteoclast production, increased rate of osteoclastogenesis, or both; increasing the survival and/or function of one or more of dendritic cells, macrophages, microglial cells, M1 macrophages and/ormicroglial cells, activated M1 macrophages and/ormicroglial cells, M2 macrophages and/or microglial cells, monocytes, osteoclasts, Langerhans cells of skin, and Kupffer cells; induction of one or more types of clearance selected from the group consisting of apoptotic neuron clearance, nerve tissue debris clearance, non-nerve tissue debris clearance, bacteria or other foreign body clearance, disease-causing protein clearance, disease-causing peptide clearance, and disease-causing nucleic acid clearance; induction of phagocytosis of one or more of apoptotic neurons, nerve tissue debris, non-nerve tissue debris, bacteria, other foreign bodies, disease-causing proteins, disease-causing peptides, or disease-causing nucleic acids; normalization of disrupted TREM2/DAP12-dependent gene expression; recruitment of Syk, ZAP70, or both to the TREM2/DAP12 complex; Syk phosphorylation; increased expression of CD83 and/or CD86 on dendritic cells, macrophages, monocytes, and/or microglia; reduced secretion of one or more inflammatory cytokines selected from the group consisting of TNF-α, IL-10, IL-6, MCP-1, IFN-α4, IFN-b, IL-10, IL-8, CRP, TGF-beta members of the chemokine protein families, IL-20 family members, IL-33, LIF, IFN-gamma, OSM, CNTF, TGF-beta, GM-CSF, IL-11, IL-12, IL-17, IL-18, and CRP; reduced expression of one or more inflammatory receptors; increasing phagocytosis by macrophages, dendritic cells, monocytes, and/or microglia under conditions of reduced levels of MCSF; decreasing phagocytosis by macrophages, dendritic cells, monocytes, and/or microglia in the presence of normal levels of MCSF; increasing activity of one or more TREM2-dependent genes; or any combination thereof. In some embodiments, an agonist of TREM2 increases one or more TREM2 signaling related activities selected from microglial function, innate immunity, growth factor signaling, and cell cycle processes.

In another aspect, the invention provides a TREM2 agonist for the manufacture of a medicament for the treatment of a condition or disorder associated with microglial dysfunction. In another aspect, the invention provides a TREM2 agonist for the manufacture of a medicament for the treatment of a disease or disorder caused by and/or associated with a TREM2 dysfunction. In yet another aspect, the invention provides a TREM2 agonist for the treatment of a condition or disorder associated with microglial dysfunction. In yet another aspect, the invention provides a TREM2 agonist for the treatment of a disease or disorder caused by and/or associated with a TREM2 dysfunction.

### 7.7. Diseases and Disorders

Certain aspects of the present invention provide a TREM2 agonist for use in treating, preventing, or ameliorating the risk of developing conditions associated with microglial dysfunction in a patient in need thereof. In some embodiments, the microglial dysfunction is caused by a TREM2 deficiency. In some embodiments, the microglial dysfunction is caused by dysfunction of colony stimulating factor 1 receptor (CSF1R). In some embodiments, the microglial dysfunction is caused by dysfunction of ATP-binding cassette transporter 1 (ABCD1). In particular embodiments, the use comprises administering to the patient an effective amount of a TREM2 agonist.

Conditions or disorders associated with TREM2 deficiency or loss of TREM2 function that may be prevented, treated, or ameliorated according to the methods of the invention include, but are not limited to, Nasu-Hakola disease, Alzheimer's disease, frontotemporal dementia, multiple sclerosis, Guillain-Barre syndrome, amyotrophic lateral sclerosis, Parkinson's disease, traumatic brain injury, spinal cord injury, systemic lupus erythematosus, rheumatoid arthritis, prion disease, stroke, osteoporosis, osteopetrosis, and osteosclerosis. In certain embodiments, the condition or disorder to be prevented, treated, or ameliorated according to the methods of the invention is Alzheimer's disease, Nasu-Hakola disease, frontotemporal dementia, multiple sclerosis, prion disease, or stroke.

In some embodiments, conditions or disorders that can be prevented, treated, or ameliorated according to the methods of the invention are ones that are associated with dysfunction of CSF1R. Conditions or disorders associated with dysfunction of CSF1R that may be prevented, treated, or ameliorated according to the methods of the invention include, but are not limited to, adult-onset leukoencephalopathy with axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS). In some embodiments, conditions or disorders associated with dysfunction of CSF1R also include Nasu-Hakola disease, Alzheimer's disease, frontotemporal dementia, multiple sclerosis, Guillain-Barre syndrome, amyotrophic lateral sclerosis (ALS), Parkinson's disease, traumatic brain injury, spinal cord injury, systemic lupus erythematosus, rheumatoid arthritis, prion disease, stroke, osteoporosis, osteopetrosis, osteosclerosis, skeletal dysplasia, dysosteoplasia, Pyle disease, cerebral autosomal dominant arteriopathy with subcortical infarcts and leukoencephalopathy, cerebral autosomal recessive arteriopathy with subcortical infarcts and leukoencephalopathy, cerebroretinal vasculopathy, or metachromatic leukodystrophy, wherein any of the aforementioned diseases or disorders are present in a patient exhibiting CSF1R dysfunction, or having a mutation in a gene affecting the function of CSF1R.

In some embodiments, conditions or disorders that can be prevented, treated, or ameliorated according to the methods of the invention are ones that are associated with dysfunction of ABCD1. Conditions or disorders associated with dysfunction of ABCD1 that may be prevented, treated, or ameliorated according to the methods of the invention include, but are not limited to, X-linked adrenoleukodystrophy (x-ALD), childhood cerebral adrenoleukodystrophy (cALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX). In some embodiments, conditions or disorders associated with dysfunction of ABCD1 also include Nasu-Hakola disease, Alzheimer's disease, frontotemporal dementia, multiple sclerosis, Guillain-Barre syndrome, amyotrophic lateral sclerosis (ALS), Parkinson's disease, traumatic brain injury, spinal cord injury, systemic lupus erythematosus, rheumatoid arthritis, prion disease, stroke, osteoporosis, osteopetrosis, osteosclerosis, skeletal dysplasia, dysosteoplasia, Pyle disease, cerebral autosomal dominant arteriopathy with subcortical infarcts and leukoencephalopathy, cerebral autosomal recessive arteriopathy with subcortical infarcts and leukoencephalopathy, cerebroretinal vasculopathy, or metachromatic leukodystrophy, wherein any of the aforementioned diseases or disorders are present in a patient exhibiting ABCD1 dysfunction, or having a mutation in a gene affecting the function of ABCD1.

In some embodiments, the condition to be treated, prevented, or ameliorated is Alzheimer's disease. In some embodiments, the patient to be administered a TREM2 agonist antigen binding protein is a patient at risk of developing Alzheimer's disease. The patient in need of treatment may be determined to have one or more genotypes associated with an increased risk of developing a disease or condition that can be treated according to the methods of the invention. For instance, in some embodiments, the patient has a genotype associated with an increased risk of developing Alzheimer's disease, such as the genotypes described herein. In further embodiments, the patient may be determined to carry an allele encoding a TREM2 variant associated with an increased risk of developing Alzheimer's disease. For instance, in one embodiment, the patient has been determined to have at least one allele containing the rs75932628-T mutation in the TREM2 gene, e.g., the patient has a genotype of CT at rs75932628. In related embodiments, the patient having or at risk of developing Alzheimer's disease is a patient who has been determined to carry a TREM2 variant allele that encodes a histidine in place of arginine at position 47 in SEQ ID NO:1 (R47H TREM2 variant). In other embodiments, the patient has been determined to have at least one allele containing the rsl43332484-T mutation in the TREM2 gene, e.g., the patient has a genotype of CT at rsl43332484. In related embodiments, the patient having or at risk of developing Alzheimer's disease is a patient who has been determined to carry a TREM2 variant allele that encodes a histidine in place of arginine at position 62 in SEQ ID NO:1 (R62H TREM2 variant). In some embodiments, a patient at risk of developing Alzheimer's disease has been determined to have at least one allele containing the rs6910730-G mutation in the TREM1 gene, at least one allele containing the rs7759295-C mutation upstream of the TREM2 gene, and/or at least one E4 allele of the APOE gene.

In another embodiment, the present invention provides a method for preventing, treating, or ameliorating frontotemporal dementia or Nasu-Hakola disease in a patient in need thereof comprising administering to the patient an effective amount of a TREM2 agonist antigen binding protein described herein. In some embodiments, the patient to be administered a TREM2 agonist antigen binding protein is a patient at risk of developing frontotemporal dementia or Nasu-Hakola disease. For example, in one such embodiment, the patient has been determined to have at least one allele containing the rs104894002-A mutation in the TREM2 gene, e.g., the patient has a genotype of GA or AA at rs 104894002. In related embodiments, the patient at risk of developing frontotemporal dementia or Nasu-Hakola disease is a patient who has been determined to carry a TREM2 variant allele that encodes a truncated TREM2 protein as a result of the substitution of a stop codon in place of glutamine at position 33 in SEQ ID NO: 1. In another embodiment, the patient has been determined to have at least one allele containing the rs201258663-A mutation in the TREM2 gene, e.g., the patient has a genotype of GA or AA at rs201258663. In related embodiments, the patient at risk of developing frontotemporal dementia or Nasu-Hakola disease is a patient who has been determined to have any TREM2 variant allele that encodes a methionine in place of threonine at position 66 in SEQ ID NO: 1. In some embodiments, the patient at risk of developing frontotemporal dementia or Nasu-Hakola disease is a patient who has been determined to carry a TREM2 variant allele that encodes a cysteine in place of tyrosine at position 38 in SEQ ID NO:1.

In yet another embodiment, the present invention provides a method for preventing, treating, or ameliorating multiple sclerosis in a patient in need thereof, comprising administering to the patient an effective amount of a TREM2 agonist described herein. In some embodiments, the patient to be administered a TREM2 agonist is a patient at risk of developing multiple sclerosis.

The present invention also includes methods of increasing survival or proliferation of myeloid cells, such as macrophages, microglia, and dendritic cells, in a patient in need thereof. In some embodiments, TREM2 agonist described herein can be used to activate TREM2/DAP12 signaling in myeloid cells, thereby modulating the biological activity of these cells. Such biological activities include cytokine release, phagocytosis, and microgliosis.

The TREM2 agonists described herein can be used in the manufacture of a pharmaceutical composition or medicament for the treatment or prevention of conditions associated with TREM2 deficiency or loss of TREM2 biological activity as described herein, including, inter alia, Alzheimer's disease, Nasu-Hakola disease, frontotemporal dementia, multiple sclerosis, prion disease, or stroke. Thus, the present invention also provides a pharmaceutical composition comprising a TREM2 agonist antigen binding protein described herein and a pharmaceutically acceptable excipient.

In one embodiment, the present invention provides a method for preventing, treating, or ameliorating Alzheimer's disease in a patient in need thereof, comprising administering to the patient an effective amount of a TREM2 agonist antigen binding protein described herein. In certain embodiments, the TREM2 agonist administered to the patient is an anti-hTREM2 monoclonal antibody, such as the antibodies whose CDR sequences, variable region sequences, and heavy and light chain sequences are set forth in Tables 1A-1B and 2.

### 7.8. Target Agonists

The present invention provides for methods of treating, preventing, or ameliorating the risk of developing conditions associated with TREM2 deficiency in a patient in need thereof, the method comprising administering to the patient an effective amount of a molecule that specifically binds to hTREM2, which increases the activity of hTREM2. In some embodiments, the molecule is an agonist of TREM2. In some embodiments, the agonist of TREM2 is a small molecule. In some embodiments, the agonist of TREM2 is an antibody, or antigen-binding fragment thereof.

The TREM2 agonist specifically bind to human TREM2 (SEQ ID NO: 1) or an extracellular domain (ECD) of human TREM2 (e.g., ECD set forth in SEQ ID NO:2), for example with an equilibrium dissociation constant (K_{D}) less than 50 nM, less than 25 nM, less than 10 nM, or less than 5 nM.

### 7.8.1. Anti-hTREM2 Antibodies

### Antibodies

In one aspect, the invention relates to administration of anti-hTREM2 antibodies, or antigen-binding fragments thereof. While certain embodiments are provided for in the context of intact antibodies, it is contemplated that molecules derived from the antigen-binding fragment of said antibodies may maintain binding specificity and can also be used in the present invention.

In some embodiments, the anti-hTREM2 antibodies are agonists of hTREM2. In some embodiments, the anti-hTREM2 antibodies do not cross-react with other TREM proteins, such as human TREM1 (hTREM1). In some embodiments, the anti-hTREM2 antibodies do not bind to hTREM1, or an isoform or truncation thereof. The amino acid sequence of precursor hTREM1 isoform 1 (NCBI Reference Sequence: NP 061113.1) is provided below as SEQ ID NO:4.

```
(SEQ ID NO: 4)
    1 MRKTRLWGLLWMLFVSELRAATKLTEEKYELKEGQTLDVKCDYTLEKFASSQKAWQIIRD
  61 GEMPKTLACTERPSKNSHPVQVGRIILEDYHDHGLLRVRMVNLQVEDSGLYQCVIYQPPK
121 EPHMLFDRIRLVVTKGFSGTPGSNENSTQNVYKIPPTTTKALCPLYTSPRTVTQAPPKST
181 ADVSTPDSEINLTNVTDIIRVPVFNIVILLAGGFLSKSLVFSVLFAVTLRSFVP
```

In some embodiments, the anti-hTREM2 antibodies specifically bind to human TREM2 hTREM2 residues 19-174. In some embodiments, the anti-hTREM2 antibodies specifically bind to IgV region of hTREM2, for example human TREM2 residues 19-140.

In certain embodiments, anti-hTREM2 antibodies of the present disclosure bind to one or more amino acids within amino acid residues 29-112 of hTREM 2 (SEQ ID NO: 1), or within amino acid residues on a TREM2 protein corresponding to amino acid residues 29-112 of SEQ ID NO: 1. In some embodiments, anti-hTREM2 antibodies of the present disclosure bind to one or more amino acids within amino acid residues 29-41 of hTREM 2 (SEQ ID NO: 1), or within amino acid residues on a TREM2 protein corresponding to amino acid residues 29-41 of SEQ ID NO: 1. In some embodiments, anti-hTREM2 antibodies of the present disclosure bind to one or more amino acids within amino acid residues 47-69 of hTREM 2 (SEQ ID NO: 1), or within amino acid residues on a TREM2 protein corresponding to amino acid residues 47-69 of SEQ ID NO: 1. In some embodiments, anti-hTREM2 antibodies of the present disclosure bind to one or more amino acids within amino acid residues 76-86 of hTREM 2 (SEQ ID NO: 1), or within amino acid residues on a TREM2 protein corresponding to amino acid residues 76-86 of SEQ ID NO: 1. In some embodiments, anti-hTREM2 antibodies of the present disclosure bind to one or more amino acids within amino acid residues 91-100 of hTREM2 (SEQ ID NO: 1), or within amino acid residues on a TREM2 protein corresponding to amino acid residues 91-100 of SEQ ID NO: 1. In some embodiments, anti-hTREM2 antibodies of the present disclosure bind to one or more amino acids within amino acid residues 99-115 of hTREM 2 (SEQ ID NO: 1), or within amino acid residues on a TREM2 protein corresponding to amino acid residues 99-115 of SEQ ID NO: 1. In some embodiments, anti-hTREM2 antibodies of the present disclosure bind to one or more amino acids within amino acid residues 104-112 of hTREM 2 (SEQ ID NO: 1), or within amino acid residues on a TREM2 protein corresponding to amino acid residues 104-112 of SEQ ID NO: 1. In some embodiments, anti-hTREM2 antibodies of the present disclosure bind to one or more amino acids within amino acid residues 114-118 of hTREM 2 (SEQ ID NO: 1), or within amino acid residues on a TREM2 protein corresponding to amino acid residues 114-118 of SEQ ID NO: 1. In some embodiments, anti-hTREM2 antibodies of the present disclosure bind to one or more amino acids within amino acid residues 130-171 of hTREM 2 (SEQ ID NO: 1), or within amino acid residues on a TREM2 protein corresponding to amino acid residues 130-171 of SEQ ID NO: 1. In some embodiments, anti-hTREM2 antibodies of the present disclosure bind to one or more amino acids within amino acid residues 139-153 of hTREM 2 (SEQ ID NO: 1), or within amino acid residues on a TREM2 protein corresponding to amino acid residues 139-153 of SEQ ID NO: 1. In some embodiments, anti-hTREM2 antibodies of the present disclosure bind to one or more amino acids within amino acid residues 139-146 of hTREM 2 (SEQ ID NO: 1), or within amino acid residues on a TREM2 protein corresponding to amino acid residues 139-146 of SEQ ID NO: 1. In some embodiments, anti-hTREM2 antibodies of the present disclosure bind to one or more amino acids within amino acid residues 130-144 of hTREM 2 (SEQ ID NO: 1), or within amino acid residues on a TREM2 protein corresponding to amino acid residues 130-144 of SEQ ID NO: 1. In some embodiments, anti-hTREM2 antibodies of the present disclosure bind to one or more amino acids within amino acid residues 158-171 of hTREM 2 (SEQ ID NO: 1), or within amino acid residues on a TREM2 protein corresponding to amino acid residues 158-171 of SEQ ID NO:1.

In some embodiments, anti-hTREM2 antibodies of the present disclosure bind to one or more amino acids within amino acid residues 43-50 of hTREM 2 (SEQ ID NO: 1), or within amino acid residues on a TREM2 protein corresponding to amino acid residues 43-50 of SEQ ID NO: 1. In some embodiments, anti-hTREM2 antibodies of the present disclosure bind to one or more amino acids within amino acid residues 49-57 of hTREM 2 (SEQ ID NO: 1), or within amino acid residues on a TREM2 protein corresponding to amino acid residues 49-57 of SEQ ID NO: 1. In some embodiments, anti-hTREM2 antibodies of the present disclosure bind to one or more amino acids within amino acid residues 139-146 of hTREM 2 (SEQ ID NO: 1), or within amino acid residues on a TREM2 protein corresponding to amino acid residues 139-146 of SEQ ID NO: 1. In some embodiments, anti-hTREM2 antibodies of the present disclosure bind to one or more amino acids within amino acid residues 140-153 of hTREM 2 (SEQ ID NO: 1), or within amino acid residues on a TREM2 protein corresponding to amino acid residues 140-153 of SEQ ID NO: 1. In some embodiments, anti-hTREM2 antibodies specifically bind to the stalk region of human TREM2, for example amino acid residues 145-174 of human TREM2.

In some embodiments, the anti-hTREM2 antibody, or an antigen-binding fragment thereof, specifically prevents the degradation or cleavage of hTREM2.

As used herein, the term “antibody” (Ab) refers to an immunoglobulin molecule that specifically binds to a particular antigen, e.g., hTREM2. In some embodiments, an anti-hTREM2 antibody is suitable for administration to humans.

In some embodiments, the anti-hTREM2 antibody is a polyclonal antibody. In some embodiments, the anti-hTREM2 antibody is a monoclonal antibody. In some embodiments, the anti-hTREM2 antibody is a chimeric antibody. In some embodiments, the anti-hTREM2 antibody is a humanized antibody. In some embodiments, the anti-hTREM2 antibody is a human antibody, particularly a fully human antibody. In some embodiments, the anti-hTREM2 antibody is a bispecific or other multivalent antibody. In some embodiments, the antibody is a single chain antibody.

In some embodiments, the antibodies comprise all or a portion of a constant region of an antibody. In some embodiments, the constant region is a selected from an isotype selected from: IgA (e.g., IgA₁ or IgA₂), IgD, IgE, IgG (e.g., IgG₁, IgG₂, IgG₃ or IgG₄), or IgM. In specific embodiments, the anti-hTREM2 antibodies described herein comprise an IgG₁. In some embodiments, the constant region of the IgG₁ comprises a substitution selected from R292C, N297G, V302C, D356E, or L358M (according to EU numbering). In other embodiments, the anti-hTREM2 antibodies comprise an IgG2. In other embodiments, the anti-hTREM2 antibodies comprise an IgG₄. As used herein, the “constant region” of an antibody includes the natural constant region, or any allotypes or natural variants thereof.

The light constant region of an anti-hTREM2 antibody may comprise a lambda (Q) light region or a kappa (x) light region. The a light region can be any one of the known subtypes, e.g., λ₁, λ₂, λ₃, or λ₄.

The term “monoclonal antibody” as used herein is not limited to antibodies produced through hybridoma technology. A monoclonal antibody is derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

The term “chimeric” antibody as used herein refers to an antibody having variable sequences derived from an immunoglobulin of one species, such as a rat or a mouse antibody, and an immunoglobulin constant region of another species, such as a human immunoglobulin template. Other examples of a chimeric antibody include a human derived immunoglobulin variable region with a murine immunoglobulin constant region.

“Humanized” forms of non-human (e.g., murine) antibodies comprise substantially all of the CDR regions and variable regions of a non-human immunoglobulin and all or substantially all of the FR regions of a human immunoglobulin sequence. The humanized antibody may also comprise at least a portion of an immunoglobulin constant region (Fc).

“Human antibodies” include antibodies having the amino acid sequence of a human immunoglobulin. Human antibodies can be from animals that are transgenic for one or more human immunoglobulins. For example, transgenic animals may lack endogenous production of one or more immunoglobulins, such as the Xenomouse®, and be engineered to produce antibodies with fully human protein sequences upon immunization. Human antibodies can also be made by a variety of methods known in the art, including isolation from human immunoglobulin libraries, or phage display methods using antibody libraries derived from human immunoglobulin sequences.

Anti-hTREM2 antibodies of the disclosure include full-length (intact) antibody molecules, or portions thereof. The anti-hTREM2 antibodies may be antibodies whose sequences have been modified to alter at least one constant region-mediated biological effector function (e.g., improved or reduced binding to one or more of the Fc receptors (FcγR), such as FcγRI, FcγRIIA, FcγRIIB, FcγRIIIA, and/or FcγRIIIB).

Anti-hTREM2 antibodies with high affinity for hTREM2 may be desirable for therapeutic and diagnostic uses. Accordingly, the present disclosure contemplates antibodies having a high binding affinity to hTREM2. In specific embodiments, the anti-hTREM2 antibodies binds to hTREM2 with an affinity of at least about 100 nM, but may exhibit higher affinity, for example, at least about 90 nM, 80 nM, 70 nM, 60 nM, 50 nM, 40 nM, 30 nM, 25 nM, 20 nM, 15 nM, 10 nM, 7 nM, 6 nM, 5 nM, 4 nM, 3 nM, 2 nM, 1 nM, 0.1 nM, 0.01 nM, or even higher. In some embodiments, the antibodies bind hTREM2 with an affinity in the range of about 1 pM to about 10 nM, of about 100 pM to about 10 nM, about 100 pM to about 1 nM, or an affinity ranging between any of the foregoing values.

Affinity of anti-hTREM2 antibodies for hTREM2 can be determined using techniques well known in the art or described herein, such as for example, but not by way of limitation, ELISA, isothermal titration calorimetry, surface plasmon resonance, biolayer inferotometry, filter binding, or fluorescent polarization.

Anti-hTREM2 antibodies of the disclosure comprise complementarity determining regions (CDRs) in both the light chain and the heavy chain variable domains. Anti-hTREM2 antibodies comprise a light chain variable region comprising complementarity determining regions CDRL1, CDRL2, and CDRL3 and a heavy chain variable region comprising complementarity determining regions CDRH1, CDRH2, and CDRH3 described herein.

In some embodiments, the TREM2 agonist antigen binding protein comprises a CDRL1 or a variant thereof having one, two, three or four amino acid substitutions; a CDRL2, or a variant thereof having one, two, three or four amino acid substitutions; a CDRL3, or a variant thereof having one, two, three or four amino acid substitutions; a CDRH1, or a variant thereof having one, two, three or four amino acid substitutions; a CDRH2, or a variant thereof having one, two, three or four amino acid substitutions; and a CDRH3, or a variant thereof having one, two, three or four amino acid substitutions, where the amino acid sequences of the CDRL1, CDRL2, CDRL3, CDRH1, CDRH2, and CDRH3 are provided in Tables 1A and 1B below, along with exemplary light chain and variable regions.

TABLE 1A
Exemplary Anti-hTREM2 Antibody Light Chain Variable Regions

| VL Amino Acid Sequence | CDRL1 | CDRL2 | CDRL3 |
|---|---|---|---|
| EIVMTQSPATLSVSPGERATLSCRASQSVSSNLA | RASQSVSSNLA | GASTRAT | LQDNNFPPT |
| WFQQKPGQAPRLLIYGASTRATGIPARFSGSGSG | SEQ ID | SEQ ID | SEQ ID |
| TEFTLTISSLQPEDFAVYYCLQDNNFPPTFGQGT | NO: 6 | NO: 7 | NO: 8 |
| KVDIK | | | |
| SEQ ID NO: 5 | | | |

TABLE 1B
Exemplary Anti-hTREM2 Antibody Heavy Chain Variable Regions

| VH Amino Acid Sequence | CDRH1 | CDRH2 | CDRH3 |
|---|---|---|---|
| EVQLVQSGAEVKKPGESLKISCKGSGYSFTS | SYWIG | IIYPGDADA | RRQGIF |
| YWIGWVRQMPGKGLEWMGIIYPGDADARYSP | SEQ ID | RYSPSFQG | GDALDF |
| SFQGQVTISADKSISTAYLQWSSLKASDTAM | NO: 10 | SEQ ID | SEQ ID |
| YFCARRRQGIFGDALDFWGQGTLVTVSS | | NO: 11 | NO: 12 |
| SEQ ID NO: 9 | | | |

As noted above, anti-hTREM2 antibodies may comprise one or more of the CDRs presented in Table 1A (light chain CDRs; i.e., CDRLs) and Table 1B (heavy chain CDRs, i.e., CDRHs).

In some embodiments, an anti-hTREM2 antibody comprises a light chain comprising a CDRL1 having an amino acid sequence according to SEQ ID NO:6, a CDRL2 having an amino acid sequence according to SEQ ID NO:7, a CDRL3 having an amino acid sequence according to SEQ ID NO:8, or any CDRL1, CDRL2, or CDRL3 amino acid sequence that contains one or more, e.g., one, two, three, four or more amino acid substitutions (e.g., conservative amino acid substitutions), deletions or insertions of no more than five, four, three, two, or one amino acids to any of SEQ ID NOS:6-8. Such substitutions, deletions, and insertions would retain significant anti-hTREM2 binding activity. In these and other embodiments, an anti-hTREM2 antibody comprises a CDRH1 having an amino acid sequence according to SEQ ID NO: 10, a CDRH2 having an amino acid sequence according to SEQ ID NO:11, a CDRH3 having an amino acid sequence according to SEQ ID NO: 12, or any CDRH1, CDRH2, or CDRH3 having an amino acid sequence that contains one or more, e.g., one, two, three, four or more amino acid substitutions (e.g., conservative amino acid substitutions), deletions or insertions of no more than five, four, three, two, or one amino acids to any of SEQ ID NOS: 10-12. Such substitutions, deletions, and insertions would retain significant anti-hTREM2 binding activity.

In some embodiments, an anti-hTREM2 antibody comprises a light chain variable region comprising a CDRL1 having an amino acid sequence according to SEQ ID NO:6; a CDRL2 having an amino acid sequence according to SEQ ID NO:7; and a CDRL3 having an amino acid sequence according to SEQ ID NO:8; and a heavy chain variable region comprising a CDRH1 having an amino acid sequence according to SEQ ID NO: 10; a CDRH2 having an amino acid sequence according to SEQ ID NO: 11; and a CDRH3 having an amino acid sequence according to SEQ ID NO: 12.

In some embodiments, an anti-hTREM2 antibody comprises a light chain variable region having an amino acid sequence according to SEQ ID NO:5, or any amino acid sequence that contains one or more, e.g., one, two, three, four or more amino acid substitutions (e.g., conservative amino acid substitutions), deletions or insertions of no more than five, four, three, two, or one amino acids to SEQ ID NO:5. Such substitutions, deletions, and insertions would retain significant anti-hTREM2 binding activity. In some embodiments, an anti-hTREM2 antibody comprises a heavy chain variable region having an amino acid sequence according to SEQ ID NO:9, or any amino acid sequence that contains one or more, e.g., one, two, three, four or more amino acid substitutions (e.g., conservative amino acid substitutions), deletions or insertions of no more than five, four, three, two, or one amino acids to SEQ ID NO:9. Such substitutions, deletions, and insertions would retain significant anti-hTREM2 binding activity.

In specific embodiments, an anti-hTREM2 antibody comprises a light chain variable region having an amino acid sequence according to SEQ ID NO:5, and a heavy chain variable region having an amino acid sequence according to SEQ ID NO:9.

In some embodiments, an anti-hTREM2 antibody comprises a heavy chain amino acid sequence, and/or a light chain amino acid sequence selected from Table 2. Table 2 shows exemplary anti-hTREM2 antibody heavy and light chains for exemplary antibodies “Ab-1” and “Ab-2”.

TABLE 2
Exemplary Anti-hTREM2 Antibody Heavy and Light Chains

| Chain | Description | Amino Acid Sequence |
|---|---|---|
| Light | Ab-1 | EIVMTQSPATLSVSPGERATLSCRASQSVSSNLAWFQQKPGQAPRLLIYGASTRATGIPAR |
| Chain | | FSGSGSGTEFTLTISSLQPEDFAVYYCLQDNNFPPTFGQGTKVDIKRTVAAPSVFIFPPSD |
| | | EQLKSGTASVVCLLNNFYPREAKVQWKVDNALQSGNSQESVTEQDSKDSTYSLSSTLTLSK |
| | | ADYEKHKVYACEVTHQGLSSPVTKSFNRGEC |
| | | (SEQ ID NO: 13) |
| | Ab-1 w/ | MDMRVPAQLLGLLLLWLRGARCEIVMTQSPATLSVSPGERATLSCRASQSVSSNLAWFQQK |
| | leader | PGQAPRLLIYGASTRATGIPARFSGSGSGTEFTLTISSLQPEDFAVYYCLQDNNFPPTFGQ |
| | sequence | GTKVDIKRTVAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| | | (SEQ ID NO: 14) |
| | Ab-2 | EIVMTQSPATLSVSPGERATLSCRASQSVSSNLAWFQQKPGQAPRLLIYGASTRATGIPAR |
| | | FSGSGSGTEFTLTISSLQPEDFAVYYCLQDNNFPPTFGQGTKVDIKRADAAPTVSIFPPSS |
| | | EQLTSGGASVVCFLNNFYPKDINVKWKIDGSERQNGVLNSWTDQDSKDSTYSMSSTLTLTK |
| | | DEYERHNSYTCEATHKTSTSPIVKSFNRNEC |
| | | (SEQ ID NO: 15) |
| Heavy | Ab-1 | EVQLVQSGAEVKKPGESLKISCKGSGYSFTSYWIGWVRQMPGKGLEWMGIIYPGDADARYS |
| Chain | | PSFQGQVTISADKSISTAYLQWSSLKASDTAMYFCARRRQGIFGDALDFWGQGTLVTVSSA |
| | | STKGPSVFPLAPSSKSTSGGTAALGCLVKDYFPEPVTVSWNSGALTSGVHTFPAVLQSSGL |
| | | YSLSSVVTVPSSSLGTQTYICNVNHKPSNTKVDKKVEPKSCDKTHTCPPCPAPELLGGPSV |
| | | FLFPPKPKDTLMISRTPEVTCVVVDVSHEDPEVKFNWYVDGVEVHNAKTKPCEEQYGSTYR |
| | | CVSVLTVLHQDWLNGKEYKCKVSNKALPAPIEKTISKAKGQPREPQVYTLPPSREEMTKNQ |
| | | VSLTCLVKGFYPSDIAVEWESNGQPENNYKTTPPVLDSDGSFFLYSKLTVDKSRWQQGNVF |
| | | SCSVMHEALHNHYTQKSLSLSPGK |
| | | (SEQ ID NO: 16) |
| | Ab-1 w/ | MDMRVPAQLLGLLLLWLRGARCEVQLVQSGAEVKKPGESLKISCKGSGYSFTSYWIGWVRQ |
| | leader | MPGKGLEWMGIIYPGDADARYSPSFQGQVTISADKSISTAYLQWSSLKASDTAMYFCARRR |
| | sequence | QGIFGDALDFWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTKVDKKVEPK |
| | | SCDKTHTCPPCPAPELLGGPSVFLFPPKPKDTLMISRTPEVTCVVVDVSHEDPEVKFNWYV |
| | | DGVEVHNAKTKPCEEQYGSTYRCVSVLTVLHQDWLNGKEYKCKVSNKALPAPIEKTISKAK |
| | | GQPREPQVYTLPPSREEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| | | (SEQ ID NO: 17) |
| | Ab-2 | EVQLVQSGAEVKKPGESLKISCKGSGYSFTSYWIGWVRQMPGKGLEWMGIIYPGDADARYS |
| | | PSFQGQVTISADKSISTAYLQWSSLKASDTAMYFCARRRQGIFGDALDFWGQGTLVTVSSA |
| | | KTTPPSVYPLAPGSAAQTNSMVTLGCLVKGYFPEPVTVTWNSGSLSSGVHTFPAVLQSDLY |
| | | TLSSSVTVPSSPRPSETVTCNVAHPASSTKVDKKIVPRDCGCKPCICTVPEVSSVFIFPPK |
| | | PKDVLTITLTPKVTCVVVDISKDDPEVQFSWFVDDVEVHTAQTQPREEQFNSTFRSVSELP |
| | | IMHQDWLNGKEFKCRVNSAAFPAPIEKTISKTKGRPKAPQVYTIPPPKEQMAKDKVSLTCM |
| | | ITDFFPEDITVEWQWNGQPAENYKNTQPIMNTNGSYFVYSKLNVQKSNWEAGNTFTCSVLH |
| | | EGLHNHHTEKSLSHSPGK |
| | | (SEQ ID NO:18) |

In some embodiments, an anti-hTREM2 antibody comprises a light chain having an amino acid sequence according to any one of SEQ ID NOS: 13-15, or any amino acid sequence that contains one or more, e.g., one, two, three, four or more amino acid substitutions (e.g., conservative amino acid substitutions), deletions or insertions of no more than five, four, three, two, or one amino acids to any one of SEQ ID NOS: 13-15. Such substitutions, deletions, and insertions would retain significant anti-hTREM2 binding activity. In these and other embodiments, an anti-hTREM2 antibody comprises a heavy chain having an amino acid sequence according to any one of SEQ ID NOS: 16-18, or any amino acid sequence that contains one or more, e.g., one, two, three, four or more amino acid substitutions (e.g., conservative amino acid substitutions), deletions or insertions of no more than five, four, three, two, or one amino acids to any one of SEQ ID NOS: 16-18. Such substitutions, deletions, and insertions would retain significant anti-hTREM2 binding activity.

In some embodiments, an anti-hTREM2 antibody comprises a light chain having an amino acid sequence according to SEQ ID NO: 13, and/or a heavy chain variable region having an amino acid sequence according to SEQ ID NO: 16. In other embodiments, an anti-hTREM2 antibody comprises a light chain having an amino acid sequence according to SEQ ID NO: 14, and/or a heavy chain variable region having an amino acid sequence according to SEQ ID NO: 17. In other embodiments, an anti-hTREM2 antibody comprises a light chain having an amino acid sequence according to SEQ ID NO: 15, and/or a heavy chain variable region having an amino acid sequence according to SEQ ID NO: 18.

In certain embodiments, the anti-TREM2 antibody is hT2AB, which is an hTREM2 agonist comprising a light chain having an amino acid sequence according to SEQ ID NO: 13, and a heavy chain variable region having an amino acid sequence according to SEQ ID NO: 16. In certain embodiments, the anti-TREM2 antibody is hT2AB having N-terminal leader sequences, comprising a light chain having an amino acid sequence according to SEQ ID NO: 14, and a heavy chain variable region having an amino acid sequence according to SEQ ID NO: 17. In certain embodiments, the anti-TREM2 antibody is mT2AB, which is a chimera of hT2AB variable regions with murine kappa and IgG1 constant regions, comprising a light chain having an amino acid sequence according to SEQ ID NO: 14 or an effectorless variant thereof, and a heavy chain having an amino acid sequence according to SEQ ID NO: 17 or an effectorless variant thereof.

### Polynucleotides

In another aspect, the present disclosure provides polynucleotides encoding the antibodies or antigen binding regions of the described herein. In particular, the polynucleotides are isolated polynucleotides. The polynucleotides may be operatively linked to one or more heterologous control sequences that control gene expression to create a recombinant polynucleotide capable of expressing the polypeptide of interest. Expression constructs containing a heterologous polynucleotide encoding the relevant polypeptide or protein can be introduced into appropriate host cells to express the corresponding polypeptide.

As will be appreciated by those in the art, due to the degeneracy of the genetic code, where the same amino acids are encoded by alternative or synonymous codons, an extremely large number of nucleic acids can be made, all of which encode the CDRs, variable regions, and heavy and light chains or other components of the antigen binding proteins described herein. Thus, having identified a particular amino acid sequence, those skilled in the art could make any number of different nucleic acids, by simply modifying the sequence of one or more codons in a way which does not change the amino acid sequence of the encoded protein. In this regard, the present disclosure includes each and every possible variation of polynucleotides that encode the polypeptides disclosed herein.

An “isolated nucleic acid,” which is used interchangeably herein with “isolated polynucleotide,” is a nucleic acid that has been separated from adjacent genetic sequences present in the genome of the organism from which the nucleic acid was isolated, in the case of nucleic acids isolated from naturally occurring sources. In the case of nucleic acids synthesized enzymatically from a template or chemically, such as PCR products, cDNA molecules, or oligonucleotides, for example, it is understood that the nucleic acids resulting from such processes are isolated nucleic acids. An isolated nucleic acid molecule refers to a nucleic acid molecule in the form of a separate fragment or as a component of a larger nucleic acid construct. In one preferred embodiment, the nucleic acids are substantially free from contaminating endogenous material.

In some embodiments, the polynucleotide encodes a CDRL1, CDRL2 and CDRL3 of a light chain variable region described herein. In some embodiments, the polynucleotide encodes a CDRH1, CDRH2 and CDRH3 of a heavy chain variable region described herein.

In some embodiments, the polynucleotide encodes a CDRL1, CDRL2 and CDRL3 of a light chain variable region and a CDRH1, CDRH2 and CDRH3 of a heavy chain variable region described herein.

In some embodiments, the polynucleotide encodes a light chain variable region VL having at least 80%, 85%, 90%, 91%, 92%, 93%, 94%, 95%, 96%, 97%, 98%, 99%, or greater sequence identity to the amino acid sequence of a variable light chain disclosed herein.

In some embodiments, the polynucleotide encodes a heavy chain variable region VH having at least 80%, 85%, 90%, 91%, 92%, 93%, 94%, 95%, 96%, 97%, 98%, 99%, or greater sequence identity to the amino acid sequence of a variable heavy chain disclosed herein.

In some embodiments, the polynucleotides herein may be manipulated in a variety of ways to provide for expression of the encoded polypeptide. In some embodiments, the polynucleotide is operably linked to control sequences, including among others, transcription promoters, leader sequences, transcription enhancers, ribosome binding or entry sites, termination sequences, and polyadenylation sequences, for expression of the polynucleotide and/or corresponding polypeptide. Manipulation of the isolated polynucleotide prior to its insertion into a vector may be desirable or necessary depending on the expression vector. The techniques for modifying polynucleotides and nucleic acid sequences utilizing recombinant DNA methods are well known in the art. Guidance is provided in Sambrook et al., Molecular Cloning: A Laboratory Manual, 3rd Ed., Cold Spring Harbor Laboratory Press (2001); and Current Protocols in Molecular Biology, Ausubel. F. ed., Greene Pub. Associates (1998), updates to 2013.

In some embodiments, variants of the antigen binding proteins, including the variants described herein, can be prepared by site-specific mutagenesis of nucleotides in the DNA encoding the polypeptide, using cassette or PCR mutagenesis or other techniques well known in the art, to produce DNA encoding the variant, and thereafter expressing the recombinant DNA in cell culture as outlined herein. However, antigen binding proteins comprising variant CDRs having up to about 100-150 residues may be prepared by in vitro synthesis using established techniques. The variants typically exhibit the same qualitative biological activity as the naturally occurring analogue, e.g., binding to antigen. Such variants include, for example, deletions and/or insertions and/or substitutions of residues within the amino acid sequences of the antigen binding proteins. Any combination of deletion, insertion, and substitution is made to arrive at the final construct, provided that the final construct possesses the desired characteristics. The amino acid changes also may alter post-translational processes of the antigen binding protein, such as changing the number or position of glycosylation sites. In some embodiments, antigen binding protein variants are prepared with the intent to modify those amino acid residues which are directly involved in epitope binding. In other embodiments, modification of residues which are not directly involved in epitope binding or residues not involved in epitope binding in any way, is desirable, for purposes discussed herein. Mutagenesis within any of the CDR regions, framework regions, and/or constant regions is contemplated. Covariance analysis techniques can be employed by the skilled artisan to design useful modifications in the amino acid sequence of the antigen binding protein. See, e.g., Choulier, et al., Proteins 41:475-484, 2000; Demarest et al., J. Mol. Biol., 2004, 335:41-48; Hugo et al., Protein Engineering, 2003, 16(5):381-86; Aurora et al., US Patent Publication No. 2008/0318207 A1; Glaser et al., US Patent Publication No. 2009/0048122 A1; Urech et al., WO 2008/110348 A1; Borras et al., WO 2009/000099 A2. Such modifications determined by covariance analysis can improve potency, pharmacokinetic, pharmacodynamic, and/or manufacturability characteristics of an antigen binding protein.

In another aspect, the present invention also provides vectors comprising one or more nucleic acids or polynucleotides encoding one or more components of the antigen binding proteins describe herein (e.g., variable regions, light chains, and heavy chains). As used herein, the term “vector” refers to any molecule or entity (e.g., nucleic acid, plasmid, bacteriophage, or virus) used to transfer protein coding information into a host cell. Examples of vectors include, but are not limited to, plasmids, viral vectors, non-episomal mammalian vectors, and expression vectors, for example, recombinant expression vectors. The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site, and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired.

The recombinant expression vector may be any vector (e.g., a plasmid or virus), which can be conveniently subjected to recombinant DNA procedures and can bring about the expression of the polynucleotide sequence. The choice of the vector will typically depend on the compatibility of the vector with the host cell into which the vector is to be introduced. The vectors may be linear or closed circular plasmids. Exemplary expression vectors include, among others, vectors based on T7 or T71ac promoters (pACY: Novagen; pET); vectors based on Baculovirus promoters (e.g., pBAC); vectors based on Ef1-α and HTLV promoters (e.g., pFUSE2; Invitrogen, CA, USA); vectors based on CMV enhancer and human ferritin light chain gene promoters (e.g., pFUSE: Invitrogen, CA, USA); vectors based on CMV promoters (e.g, pFLAG: Sigma, USA); and vectors based on dihydrofolate reductase promoters (e.g., pEASE: Amgen, USA). Various vectors can be used for transient or stable expression of the polypeptides of interest.

### Host Cells

In another aspect, the polynucleotide encoding the antigen binding proteins described herein (e.g., variable regions, light chains, and heavy chains) is operatively linked to one or more control sequences for expression of the polypeptide in the host cell. Accordingly, in a further aspect, the present disclosure provides a host cell comprising one or more expression vectors encoding the components of the TREM2 agonist antigen binding proteins described herein.

Exemplary host cells include prokaryote, yeast, or higher eukaryote cells. Prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces. Eukaryotic microbes such as filamentous fungi or yeast are suitable cloning or expression hosts for recombinant polypeptides. Saccharomyces cerevisiae, or common baker's yeast, is the most commonly used among lower eukaryotic host microorganisms. However, a number of other genera, species, and strains are commonly available and useful herein, such as Pichia, e.g., P. pastoris, Schizosaccharomyces pombe; Kluyveromyces, Yarrowia; Candida; Trichoderma reesia; Neurospora crassa; Schwanniomyces, such as Schwanniomyces occidentalis; and filamentous fungi, such as, e.g., Neurospora, Penicillium, Tolypocladium, and Aspergillus hosts such as A. nidulans and A. niger.

Host cells for the expression of glycosylated antigen binding proteins can be derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection of such cells are publicly available, e.g., the L-1 variant of Autographa californica NPV and the Bm-5 strain of Bombyx mori NPV.

Vertebrate host cells are also suitable hosts, and recombinant production of antigen binding proteins from such cells has become routine procedure. Mammalian cell lines available as hosts for expression are well known in the art and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA, 1980, 77: 4216); monkey kidney CV1 line transformed by SV40 (COS-7, ATCC CRL 1651); human embryonic kidney line (293 or 293 cells subcloned for growth in suspension culture) (Graham et al., J. Gen Virol. 36: 59, 1977); baby hamster kidney cells (BHK, ATCC CCL 10); mouse sertoli cells (TM4, Mather, Biol. Reprod., 1980, 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci., 1982, 383:44-68); MRC 5 cells or FS4 cells; mammalian myeloma cells, and a number of other cell lines. In certain embodiments, cell lines may be selected through determining which cell lines have high expression levels and constitutively produce antigen binding proteins with human TREM2 binding properties. In another embodiment, a cell line from the B cell lineage that does not make its own antibody but has a capacity to make and secrete a heterologous antibody can be selected. CHO cells are preferred host cells in some embodiments for expressing the TREM2 agonist antigen binding proteins of the invention.

In various embodiments, introduction and transformation of a host cell with a polynucleotide of the present disclosure, such as an expression vector for expressing an antigen binding protein, is accomplished by methods that including transfection, infection, calcium phosphate co-precipitation, electroporation, microinjection, lipofection, DEAE-dextran mediated transfection, or other known techniques. In some embodiments, the method selected can be guided by the type of host cell used. Suitable methods are described in, for example, Sambrook et al., 2001.

### Expression and Isolation

In some embodiments, the host cell comprising a polynucleotide encoding one or more components of the antigen binding proteins described herein (e.g., variable regions, light chains, and heavy chains) is used to express the antigen binding protein of interest. In some embodiments, a method for expressing the antigen binding protein comprises culturing the host cell in suitable media and conditions appropriate for expression of the protein of interest.

The type of media and culture conditions selected is based on the type of host cell. In some embodiments, exemplary media for mammalian host cells include, by way of example and not limitation, Ham's F10 (Sigma), Minimal Essential Medium (MEM, Sigma), RPMI-1640 (Sigma), and Dulbecco's Modified Eagle's Medium (DMEM, Sigma). In some embodiments, the media can be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. In some embodiments, culture conditions, such as temperature, pH, % CO₂, and the like, can use conditions available and known to the skilled artisan.

In some embodiments, the expressed antigen binding protein is isolate and/or purified from the host cell. In some embodiments in which the expressed protein in present in the media, the media containing the expressed protein is subject to isolation procedures. In some embodiments in which the antigen binding protein is produced intracellularly, the cells are subject to disruption, and as a first step, the particulate debris, either host cells or lysed fragments, is removed, for example, by centrifugation or ultrafiltration. Subsequently, the antigen binding protein can be isolated and further purified by various known techniques. Such isolation techniques include affinity chromatography with Protein-A Sepharose, size-exclusion chromatography, ion-exchange chromatography, high performance liquid chromatography, differential solubility, and the like (see, e.g., Fisher, Laboratory Techniques, In Biochemistry And Molecular Biology, Work and Burdon, eds., Elsevier (1980); Antibodies: A Laboratory Manual, Greenfield, E. A., ed., Cold Spring Harbor Laboratory Press, New York (2012); Coligan, et al., supra, sections 2.7.1-2.7.12 and sections 2.9.1-2.9.3; Barnes, et al., Purification of Immunoglobulin G (IgG), in Methods Mol. Biol., Vol. 10, pages 79-104, Humana Press (1992)).

In some embodiments, the isolated antibody can be further purified as measurable by: (1) weight of protein as determined using the Lowry method; (2) to a degree sufficient to obtain at least 15 residues of N-terminal or internal amino acid sequence by use of a spinning-cup sequencer; or (3) to homogeneity by SDS-PAGE under reducing or non-reducing conditions using Coomassie blue or, preferably, silver stain. The purified antibody can be 85% or greater, 90% or greater, 95% or greater, or at least 99% by weight as determined by the foregoing methods.

### Antibody Formulations

In certain embodiments, the invention provides a composition (e.g., a pharmaceutical composition) comprising one or a plurality of the TREM2 activating antibodies and TREM2 agonist antibodies and antigen binding proteins disclosed herein together with pharmaceutically acceptable diluents, carriers, excipients, solubilizers, emulsifiers, preservatives, and/or adjuvants. Pharmaceutical compositions of the invention include, but are not limited to, liquid, frozen, and lyophilized compositions. “Pharmaceutically-acceptable” refers to molecules, compounds, and compositions that are non-toxic to human recipients at the dosages and concentrations employed and/or do not produce allergic or adverse reactions when administered to humans. In some embodiments, the pharmaceutical composition may contain formulation materials for modifying, maintaining, or preserving, for example, the pH, osmolarity, viscosity, clarity, color, isotonicity, odor, sterility, stability, rate of dissolution or release, adsorption, or penetration of the composition. In such embodiments, suitable formulation materials include, but are not limited to, amino acids (such as glycine, glutamine, asparagine, arginine, or lysine); antimicrobials; antioxidants (such as ascorbic acid, sodium sulfite, or sodium hydrogen-sulfite); buffers (such as borate, bicarbonate, Tris-HCl, citrates, phosphates, or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin, or hydroxypropyl-beta-cyclodextrin); fillers; monosaccharides; disaccharides; and other carbohydrates (such as glucose, mannose, or dextrins); proteins (such as serum albumin, gelatin, or immunoglobulins); coloring, flavoring, and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid, or hydrogen peroxide); solvents (such as glycerin, propylene glycol, or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients, and/or pharmaceutical adjuvants. Methods and suitable materials for formulating molecules for therapeutic use are known in the pharmaceutical arts, and are described, for example, in Remington's Pharmaceutical Sciences, 18th Ed., (A. R. Genrmo, ed.), 1990, Mack Publishing Company.

In some embodiments, the pharmaceutical composition of the invention comprises a standard pharmaceutical carrier, such as a sterile phosphate buffered saline solution, bacteriostatic water, and the like. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like.

Exemplary concentrations of the antigen binding proteins in the formulation may range from about 0.1 mg/ml to about 200 mg/ml or from about 0.1 mg/mL to about 50 mg/mL, or from about 0.5 mg/mL to about 25 mg/mL, or alternatively from about 2 mg/mL to about 10 mg/mL. An aqueous formulation of the antigen binding protein may be prepared in a pH-buffered solution, for example, at pH ranging from about 4.5 to about 6.5, or from about 4.8 to about 5.5, or alternatively about 5.0. Examples of buffers that are suitable for a pH within this range include acetate (e.g., sodium acetate), succinate (such as sodium succinate), gluconate, histidine, citrate, and other organic acid buffers. The buffer concentration can be from about 1 mM to about 200 mM, or from about 10 mM to about 60 mM, depending, for example, on the buffer and the desired isotonicity of the formulation.

A tonicity agent, which may also stabilize the antigen binding protein, may be included in the formulation. Exemplary tonicity agents include polyols, such as mannitol, sucrose, or trehalose. Preferably the aqueous formulation is isotonic, although hypertonic or hypotonic solutions may be suitable. Exemplary concentrations of the polyol in the formulation may range from about 1% to about 15% w/v.

A surfactant may also be added to the antigen binding protein formulation to reduce aggregation of the formulated antigen binding protein and/or minimize the formation of particulates in the formulation and/or reduce adsorption. Exemplary surfactants include nonionic surfactants such as polysorbates (e.g., polysorbate 20 or polysorbate 80) or poloxamers (e.g., poloxamer 188). Exemplary concentrations of surfactant may range from about 0.001% to about 0.5%, or from about 0.005% to about 0.2%, or alternatively from about 0.004% to about 0.010% w/v.

In one embodiment, the formulation contains the above-identified agents (i.e., antigen binding protein, buffer, polyol, and surfactant) and is essentially free of one or more preservatives, such as benzyl alcohol, phenol, m-cresol, chlorobutanol, and benzethonium chloride. In another embodiment, a preservative may be included in the formulation, e.g., at concentrations ranging from about 0.1% to about 2%, or alternatively from about 0.5% to about 1%. One or more other pharmaceutically acceptable carriers, excipients, or stabilizers such as those described in REMINGTON'S PHARMACEUTICAL SCIENCES, 18th Edition, (A. R. Genrmo, ed.), 1990, Mack Publishing Company, may be included in the formulation provided that they do not adversely affect the desired characteristics of the formulation.

Therapeutic formulations of the antigen binding protein are prepared for storage by mixing the antigen binding protein having the desired degree of purity with optional physiologically acceptable carriers, excipients, or stabilizers (Remington's Pharmaceutical Sciences, 18th Ed., (A. R. Genrmo, ed.), 1990, Mack Publishing Company), in the form of lyophilized formulations or aqueous solutions. Acceptable carriers, excipients, or stabilizers are nontoxic to recipients at the dosages and concentrations employed, and include buffers (e.g., phosphate, citrate, and other organic acids); antioxidants (e.g., ascorbic acid and methionine); preservatives (such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol; resorcinol, cyclohexanol, 3-pentanol, and m-cresol); low molecular weight (e.g., less than about 10 residues) polypeptides; proteins (such as serum albumin, gelatin, or immunoglobulins); hydrophilic polymers (e.g., polyvinylpyrrolidone); amino acids (e.g., glycine, glutamine, asparagine, histidine, arginine, or lysine); monosaccharides, disaccharides, and other carbohydrates including glucose, mannose, maltose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants, such as polysorbates (e.g., polysorbate 20 or polysorbate 80) or poloxamers (e.g., poloxamer 188); or polyethylene glycol (PEG).

In one embodiment, a suitable formulation of the claimed invention contains an isotonic buffer such as a phosphate, acetate, or TRIS buffer in combination with a tonicity agent, such as a polyol, sorbitol, sucrose, or sodium chloride, which tonicifies and stabilizes. One example of such a tonicity agent is 5% sorbitol or sucrose. In addition, the formulation could optionally include a surfactant at 0.01% to 0.02% wt/vol, for example, to prevent aggregation or improve stability. The pH of the formulation may range from 4.5 to 6.5 or 4.5 to 5.5. Other exemplary descriptions of pharmaceutical formulations for antigen binding proteins may be found in US Patent Publication No. 2003/0113316 and U.S. Pat. No. 6,171,586, each of which is hereby incorporated by reference in its entirety.

Suspensions and crystal forms of antigen binding proteins are also contemplated. Methods to make suspensions and crystal forms are known to one of skill in the art.

The formulations to be used for in vivo administration must be sterile. The compositions of the invention may be sterilized by conventional, well-known sterilization techniques. For example, sterilization is readily accomplished by filtration through sterile filtration membranes. The resulting solutions may be packaged for use or filtered under aseptic conditions and lyophilized, the lyophilized preparation being combined with a sterile solution prior to administration.

The process of freeze-drying is often employed to stabilize polypeptides for long-term storage, particularly when the polypeptide is relatively unstable in liquid compositions. A lyophilization cycle is usually composed of three steps: freezing, primary drying, and secondary drying (see Williams and Polli, Journal of Parenteral Science and Technology, 1984, 38(2):48-59). In the freezing step, the solution is cooled until it is adequately frozen. Bulk water in the solution forms ice at this stage. The ice sublimes in the primary drying stage, which is conducted by reducing chamber pressure below the vapor pressure of the ice, using a vacuum. Finally, sorbed or bound water is removed at the secondary drying stage under reduced chamber pressure and an elevated shelf temperature. The process produces a material known as a lyophilized cake. Thereafter the cake can be reconstituted prior to use.

The standard reconstitution practice for lyophilized material is to add back a volume of pure water (typically equivalent to the volume removed during lyophilization), although dilute solutions of antibacterial agents are sometimes used in the production of pharmaceuticals for parenteral administration (see Chen, Drug Development and Industrial Pharmacy, Volume 18: 1311-1354, 1992).

Excipients have been noted in some cases to act as stabilizers for freeze-dried products (see Carpenter et al., Volume 74: 225-239, 1991). For example, known excipients include polyols (including mannitol, sorbitol, and glycerol); sugars (including glucose and sucrose); and amino acids (including alanine, glycine, and glutamic acid).

In addition, polyols and sugars are also often used to protect polypeptides from freezing and drying-induced damage and to enhance the stability during storage in the dried state. In general, sugars, in particular disaccharides, are effective in both the freeze-drying process and during storage. Other classes of molecules, including mono- and di-saccharides, and polymers such as PVP, have also been reported as stabilizers of lyophilized products.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Sustained-release preparations may be prepared. Suitable examples of sustained-release preparations include semipermeable matrices of solid hydrophobic polymers containing the antigen binding protein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The formulations of the invention may be designed to be short-acting, fast-releasing, long-acting, or sustained-releasing. Thus, the pharmaceutical formulations may also be formulated for controlled release or for slow release.

Specific dosages may be adjusted depending on the disease, disorder, or condition to be treated, the age, body weight, general health conditions, sex, and diet of the subject, dose intervals, administration routes, excretion rate, and combinations of drugs.

The TREM2 agonist antigen binding proteins of the invention can be administered by any suitable means, including parenteral, subcutaneous, intraperitoneal, intrapulmonary, intrathecal, intracerebral, intracerebroventricular, and intranasal, and, if desired for local treatment, intralesional administration. Parenteral administration includes intravenous, intraarterial, intraperitoneal, intramuscular, intradermal, or subcutaneous administration. In addition, the antigen binding protein is suitably administered by pulse infusion, particularly with declining doses of the antigen binding protein. Preferably, the dosing is given by injections, most preferably intravenous or subcutaneous injections, depending in part on whether the administration is brief or chronic. Other administration methods are contemplated, including topical, particularly transdermal, transmucosal, rectal, oral, or local administration, e.g., through a catheter placed close to the desired site. In certain embodiments, the TREM2 agonist antigen binding protein of the invention is administered intravenously or subcutaneously in a physiological solution at a dose ranging between 0.01 mg/kg to 100 mg/kg at a frequency ranging from daily to weekly to monthly (e.g., every day, every other day, every third day, or 2, 3, 4, 5, or 6 times per week), preferably a dose ranging from 0.1 to 45 mg/kg, 0.1 to 15 mg/kg or 0.1 to 10 mg/kg at a frequency of once per week, once every two weeks, or once a month.

The TREM2 agonist antigen binding proteins described herein (e.g., anti-TREM2 agonist monoclonal antibodies and binding fragments thereof) are useful for preventing, treating, or ameliorating a condition associated with TREM2 deficiency or loss of biological function of TREM2 in a patient in need thereof. As used herein, the term “treating” or “treatment” is an intervention performed with the intention of preventing the development or altering the pathology of a disorder. Accordingly, “treatment” refers to both therapeutic treatment and prophylactic or preventative measures. Patients in need of treatment include those already diagnosed with or suffering from the disorder or condition as well as those in which the disorder or condition is to be prevented, such as patients who are at risk of developing the disorder or condition based on, for example, genetic markers. “Treatment” includes any indicia of success in the amelioration of an injury, pathology, or condition, including any objective or subjective parameter such as abatement, remission, diminishing of symptoms, or making the injury, pathology, or condition, more tolerable to the patient, slowing in the rate of degeneration or decline, making the final point of degeneration less debilitating, or improving a patient's physical or mental well-being. The treatment or amelioration of symptoms can be based on objective or subjective parameters, including the results of a physical examination, self-reporting by a patient, cognitive tests, motor function tests, neuropsychiatric exams, and/or a psychiatric evaluation.

### 7.8.2. Small Molecule TREM2 Agonists

### Small Molecule Agonists

In any of the aforementioned embodiments, the TREM2 agonist used in the various methods and assays of the invention can be a small molecule TREM2 agonist, or a salt thereof.

In some embodiments, the agonist of TREM2 is a lipid ligand of TREM2. In some embodiments, the lipid ligand of TREM2 is selected from 1-palmitoyl-2-(5′-oxo-valeroyl)-sn-glycero-3-phosphocholine (POVPC), 2-Arachidonoylglycerol (2-AG), 7-ketocholesterol (7-KC), 24(S)hydroxycholesterol (240HC), 25(S)hydroxycholesterol (250HC), 27-hydroxycholesterol (270HC), Acyl Carnitine (AC), alkylacylglycerophosphocholine (PAF), a-galactosylceramide (KRN7000), Bis(monoacylglycero)phosphate (BMP), Cardiolipin (CL), Ceramide, Ceramide-1-phosphate (CIP), Cholesteryl ester (CE), Cholesterol phosphate (CP), Diacylglycerol 34: 1 (DG 34: 1), Diacylglycerol 38:4 (DG 38:4), Diacylglycerol pyrophosphate (DGPP), Dihyrdoceramide (DhCer), Dihydrosphingomyelin (DhSM), Ether phosphatidylcholine (PCe), Free cholesterol (FC), Galactosylceramide (GalCer), Galactosylsphingosine (GalSo), Ganglioside GM1, Ganglioside GM3, Glucosylsphingosine (GlcSo), Hank's Balanced Salt Solution (HBSS), Kdo2-Lipid A (KLA), Lactosylceramide (LacCer), lysoalkylacylglycerophosphocholine (LPAF), Lysophosphatidic acid (LPA), Lysophosphatidylcholine (LPC), Lysophosphatidylethanolamine (LPE), Lysophosphatidylglycerol (LPG), Lysophosphatidylinositol (LPI), Lysosphingomyelin (LSM), Lysophosphatidylserine (LPS), N-Acyl-phosphatidylethanolamine (NAPE), N-Acyl- Serine (NSer), Oxidized phosphatidylcholine (oxPC), Palmitic-acid-9-hydroxy-stearic-acid (PAHSA), Phosphatidylethanolamine (PE), Phosphatidylethanol (PEtOH), Phosphatidic acid (PA), Phosphatidylcholine (PC), Phosphatidylglycerol (PG), Phosphatidylinositol (PI), Phosphatidylserine (PS), Sphinganine, Sphinganine-1-phosphate (SalP), Sphingomyelin (SM), Sphingosine, Sphingosine-1-phosphate (SolP), or Sulfatide, or a salt thereof.

In some embodiments, the agonist of TREM2 is a lipopolysaccharide.

In some embodiments, the agonist of TREM2 is a small molecule disclosed in PCT Application Publication WO2019/079529, which is incorporated by reference herein in its entirety. In some embodiments, the agonist of TREM2 is Tyrphostin AG 538, AC1NS458, IN1040, Butein, Okanin, AGL 2263, GB19, GB16, GB20, GB17, GB18, GB21, GB22, GB27, GB44, GB42, GB2, 4,4′-Dihydroxychalcone, or 3,4-Dihydroxybenzophenone, or a derivative or salt of any of the aforementioned.

In some embodiments, the agonist of TREM2 is a small molecule identified by a method disclosed in PCT Application Publication WO2019/079529. In some embodiments, the small molecule agonist of TREM2 is identified by applying the small molecule compound to a host cell expressing TREM2 and tyrosine kinase binding protein (TYROBP), wherein the host cell has a synthetic sequence comprising an NFAT-response element and a nucleotide sequence encoding a reporter, and measuring a signal emitted by the reporter.

In some embodiments, the agonist of TREM2 is a TREM2 agonist compound comprising a bicyclic core. In some embodiments, the bicyclic core is a 10-membered heteroaryl core. In some embodiments, the TREM2 agonist compound comprises a 10-membered heteroaryl core, comprising 1-4 nitrogen atoms as part of the core ring structure. In some embodiments, the TREM2 agonist compound comprises a 10-membered heteroaryl core, comprising 3 or 4 substituent groups.

### 8. EXAMPLES

The following Examples, which highlight certain features and properties of the exemplary embodiments of the antibodies and binding fragments described herein are provided for purposes of illustration, and not limitation.

### Example 1. Nanostring® Analysis of Gene Expression Profiles in a Mouse Model

Twenty-five adult male mice expressing human TREM2 (common variant of TREM2, hTREM2-CV), with homozygous murine TREM2 knockout were used as a model of human TREM2 deficiency. The hTREM2-CV mice were dosed with either a murinized anti-hTREM2 antibody (“Ab-3”) with a single dose, or a small molecule agonist of TREM2 (“Compound X”), in two doses (BID:0h and 10h) and then euthanized for tissue collection at 24h, to measure altered gene expression by Nanostring® Analysis.

### Antibody Ab-3

Antibody Ab-3 is a murinized version of a human TREM2 agonist antibody, first described as an engineered variant of antibody 13E7 in PCT Application Publication WO2018/195506A1. Ab-3 has an HC according to SEQ ID NO: 19, an LC according to SEQ ID NO:20 (as shown in Table 3), and exemplifies an anti-TREM2 antibody having the CDRs according to SEQ ID NOS:5-8 and 9-10.

TABLE 3
Murinized Anti-TREM2 Antibody Ab-3 Sequences

| Sequence | |
|---|---|
| Description | Amino Acid Sequence |
| Ab-3 HC | EVQLVQSGAEVKKPGESLKISCKGSGYSFTSYWIGWV |
| SEQ ID | RQMPGKGLEWMGIIYPGDADARYSPSFQGQVTISADK |
| NO: 19 | SISTAYLQWSSLKASDTAMYFCARRRQGIFGDALDFW |
| | GQGTLVTVSSAKTTPPSVYPLAPGSAAQTNSMVTLGC |
| | LVKGYFPEPVTVTWNSGSLSSGVHTFPAVLQSDLYTL |
| | SSSVTVPSSTWPSETVTCNVAHPASSTKVDKKIVPRD |
| | CGCKPCICTVPEVSSVFIFPPKPKDVLTITLTPKVTC |
| | VVVDISKDDPEVQFSWFVDDVEVHTAQTQPREEQFGS |
| | TFRSVSELPIMHQDWLNGKEFKCRVNSAAFPAPIEKT |
| | ISKTKGRPKAPQVYTIPPPKEQMAKDKVSLTCMITDF |
| | FPEDITVEWQWNGQPAENYKNTQPIMDTDGSYFVYSK |
| | LNVQKSNWEAGNTFTCSVLHEGLHNHHTEKSLSHSPG |
| | K |
| Ab-3 LC | EIVMTQSPATLSVSPGERATLSCRASQSVSSNLAWFQ |
| SEQ ID | QKPGQAPRLLIYGASTRATGIPARFSGSGSGTEFTLT |
| NO: 20 | ISSLQPEDFAVYYCLQDNNFPPTFGQGTKVDIKRADA |
| | APTVSIFPPSSEQLTSGGASVVCFLNNFYPKDINVKW |
| | KIDGSERQNGVLNSWTDQDSKDSTYSMSSTLTLTKDE |
| | YERHNSYTCEATHKTSTSPIVKSFNRNEC |

### Compound X

Compound X is a small molecule agonist of TREM2. Compound X has a 10-membered heteroaryl core, containing 3 nitrogen atoms as part of the core ring structure, and has four substituents off of the central core structure.

### Animal Care and Dosing Procedures

Animals were group-housed with access to food and water ad libitum. Animals were maintained on a 12:12 hour reverse light cycle with constant room temperature- (22±2° C.) and humidity (approx. 50%). Experiments were conducted in accordance with protocols approved by the IACUC of Charles River Laboratories, SSF. 6 animals received Ab-3 administered IP at 100 mpk, while an additional 6 animals received Compound X administered by oral gavage at 50 mpk BID, using the timing described above. An additional 12 animals received controls; 6 received a solution 2% Hydroxypropyl Methylcellulose, 1% Tween-80 in PBS by oral gavage and 6 received 100 mpk of a non-binding matched IgG isotype control by IP. Animals were dosed twice with their respective test compounds at time points 0 h and 10 h. Twenty-four hours after dosing, animals were euthanized for tissue collection by isoflurane overdose. Terminal blood samples were collected via cardiac puncture into K3 EDTA anticoagulant vials and processed for plasma. After centrifugation (2500 g for 10 min at +4° C.), plasma was aliquoted and stored at −80° C. Animals were then perfused with PBS and brain regions were dissected out, snap frozen, and stored at −80° C.

### Sample Collection and Analysis

RNA was extracted from flash frozen right hippocampal tissue samples using Qiagen reagents, and concentration was determined using Qubit. RNA quality was determined using Agilent Bioanalyzer, with samples having a RIN of >7 proceeding to Nanostring® Analysis. 100 ng of each RNA sample was hybridized to Nanostring® Mouse Neuroinflammation probe sets according to the manufacturer's protocol. After overnight hybridization, samples were loaded on Sprint cartridges, and scanned using the Nanostring® Sprint system. Key gene expression profiles were analyzed using the nCounter® Murine Neuroinflammation panel of 770 genes related to inflammation in the CNS. Results of individual gene expression changes relative to the mean of several housekeeper genes were grouped in pathways of interest and assigned a relative score using the nSolver analysis software.

Nanostring® files were analyzed according to manufacturer's recommended methods. Genes were normalized against the geometric mean of 12 housekeeping genes, and genes determined to be within the noise threshold were assigned a count value equal to the negative control. Noise cutoff was calculated by determining the maximum value of the (average+(2× standard deviation)) of the negative controls for each sample. Pathway analysis, cell type population analysis, and differential gene expression were all generated using the Nanostring® Advanced Analysis software, according to manufacturer's recommended protocols.

Nanostring® nSolver™ software includes a module for Cell Type Profiling that identifies genes linked to cell type in an experiment. Analysis using this module revealed an increase in microglia score (microglial-associated genes) with treatment with Compound X and Ab-3, but no change in neuron or astrocyte scores, indicating microglial-specific effects of TREM2 agonism by both treatments, as expected with TREM2 activation. Table 4 reports the Cell Type Profiling scores for microglia, neuron, and astrocyte genes, showing that microglia genes were upregulated by treatment with Compound X and Ab-3. Pathway analysis was also performed. Genes associated with the adaptive immune response, innate immune response, microglia function, cytokine signaling, and cell cycle were all increased in hippocampi from animals treated with Compound X and Ab-3. Table 5 reports the effects of Compound X and Ab-3 on these genes, where the values reflect PC1 scores from principal component analysis of the gene set. These results support the finding of TREM2 target engagement on microglia using Compound X and Ab-3.

TABLE 4
Cell Type Profiling Scores after treatment

| | Treatment Group | | |
|---|---|---|---|
| Gene Type | Vehicle | Cmpd X | Ab-3 |
| Microglia Score | 6.59 (0.108) | 6.82 (0.090) * | 7.01 (0.097) * |
| Neuron Score | 8.82 (0.108) | 8.81 (0.185) | 8.76 (0.063) |
| Astrocyte Score | 8.82 (0.044) | 8.86 (0.077) | 8.78 (0.044) |

Data shown is mean score (standard deviation).
* = p < 0.005 by Student's T-test, two-tailed.

TABLE 5
Cell Type Profiling Scores after treatment

| | Treatment Group | | |
|---|---|---|---|
| Pathway Score | Vehicle | Cmpd X | Ab-3 |
| Adaptive | −1.01 (0.594) | 0.639 (0.929) * | 1.66 (0.400) * |
| Immune | | | |
| Response | | | |
| Innate Immune | −0.984 (0.585) | 0.490 (0.674) * | 1.77 (0.418) * |
| Response | | | |
| Microglia | −1.04 (0.827) | 0.345 (0.908) * | 2.07 (0.424) * |
| Function | | | |
| Cytokine | −0.821 (0.564) | 0.180 (0.514) * | 1.75 (0.320) * |
| Signaling | | | |
| Cell Cycle | −0.789 (0.351) | 0.398 (0.414) * | 1.41 (0.462) * |

Data shown is mean score (standard deviation).
* = p < 0.005 by Student's T-test, two-tailed.

As shown in Table 4, effects of both treatments on gene expression were specific to microglia. Only the microglial score increased with treatment, while no effect was observed on neurons or astrocytes. Because TREM2 is exclusively expressed on myeloid cells, this confirms that the effects of both Ab-3 and Compound X are specific to microglia, and that there is no off-target effect on these other types of neurological cells.

As shown in Table 5, both treatments showed an increase in pathways tied to activation of TREM2 signaling, including microglial function, innate immunity, growth factor signaling and cell cycle. The majority of available pathways on the mouse neuroinflammation probe set were affected by both treatments in a similar manner.

The effects on individual homeostatic genes from the larger probe set were also measured, both for the mice dosed with Ab-3 and those dosed with Compound X, using the Nanostring® Advanced Analysis Differential Expression module. Normalized counts from samples taken from the Ab-3, Compound X, and vehicle control RNA samples were generated and counts were normalized against the geometric mean of 12 housekeeping genes, as well as against positive controls for each sample. Genes determined to be within the noise threshold were assigned a count value equal to the negative control. Noise cutoff was calculated by determining the maximum value of the (average+(2× standard deviation)) of the negative controls for each sample. LOG 2-fold change was determined by the converting the ratio of treatment counts to control counts to log 2.

Table 6 reports a list of homeostatic genes, along with the log 2-fold change in gene expression relative to vehicle control after treatment with Ab-3 or Compound X. Genes that were not differentially expressed (Benjamini-Hochberg corrected p<0.10), or detected, are not shown in Table 6. Blank cells indicate that significant differential expression was not observed in that sample set for that gene.

TABLE 6
Genes Differentially Expressed After Treatment
with Ab-3 or Compound X.
Values are shown as the log2-fold change in
expression relative to the vehicle control.

| Gene | Ab-3 | Cmpd X |
|---|---|---|
| Adamts16 | −0.797 | −0.741 |
| AI464131 | −0.125 | |
| Apex1 | 0.136 | 0.104 |
| Apoe | | 0.123 |
| Arc | −0.478 | |
| Axl | 0.126 | |
| Atg14 | | 0.0982 |
| Bad | | 0.26 |
| Bard1 | 0.688 | 0.517 |
| Bcl2a1a | 1.04 | |
| Bcl2 | −0.413 | |
| Birc5 | 0.843 | |
| Blnk | 0.333 | |
| Bnip3l | | 0.0454 |
| Bnip3 | | 0.0895 |
| Btk | 0.624 | |
| C1qa | 0.658 | 0.315 |
| C1qb | 0.663 | 0.325 |
| C1qc | 0.624 | 0.346 |
| C3 | | 1.14 |
| C3ar1 | 0.437 | |
| C4a | 0.71 | |
| C5ar1 | 0.733 | |
| Cables1 | | −0.167 |
| Camk4 | −0.161 | −0.192 |
| Casp4 | 0.875 | |
| Casp6 | | 0.235 |
| Casp7 | 0.392 | |
| Casp8 | 0.4 | 0.487 |
| Ccl3 | 1.64 | |
| Ccr5 | 0.325 | |
| Cd14 | 1.02 | |
| Cd40 | | -0.493 |
| Cd68 | 0.632 | 0.309 |
| Cd72 | 1.63 | |
| Cd74 | | 1.26 |
| Cd83 | 0.218 | |
| Cd84 | 0.551 | |
| Cd86 | 0.452 | |
| Cdk20 | | 0.168 |
| Cdkn1c | | 1.37 |
| Cflar | | 0.333 |
| Cks1b | | 0.229 |
| Clic4 | | 0.233 |
| Clstn1 | −0.181 | −0.17 |
| Cnn2 | | 0.366 |
| Csf1 | 0.184 | 0.155 |
| Csf1r | 0.284 | 0.179 |
| Csf2rb | 0.478 | |
| Ctss | 0.259 | |
| Cx3cr1 | 0.198 | |
| Cxcl10 | 2.17 | |
| Dab2 | | 0.812 |
| Ddx58 | 0.513 | 0.475 |
| Dna2 | 0.33 | |
| Dock1 | | 0.163 |
| Dock2 | 0.702 | 0.339 |
| Dusp7 | | −0.0979 |
| E2f1 | 0.384 | |
| Eed | 0.0933 | 0.111 |
| Emcn | | 0.531 |
| Ercc2 | | 0.116 |
| Ezh2 | 0.225 | |
| Fcer1g | 0.618 | 0.305 |
| Fcgr1 | 0.604 | 0.328 |
| Fcrls | 0.493 | 0.342 |
| Fen1 | 0.27 | 0.17 |
| Fscn1 | -0.1 | −0.109 |
| Gal3st1 | -0.354 | |
| Gbp2 | 0.745 | 0.413 |
| Gna15 | 0.281 | |
| Gpr183 | 0.617 | |
| Gpr34 | 0.216 | 0.218 |
| Grm3 | | −0.0958 |
| Grn | 0.176 | 0.159 |
| Gsn | | 0.279 |
| Gstm1 | 0.146 | |
| Hdac1 | | 0.122 |
| Hdac4 | −0.157 | |
| Hells | 0.338 | |
| Homer1 | −0.177 | -0.203 |
| Hpgds | | 0.186 |
| Ifi30 | 0.934 | 0.555 |
| Ifih1 | 0.306 | |
| Ifitm2 | | 0.363 |
| Ifitm3 | 0.687 | 0.506 |
| Ifnar2 | 0.205 | 0.218 |
| Il10rb | | 0.339 |
| Il1a | 1.18 | |
| Il1rl2 | 0.701 | |
| Il2rg | 0.878 | |
| Inpp5d | 0.421 | |
| Iqsec1 | −0.0891 | |
| Irak1 | | 0.18 |
| Irf1 | 0.21 | 0.279 |
| Irf3 | | 0.148 |
| Irf7 | 0.907 | 0.41 |
| Irf8 | 0.419 | |
| Itgam | 0.36 | 0.271 |
| Itgav | | 0.152 |
| Itgb5 | 0.234 | 0.338 |
| Jun | 0.144 | |
| Kat2b | | 0.276 |
| Kcnj10 | | −0.139 |
| Kdm4d | | −0.527 |
| Kdm5c | | 0.103 |
| Lacc1 | 0.355 | |
| Lair1 | 0.436 | |
| Lamp1 | | 0.0973 |
| Lamp2 | | 0.34 |
| Lgmn | 0.305 | 0.292 |
| Lig1 | 0.397 | |
| Lilrb4a | 1.02 | |
| Lingo1 | | −0.189 |
| Lmnb1 | 0.246 | |
| Ltbr | | 0.375 |
| Ly6a | | −0.157 |
| Lyn | 0.22 | |
| Man2b1 | 0.157 | 0.197 |
| Map3k1 | | 0.185 |
| Map1lc3a | −0.167 | |
| Mapt | −0.0978 | −0.0718 |
| Mavs | | 0.219 |
| Mb21d1 | | 0.308 |
| Mbd2 | | 0.0943 |
| Mcm2 | 0.387 | |
| Mcm5 | 0.675 | |
| Mcm6 | 0.428 | |
| Mdm2 | | 0.122 |
| Mertk | | −0.151 |
| Mmp14 | | 0.266 |
| Mpeg1 | | 0.23 |
| Mr1 | | 0.593 |
| Msh2 | | 0.0819 |
| Msn | 0.332 | 0.324 |
| Mvp | | 0.37 |
| Ncaph | 0.617 | |
| Ncf1 | 0.551 | 0.277 |
| Nfkb1 | | 0.107 |
| Nfkb2 | 0.446 | |
| Ngf | −0.158 | |
| Nlgn2 | −0.106 | |
| Nostrin | | -0.251 |
| Nqo1 | | 0.879 |
| Olfml3 | 0.789 | 0.471 |
| Osmr | 0.326 | |
| P2rx7 | | 0.164 |
| P2ry12 | | 0.162 |
| Pacsin1 | −0.0908 | |
| Pak1 | −0.123 | −0.108 |
| Pcna | 0.242 | 0.152 |
| Pdpn | | 0.456 |
| Pik3cb | 0.126 | 0.09 |
| Pik3r2 | | −0.0808 |
| Pla2g4a | 0.457 | 0.492 |
| Pla2g5 | | 2 |
| Plcg2 | 0.366 | |
| Plxdc2 | 0.157 | 0.137 |
| Pole | 0.851 | |
| Prdx1 | 0.148 | |
| Prkcq | | 0.631 |
| Pros1 | 0.419 | 0.27 |
| Psmb8 | 0.82 | 0.538 |
| Ptpn6 | 0.947 | 0.471 |
| Ptprc | 0.481 | 0.351 |
| Rab6b | −0.0989 | −0.122 |
| Rac2 | 0.513 | |
| Rad1 | | 0.11 |
| Rad51 | 0.92 | 0.449 |
| Rapgef3 | | −0.128 |
| Rblcc1 | 0.196 | |
| Rela | | 0.15 |
| Rnf8 | | 0.0752 |
| Rp128 | | 0.0631 |
| Rpl36al | 0.0822 | |
| Rps2 | | 0.0698 |
| Rps9 | | 0.0992 |
| Rrm2 | 0.981 | |
| Rsad2 | 0.465 | |
| S1pr3 | | 0.276 |
| Sall1 | | 0.148 |
| Serpina3n | 0.253 | |
| Siglecf | 0.832 | |
| Sirt1 | | 0.121 |
| Slamf9 | 1.71 | |
| Slc6a1 | −0.158 | |
| Slco2b1 | 0.287 | |
| Slfn8 | 0.598 | 0.475 |
| Smc1a | 0.06 | |
| Sox9 | | 0.23 |
| Srgn | 0.339 | |
| Stat1 | 0.299 | 0.2 |
| Stx18 | | 0.188 |
| Suz12 | | 0.106 |
| Tcirg1 | | 0.184 |
| Tgfa | | 0.18 |
| Tgfb1 | 0.518 | 0.286 |
| Tgfbr1 | 0.362 | 0.16 |
| Tgm2 | | −0.285 |
| Tie1 | | −0.213 |
| Tlr4 | 0.419 | |
| Tm4sf1 | | 0.35 |
| Tmem119 | 0.461 | 0.242 |
| Tmem173 | 0.495 | |
| Tmem206 | | 0.125 |
| Tmem37 | | 0.76 |
| Tmem64 | | 0.125 |
| Tnfrsf12a | | 0.297 |
| Tnfrsf1a | 0.269 | 0.286 |
| Top2a | 1.4 | 0.8 |
| Traf1 | | 0.521 |
| Traf2 | | 0.16 |
| Trim47 | 0.183 | |
| Trp73 | | 0.741 |
| Tubb3 | | −0.195 |
| Txnrd1 | | 0.0889 |
| Tyrobp | 0.615 | 0.414 |
| Vav1 | 0.378 | |
| Vim | 0.253 | 0.319 |

### Example 2. Exposure Response of sCSF1R to Ab-1 in a Primate Model

Ab-1, a TREM2 agonist targeting microglia in the brain, was administered either intravenously (IV) or intrathecally (IT) in a single dose to Cynomolgus monkeys. Free Ab-1, chemokine, and soluble Colony-Stimulating Factor 1 Receptor (sCSF1R) analysis in CSF samples was conducted. Upon single intravenous administration at 2, 20, and 200 mg/kg, and single intrathecal catheter administration at 0.2 or 2 mg/animal, Ab-1 was well tolerated and was found to penetrate the CSF to a reasonable degree.

### Ab-1 Preparation Details

Dosing formulations were prepared at appropriate concentrations to meet dose level requirements. The pH was measured and adjusted as needed to 5.2±0.2 using HCl. The test article diluent, 15 mM Sodium acetate, 9% (w/v) sucrose, 0.01% (w/v) polysorbate 80, was prepared aseptically on the day prior to dosing and filtered using a 0.22 pM polyvinylidene fluoride (PVDF) filter. The Ab-1 test article was thawed overnight under refrigerated conditions prior to dosing. The dose formulations were prepared on the day of dosing by mixing the test article with the diluent and filtered through a 0.22 pM PVDF filter. A 12-hour expiration was set from the time of formulation completion and stored refrigerated (2° C. to 8° C.) until dosing. The test material was allowed to equilibrate to room temperature for at least 30 minutes prior to dose administration.

TABLE 7
Analysis of Ab-1 in Dosing Formulations-Day 1

| | | Nominal | Average Calculated |
|---|---|---|---|
| | Dose Level | Concentration | Concentration |
| Group | (mg/kg) | (mg/mL) | (mg/mL) |
| 1 | 2 | 1 | 0.9746 |
| 2 | 20 | 8 | 7.9462 |
| 3 | 200 | 80 | 77.5497 |
| 4 | 0.2 | 0.134 | 0.1148 |
| 5 | 2 | 1.34 | 1.2453 |

### Animal Handling

During testing, Cynomolgus monkeys were individually housed during designated study procedures/activities. Psychological/environmental enrichment was provided according to standard procedures. Target temperatures of 64° F. to 84° F. with a target relative humidity of 30% to 70% were maintained. A 12-hour light/12-hour dark cycle was maintained, except when interrupted for designated procedures. Lab Diet™ (Certified Primate Diet #5048, PMI Nutrition International, Inc.) was available to the monkeys twice a day except during designated periods. Enrichment foods were provided on a regular basis. Tap water was available ad libitum to each animal via an automatic watering system. Veterinary care was available throughout the course of the study, and animals were examined by the veterinary staff as warranted by clinical signs or other changes. No animals died during the course of the study. Intrathecal catheters were surgically implanted using standard laminectomy procedures

### Experimental Design

The test article was administered once on Day 1 via IV injection (Groups 1 to 3) or IT administration (Groups 4 and 5). The dose levels were 2, 20 and 200 mg/kg via IV injection at a dose volume of 2.0 and 2.5 mL/kg or 0.2 and 2 mg/animal via IT administration at a dose volume of 1.5 mL. Individual doses were based on the most recent body weights for IV administration. Each group consisted of four subjects, two male and two female. Table 8 outlines the overall experimental design and dosage levels.

TABLE 8
Experimental Design

| | | Dose | Dose | Dose Conc. | Dose Vol |
|---|---|---|---|---|---|
| Group | Test Material | Route | (mg/kg) | (mg/mL) | (mL/kg) |
| 1 | Low | IV | 2 | 1 | 2.0 |
| 2 | Mid | IV | 20 | 8 | 2.5 |
| 3 | High | IV | 200 | 80 | 2.5 |
| 4 | Match IV | IT | 0.2 | 0.134 | 1.5 |
| | High | | | | |
| 5 | Exceed IV | IT | 2 | 1.34 | 1.5 |
| | High | | | | |

### CSF Sample Collection

CSF samples (0.25 mL) were collected from all animals via the IT port for Chemokine and sCSF1R analyses. A target volume of CSF (0.25 mL) was collected at time points Day −2, 4 hrs, 12 hrs, 24 hrs, 32 hrs, 48 hrs, 72 hrs, 96 hrs and 168 hrs. The port was positive pressure locked with aCSF or sterile PBS post collection. CSF samples were collected and placed on wet ice. All aliquots were stored frozen at −60° C. to −90° C.

### Pharmacodynamic Results

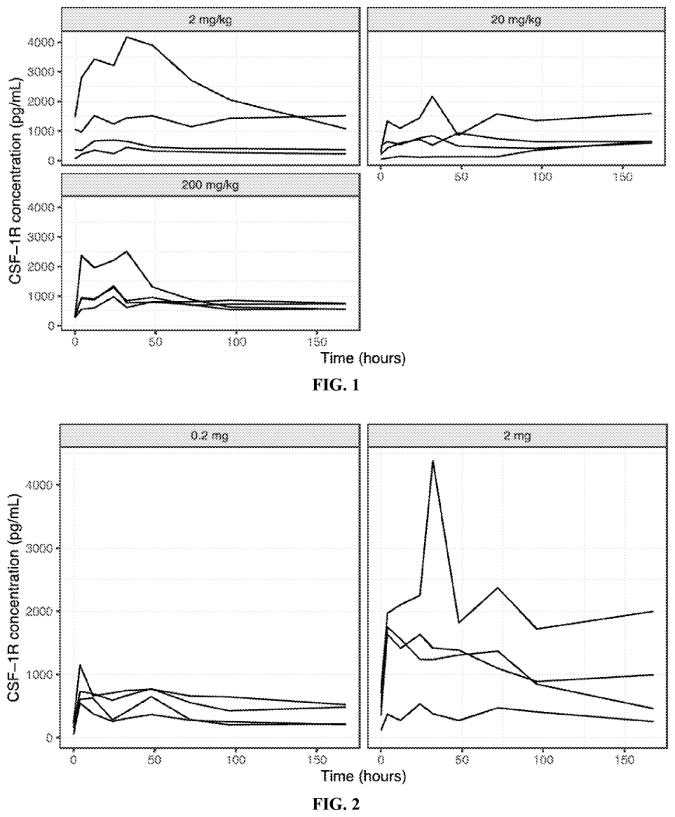

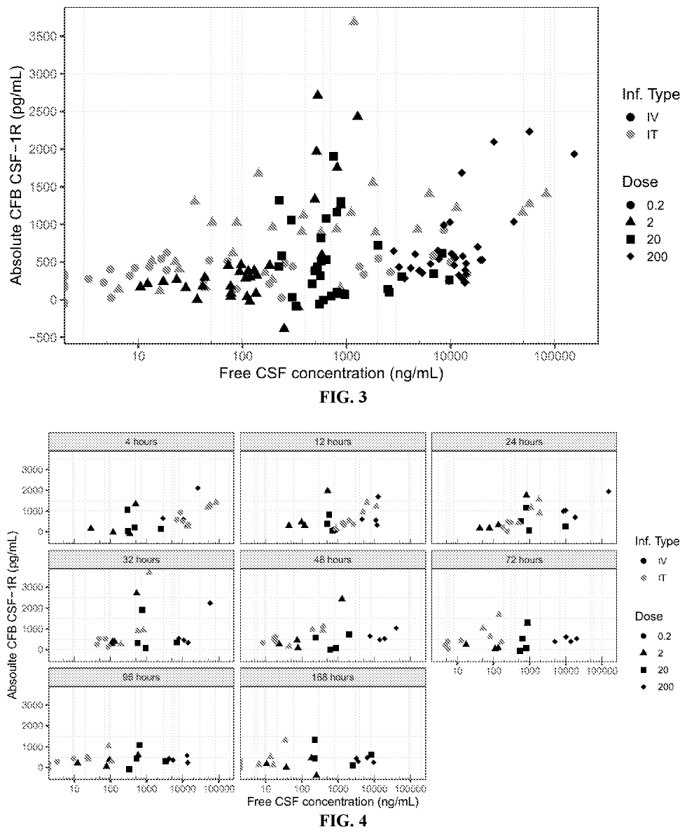

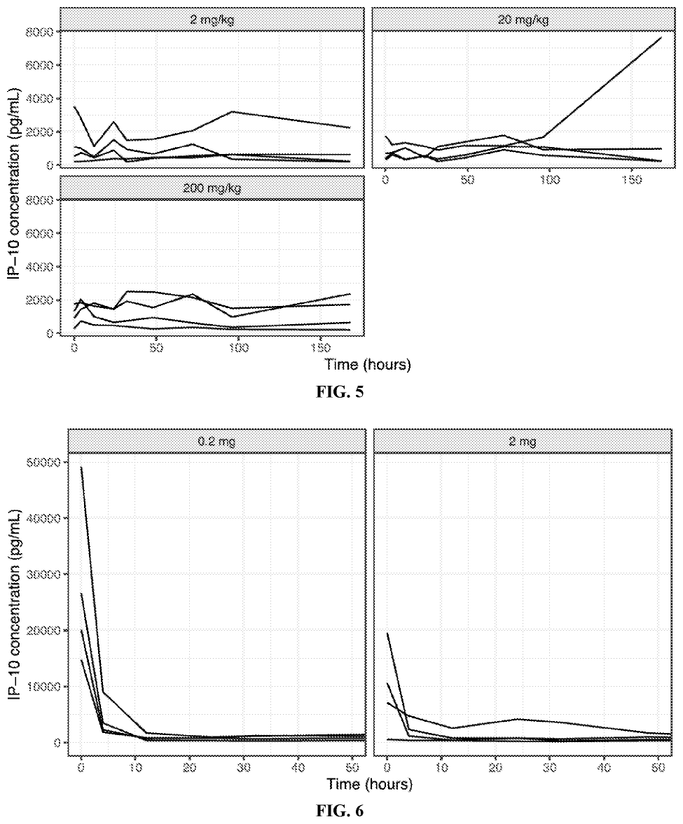

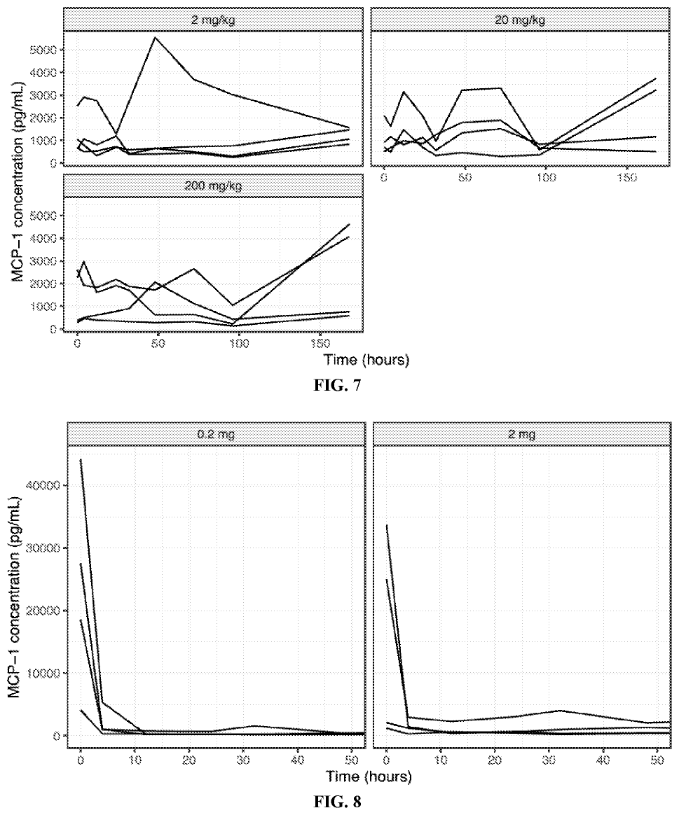

CSF samples were measured for free Ab-1, sCSF1R, IP-10, and MCP-1 concentrations as a function of time. sCSF1R, IP-10, and MCP-1 concentrations were determined using a Simoa-based immunoassay protocol. The concentration of sCSF1R, IP-10, and MCP-1 were plotted versus time. The change-from-baseline (CFB) concentration of sCSF1R was also plotted against free Ab-1 concentration. The concentration-time profiles for sCSF1R after IV administration and IT administration are shown in FIGS. 1 and 2, respectively. The change-from-baseline (CFB) concentration of sCSF1R plotted vs. the concentration of Ab-1 is shown in FIG. 3, as a combination of all data points, and in FIG. 4, where the data is broken up by time point, post-dose. FIG. 4 shows that the exposure-response is most pronounced at earlier timepoints (<24 hours) with a flat exposure response at later timepoints, suggestive of an indirect response effect of Ab-1 on sCSF1R. The concentration-time profiles for IP-10 after IV administration and IT administration are shown in FIGS. 5 and 6, respectively. The concentration-time profiles for MCP-1 after IV administration and IT administration are shown in FIGS. 7 and 8, respectively. No significant exposure-response was observed between IP-10 or MCP-1 and free Ab-1 and therefore this data was not included.

We claim:

1. A method of identifying a patient with a condition or disorder associated with microglial dysfunction who will benefit from treatment with a TREM2 agonist, comprising:
(a) obtaining a first biological sample from the patient prior to administration of the TREM2 agonist to the patient;
(b) measuring a level in the first biological sample of one or more biomarkers selected from those in Table A*;
(c) administering to the patient an effective amount of a TREM2 agonist;
(d) obtaining a second biological sample from the patient after administration of the TREM2 agonist to the patient;
(e) measuring a level in the second biological sample of one or more biomarkers selected from those in Table A*; and
(f) optionally, proceeding with further administration of the TREM2 agonist to the patient, if the change in the level of the one or more biomarkers in the first biological sample and the second biological sample indicates that the patient would benefit from treatment with the TREM2 agonist.

2. A method of predicting a treatment response to a TREM2 agonist for a condition or disorder associated with microglial dysfunction in a patient, comprising the steps of:
(a) obtaining a first biological sample from the patient prior to administration of the TREM2 agonist to the patient;
(b) measuring a level in the first biological sample of one or more biomarkers selected from those in Table A*;
(c) treating the biological sample from the patient or a reference sample;
(d) measuring a level in the treated biological sample of one or more biomarkers selected from those in Table A*;
(e) comparing one of more biomarkers in the pre-treatment biological sample with one or more biomarkers in the treated biological sample or treated reference sample; and
(f) optionally, proceeding with administration of the TREM2 agonist to the patient, if such administration is predicted to have an equivalent or higher likelihood of success relative to an alternative method of treating the condition or disorder;
wherein the biomarker change in response to step (c) is predictive of the likelihood of successful treatment of the condition or disorder based on a greater or lesser biomarker change compared with one or more similar patients and as evaluated using one or more of the biomarkers.

3. A method of treating a condition or disorder associated with microglial dysfunction with a TREM2 agonist, comprising:
(a) obtaining a first biological sample from the patient prior to administration of the TREM2 agonist to the patient;
(b) measuring a level in the first biological sample of one or more biomarkers selected from those in Table A*;
(c) administering to the patient an effective amount of a TREM2 agonist;
(d) obtaining a second biological sample after administration of the TREM2 agonist to the patient; and
(e) measuring a level in the second biological sample of one or more biomarkers selected from those in Table A*;
wherein when the level of one or more biomarkers selected from those in Table A* is higher in the second biological sample from the patient than in the first biological sample from the patient, then the patient is administered one or more additional doses of the TREM2 agonist.

4. The method of claim 3, wherein the one or more biomarkers are selected from those in Table C*.

5. A method of treating a condition or disorder associated with microglial dysfunction with a TREM2 agonist, comprising:
(a) obtaining a first biological sample from the patient prior to administration of the TREM2 agonist to the patient;
(b) measuring a level in the first biological sample of one or more biomarkers selected from those in Table A*;
(c) administering to the patient an effective amount of a TREM2 agonist;
(d) obtaining a second biological sample after administration of the TREM2 agonist to the patient; and
(e) measuring a level in the second biological sample of one or more biomarkers selected from those in Table A*;
wherein when the level of one or more biomarkers selected from those in Table A* is lower in the second biological sample from the patient than in the first biological sample from the patient, then the patient is administered one or more additional doses of the TREM2 agonist.

6. The method of claim 5, wherein the one or more biomarkers are selected from those in Table D*.

7. A method of monitoring a patient response to a TREM2 agonist, comprising the steps of:
(a) obtaining a first biological sample from the patient prior to administration of the TREM2 agonist to the patient;
(b) measuring a level in the first biological sample from the patient of one or more biomarkers selected from those in Table A*;
(c) administering to the patient an effective amount of a TREM2 agonist;
(d) obtaining a one or more subsequent biological samples from the patient after administration of the TREM2 agonist to the patient; and
(e) measuring a level in the subsequent biological sample(s) of one or more biomarkers selected from those in Table A*;
wherein the levels of one of more biomarkers in the first biological sample and subsequent biological samples can be compared and changes in one or more of the biomarkers indicate a patient response.

8. The method of any one of claims 1-7, wherein the one or more biomarkers comprise NFL.

9. The method of any one of claims 1-8, wherein the one or more biomarkers comprise sTREM2.

10. The method of any one of claims 1-9, wherein the one or more biomarkers comprise sCSF1R.

11. The method of any one of claims 1-10, wherein the one or more biomarkers comprise SPP1.

12. The method of any one of claims 1-11 wherein the one or more biomarkers comprise IL-IRA.

13. The method of any one of claims 1-12, wherein the one or more biomarkers comprise CSF2.

14. The method of any one of claims 1-13, wherein the one or more biomarkers comprise Chitotriosidate.

15. The method of any one of claims 1-14, wherein the one or more biomarkers comprise IP10.

16. The method of any one of claims 1-7, wherein the one or more biomarkers are selected from those in Table B.

17. The method of any one of claims 1-16, wherein the condition or disorder associated with microglial dysfunction is a condition or disorder associated with TREM2 deficiency or loss of TREM2 function.

18. The method of claim 17, wherein the condition or disorder associated with TREM2 deficiency or loss of TREM2 function is Nasu-Hakola disease, Alzheimer's disease, frontotemporal dementia, multiple sclerosis, Guillain-Barre syndrome, amyotrophic lateral sclerosis (ALS), Parkinson's disease, traumatic brain injury, spinal cord injury, systemic lupus erythematosus, rheumatoid arthritis, prion disease, stroke, osteoporosis, osteopetrosis, osteosclerosis, skeletal dysplasia, dysosteoplasia, Pyle disease, cerebral autosomal dominant arteriopathy with subcortical infarcts and leukoencephalopathy, cerebral autosomal recessive arteriopathy with subcortical infarcts and leukoencephalopathy, cerebroretinal vasculopathy, or metachromatic leukodystrophy.

19. The method of any one of claims 1-16, wherein the condition or disorder associated with microglial dysfunction is a condition or disorder associated with dysfunction of colony stimulating factor 1 receptor (CSF1R).

20. The method of claim 19, wherein the condition or disorder associated with dysfunction of CSF1R is axonal spheroids and pigmented glia (ALSP), hereditary diffuse leukoencephalopathy with axonal spheroids (HDLS), pigmentary orthochromatic leukodystrophy (POLD), pediatric-onset leukoencephalopathy, congenital absence of microglia, or brain abnormalities neurodegeneration and dysosteosclerosis (BANDDOS).

21. The method of any one of claims 1-16, wherein the condition or disorder associated with microglial dysfunction is a condition or disorder associated with dysfunction of ATP-binding cassette transporter 1 (ABCD1).

22. The method of claim 21, wherein the condition or disorder associated with dysfunction of ABCD1 is X-linked adrenoleukodystrophy (x-ALD), childhood cerebral adrenoleukodystrophy (cALD), Globoid cell leukodystrophy (also known as Krabbe disease), Metachromatic leukodystrophy (MLD), Cerebral autosomal dominant arteriopathy with subcortical infarcts and leukoencephalopathy (CADASIL), Vanishing white matter disease (VWM), Alexander disease, fragile X-associated tremor ataxia syndrome (FXTAS), adult-onset autosomal dominant leukodystrophy (ADLD), and X-linked Charcot-Marie-Tooth disease (CMTX).

23. The method of any one of claims 1-22, wherein the TREM2 agonist is an anti-hTREM2 antibody.

24. The method of claim 23, wherein the anti-hTREM2 antibody comprises a light chain variable region comprising a CDRL1 having an amino acid sequence according to SEQ ID NO:6; a CDRL2 having an amino acid sequence according to SEQ ID NO:7; and a CDRL3 having an amino acid sequence according to SEQ ID NO:8, and a heavy chain variable region comprising a CDRH1 having an amino acid sequence according to SEQ ID NO: 10; a CDRH2 having an amino acid sequence according to SEQ ID NO: 11; and a CDRH3 having an amino acid sequence according to SEQ ID NO: 12.

25. The method of claim 23, wherein the anti-hTREM2 antibody comprises a light chain variable region having an amino acid sequence according to SEQ ID NO: 5, and a heavy chain variable region having an amino acid sequence according to SEQ ID NO: 9.

26. The method of claim 24 or 25, wherein the anti-hTREM2 antibody is an IgG, optionally an IgG₁.

27. The method of any one of claims 24-26, wherein the anti-hTREM2 antibody comprises a kappa light constant region.

28. The method of any one of claims 24-27, wherein the anti-hTREM2 antibody is an IgG₁ comprising a variant constant region having one or more mutations selected from R292C, N297G, V302C, D356E, or L358M, according to EU numbering.

29. The method of any one of claims 24-28, wherein the anti-hTREM2 antibody comprises a light chain having an amino acid sequence according to SEQ ID NO: 13, and a heavy chain variable region having an amino acid sequence according to SEQ ID NO: 16.

30. The method of any one of claims 1-22, wherein the TREM2 agonist is a small molecule TREM2 agonist.


SEQUENCE LISTING (20 sequences)

SEQ ID NO: 1 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 2 - protein, 173 aa, Homo sapiens
MEPLRLLILLFVTELSGAHNTTVFQGVAGQSLQVSCPYDSMKHWGRRKAWCRQLGEKGPCQRWSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTS

SEQ ID NO: 3 - protein, 112 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYY

SEQ ID NO: 4 - protein, 234 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFNIVILLAGGFLSKSLVFSVLFAVTLRSFVP

SEQ ID NO: 5 - protein, 107 aa, Artificial Sequence
  Synthetic polypeptide
EIVMTQSPATLSVSPGERATLSCRASQSVSSNLAWFQQKPGQAPRLLIYGASTRATGIPARFSGSGSGTEFTLTISSLQPEDFAVYYCLQDNNFPPTFGQGTKVDIK

SEQ ID NO: 6 - protein, 11 aa, Artificial Sequence
  Synthetic peptide
RASQSVSSNLA

SEQ ID NO: 7 - protein, 7 aa, Artificial Sequence
  Synthetic peptide
GASTRAT

SEQ ID NO: 8 - protein, 9 aa, Artificial Sequence
  Synthetic peptide
LQDNNFPPT

SEQ ID NO: 9 - protein, 121 aa, Artificial Sequence
  Synthetic polypeptide
EVQLVQSGAEVKKPGESLKISCKGSGYSFTSYWIGWVRQMPGKGLEWMGIIYPGDADARYSPSFQGQVTISADKSISTAYLQWSSLKASDTAMYFCARRRQGIFGDALDFWGQGTLVTVSS

SEQ ID NO: 10 - protein, 5 aa, Artificial Sequence
  Synthetic peptide
SYWIG

SEQ ID NO: 11 - protein, 17 aa, Artificial Sequence
  Synthetic polypeptide
IIYPGDADARYSPSFQG

SEQ ID NO: 12 - protein, 12 aa, Artificial Sequence
  Synthetic polypeptide
RRQGIFGDALDF

SEQ ID NO: 13 - protein, 214 aa, Artificial Sequence
  Synthetic polypeptide
EIVMTQSPATLSVSPGERATLSCRASQSVSSNLAWFQQKPGQAPRLLIYGASTRATGIPARFSGSGSGTEFTLTISSLQPEDFAVYYCLQDNNFPPTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 236 aa, Artificial Sequence
  Synthetic polypeptide
MDMRVPAQLLGLLLLWLRGARCEIVMTQSPATLSVSPGERATLSCRASQSVSSNLAWFQQKPGQAPRLLIYGASTRATGIPARFSGSGSGTEFTLTISSLQPEDFAVYYCLQDNNFPPTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 214 aa, Artificial Sequence
  Synthetic polypeptide
EIVMTQSPATLSVSPGERATLSCRASQSVSSNLAWFQQKPGQAPRLLIYGASTRATGIPARFSGSGSGTEFTLTISSLQPEDFAVYYCLQDNNFPPTFGQGTKVDIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 16 - protein, 451 aa, Artificial Sequence
  Synthetic polypeptide
EVQLVQSGAEVKKPGESLKISCKGSGYSFTSYWIGWVRQMPGKGLEWMGIIYPGDADARYSPSFQGQVTISADKSISTAYLQWSSLKASDTAMYFCARRRQGIFGDALDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 473 aa, Artificial Sequence
  Synthetic polypeptide
MDMRVPAQLLGLLLLWLRGARCEVQLVQSGAEVKKPGESLKISCKGSGYSFTSYWIGWVRQMPGKGLEWMGIIYPGDADARYSPSFQGQVTISADKSISTAYLQWSSLKASDTAMYFCARRRQGIFGDALDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 445 aa, Artificial Sequence
  Synthetic polypeptide
EVQLVQSGAEVKKPGESLKISCKGSGYSFTSYWIGWVRQMPGKGLEWMGIIYPGDADARYSPSFQGQVTISADKSISTAYLQWSSLKASDTAMYFCARRRQGIFGDALDFWGQGTLVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 19 - protein, 445 aa, Artificial Sequence
  Synthetic polypeptide
EVQLVQSGAEVKKPGESLKISCKGSGYSFTSYWIGWVRQMPGKGLEWMGIIYPGDADARYSPSFQGQVTISADKSISTAYLQWSSLKASDTAMYFCARRRQGIFGDALDFWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFGSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 20 - protein, 214 aa, Artificial Sequence; identical to SEQ ID NO: 15
  Synthetic polypeptide